SEQUENCE LISTING (40 sequences)

SEQ ID NO: 1 - DNA, 1000 bp, synthetic construct
  source 1..1000: mol_type = other DNA
ttagtaggaactccccgagagaatgtggtggcacgatactacggtgcgattatactgcaaaaagcctgcaactcgcgagaaatgtattaagccaaaccaattttgatctaagttggacatgcagtgtgcgtccgatgtcagtttgggctctccctttaggcgcaggcgggtacagacgttcggttttcattgacatctgccattcaattcgagcagtaaacacataacttaacaattcttattcaggtactctggcgatattttcaagcgagtggagaatgccctcaaacaagatgaactgaatgcacagccgacgttcccacttatctgcctttttcactacatgggaacgacatcgacacaaaaaaaacggttagcatagaagttatccacactgtagaccacccattaaggcgtcagtgaatggataccttctgtttggtacgccgacacaatcagtggtgtatctatcctgtttggcagacggcaaaatatggagtggataaaaactagaactgatatgagatggggtggcgcctttccgagagggttcggatgcttgtaaacgtatcagattgtgagccgataaaacaaaatgacggtacgcaattggtctctcccctttacccgaacatgtgtggactgcttaaaataaaaaaaactcacgatgtggcgcatatactgagggtcgaatccatgatacactgcttcatttgccgatgactttatggcggcatatatggatgtcactatgaagaccagaaaatacttctgcttgcgatccgggagaacgagtagctgattcactctgtccgctcggggataagagggccagatcctaggctcgcatgtgacgtgcacctaattatacatttgctcccgttccgctaaacgtcttaatccttaccgtaaggtgtcgttagcaagcgcattctgacttcggaccatactaaccagtagagtcgtaatagcaccataggattaacgctgtcctggtgtt

SEQ ID NO: 2 - DNA, 1000 bp, synthetic construct
  source 1..1000: mol_type = other DNA
ggtgcgactctgctcgcactctaacagtccatacccttggtcggaggcgggcctacatgacagatacgtaattgacgctcttcttgacctccggatccctgatcattcaatgaggactgtgaacgcagtaatgaacattagtcgccaggagttagagcggcctcacgatcttgaatggagtaggttctttgcggtcccctcgcgacgctatctacttgacgtgtctgaggtcatatattgagtcgtaagcaggtgagacgccaacgcctatacaagagacgggacaattacgagtaatggctgtgctgcattttgcgagttccttcgcagggaattgtacgttagataggagaggcgagctcgtacaacaagcgttcgtgcacgcgttaatccacccacggcgggtcagccaatcccgtgctatacgcagactcttggatgtagcaggtcgtcgagcagttagccaagtgtgagagctggtagctctcctcgtaggggctacaaccaagtgctggcccgccactagttcctgcactcagtcgacacagtgaatgtttcgccgtagcgcatcaagtacgctctagtccgaaagaatgcgctcaaagtaaggccctccttctgcaaaagttatcaaccagggagcagtaaaaccatgacgtcggtgacatggaggtcttccagcgtagctgcctccgtagaataaggtctacgacatgataaatagcctacacctaatctggcggaatccgtaaactctccagttactcccgatcgtttagacccacattgcgccacaaggtggataaggataagcagtgtcttgcgtccactgaggggctggcattttccgcctgactagttttctatatgacaactgtggtattcgacgaagctcactaaatcaacgagatatcaggggaccggggcgtaatattatgacgaaataggcgcggataatataagcacagtaccctatgcacgggaaatcatcagcgaatga

SEQ ID NO: 3 - DNA, 25 bp, synthetic construct
  source 1..25: mol_type = other DNA
tttcactgagcgacatacactggca

SEQ ID NO: 4 - DNA, 25 bp, synthetic construct
  source 1..25: mol_type = other DNA
tttctgaaacctggaccaacattac

SEQ ID NO: 5 - DNA, 25 bp, synthetic construct
  source 1..25: mol_type = other DNA
tttcaacattgttctgactgggtcc

SEQ ID NO: 6 - DNA, 25 bp, synthetic construct
  source 1..25: mol_type = other DNA
tttctttcgaagatcgttactcgtg

SEQ ID NO: 7 - DNA, 25 bp, synthetic construct
  source 1..25: mol_type = other DNA
tttccagctcgctccggccgagtat

SEQ ID NO: 8 - DNA, 25 bp, synthetic construct
  source 1..25: mol_type = other DNA
tttccttggagagcgatatgtctca

SEQ ID NO: 9 - DNA, 25 bp, synthetic construct
  source 1..25: mol_type = other DNA
tttcacgcggtaccgggttgcaaac

SEQ ID NO: 10 - DNA, 25 bp, synthetic construct
  source 1..25: mol_type = other DNA
tttccacgttgactaactaaccggg

SEQ ID NO: 11 - DNA, 25 bp, synthetic construct
  source 1..25: mol_type = other DNA
tttcactagaacagtagatggcttg

SEQ ID NO: 12 - DNA, 25 bp, synthetic construct
  source 1..25: mol_type = other DNA
tttcatagatagacgaagagcgtaa

SEQ ID NO: 13 - DNA, 25 bp, synthetic construct
  source 1..25: mol_type = other DNA
tttcgaagaagcggtttaagggtct

SEQ ID NO: 14 - DNA, 125 bp, synthetic construct
  source 1..125: mol_type = other DNA
tttcactgagcgacatacactggcatttctgaaacctggaccaacattactttcatagatagacgaagagcgtaatttcacgcggtaccgggttgcaaactttcaacattgttctgactgggtcc

SEQ ID NO: 15 - DNA, 125 bp, synthetic construct
  source 1..125: mol_type = other DNA
tttcactgagcgacatacactggcatttctgaaacctggaccaacattactttcatagatagacgaagagcgtaatttcacgcggtaccgggttgcaaactttctttcgaagatcgttactcgtg

SEQ ID NO: 16 - DNA, 125 bp, synthetic construct
  source 1..125: mol_type = other DNA
tttcactgagcgacatacactggcatttctgaaacctggaccaacattactttcgaagaagcggtttaagggtcttttccacgttgactaactaaccgggtttccagctcgctccggccgagtat

SEQ ID NO: 17 - DNA, 125 bp, synthetic construct
  source 1..125: mol_type = other DNA
tttcactgagcgacatacactggcatttctgaaacctggaccaacattactttcgaagaagcggtttaagggtcttttcactagaacagtagatggcttgtttccttggagagcgatatgtctca

SEQ ID NO: 18 - RNA, 42 nt, synthetic construct
  source 1..42: mol_type = other RNA
  misc_RNA 1..21: note = tracrRNA
  misc_RNA 22..42: note = crRNA
ggaatttctactcttgtagatactgagcgacatacactggca

SEQ ID NO: 19 - RNA, 42 nt, synthetic construct
  source 1..42: mol_type = other RNA
  misc_RNA 1..21: note = tracrRNA
  misc_RNA 22..42: note = crRNA
ggaatttctactcttgtagatacgcggtaccgggttgcaaac

SEQ ID NO: 20 - RNA, 42 nt, synthetic construct; identical to SEQ ID NO: 19
  source 1..42: mol_type = other RNA
  misc_RNA 1..21: note = tracrRNA
  misc_RNA 22..42: note = crRNA

SEQ ID NO: 21 - RNA, 42 nt, synthetic construct
  source 1..42: mol_type = other RNA
  misc_RNA 1..21: note = tracrRNA
  misc_RNA 22..42: note = crRNA
ggaatttctactcttgtagatcttggagagcgatatgtctca

SEQ ID NO: 22 - DNA, 155 bp, synthetic construct
  source 1..155: mol_type = other DNA
acgctgtcctggtgtttttcactgagcgacatacactggcatttctgaaacctggaccaacattactttcatagatagacgaagagcgtaatttcacgcggtaccgggttgcaaactttcaacattgttctgactgggtccggtgcgactctgct

SEQ ID NO: 23 - DNA, 155 bp, synthetic construct
  source 1..155: mol_type = other DNA
tgcgacaggaccacaaaaagtgactcgctgtatgtgaccgtaaagactttggacctggttgtaatgaaagtatctatctgcttctcgcattaaagtgcgccatggcccaacgtttgaaagttgtaacaagactgacccaggccacgctgagacga

SEQ ID NO: 24 - DNA, 155 bp, synthetic construct
  source 1..155: mol_type = other DNA
acgctgtcctggtgtttttcactgagcgacatacactggcatttctgaaacctggaccaacattactttcatagatagacgaagagcgtaatttcacgcggtaccgggttgcaaactttctttcgaagatcgttactcgtgggtgcgactctgct

SEQ ID NO: 25 - DNA, 155 bp, synthetic construct
  source 1..155: mol_type = other DNA
tgcgacaggaccacaaaaagtgactcgctgtatgtgaccgtaaagactttggacctggttgtaatgaaagtatctatctgcttctcgcattaaagtgcgccatggcccaacgtttgaaagaaagcttctagcaatgagcacccacgctgagacga

SEQ ID NO: 26 - DNA, 155 bp, synthetic construct
  source 1..155: mol_type = other DNA
acgctgtcctggtgttatactcggccggagcgagctggaaacccggttagttagtcaacgtggaaaagacccttaaaccgcttcttcgaaagtaatgttggtccaggtttcagaaatgccagtgtatgtcgctcagtgaaaggtgcgactctgct

SEQ ID NO: 27 - DNA, 155 bp, synthetic construct
  source 1..155: mol_type = other DNA
tgcgacaggaccacaatatgagccggcctcgctcgacctttgggccaatcaatcagttgcaccttttctgggaatttggcgaagaagctttcattacaaccaggtccaaagtctttacggtcacatacagcgagtcactttccacgctgagacga

SEQ ID NO: 28 - DNA, 155 bp, synthetic construct
  source 1..155: mol_type = other DNA
acgctgtcctggtgtttgagacatatcgctctccaaggaaacaagccatctactgttctagtgaaaagacccttaaaccgcttcttcgaaagtaatgttggtccaggtttcagaaatgccagtgtatgtcgctcagtgaaaggtgcgactctgct

SEQ ID NO: 29 - DNA, 155 bp, synthetic construct
  source 1..155: mol_type = other DNA
tgcgacaggaccacaaactctgtatagcgagaggttcctttgttcggtagatgacaagatcacttttctgggaatttggcgaagaagctttcattacaaccaggtccaaagtctttacggtcacatacagcgagtcactttccacgctgagacga

SEQ ID NO: 30 - DNA, 42 bp, synthetic construct
  source 1..42: mol_type = other DNA
ggaatttctactcttgtagatcgacagcttgaccgtgctcac

SEQ ID NO: 31 - DNA, 42 bp, synthetic construct
  source 1..42: mol_type = other DNA
ggaatttctactcttgtagattgaaaggtacccctgtgacca

SEQ ID NO: 32 - DNA, 42 bp, synthetic construct
  source 1..42: mol_type = other DNA
ggaatttctactcttgtagatgattgtcgcgggaccacaagt

SEQ ID NO: 33 - DNA, 125 bp, synthetic construct
  source 1..125: mol_type = other DNA
tttcactgagcgacatacactggcatttctgaaacctggaccaacattactttcatagatagacgaagagcgtaatttctgaaaggtacccctgtgaccatttcgattgtcgcgggaccacaagt

SEQ ID NO: 34 - DNA, 125 bp, synthetic construct
  source 1..125: mol_type = other DNA
tttcactgagcgacatacactggcatttctgaaacctggaccaacattactttcgaagaagcggtttaagggtcttttcactagaacagtagatggcttgtttccgacagcttgaccgtgctcac

SEQ ID NO: 35 - DNA, 52 bp, synthetic construct
  source 1..52: mol_type = other DNA
ccataggattaacgctgtcctggtgttttccgttaaattgccgtcgtcagcc

SEQ ID NO: 36 - DNA, 36 bp, synthetic construct
  source 1..36: mol_type = other DNA
gcaatttaacggaaaacaccaggacagcgttaatcc

SEQ ID NO: 37 - DNA, 52 bp, synthetic construct; identical to SEQ ID NO: 35
  source 1..52: mol_type = other DNA

SEQ ID NO: 38 - DNA, 33 bp, synthetic construct
  source 1..33: mol_type = other DNA
agagtcgcaccgagagagttgaacctggacgcc

SEQ ID NO: 39 - DNA, 42 bp, synthetic construct
  source 1..42: mol_type = other DNA
gcgcggcgtccaggttcaactctctcggtgcgactctgctcg

SEQ ID NO: 40 - DNA, 20 bp, synthetic construct
  source 1..20: mol_type = other DNA
acggattccgccagattagg

(19) United States
(12) Patent Application Publication
(10) Pub. No.: US 2026/0297626 A1
(43) Pub. Date: Oct. 1, 2026

(54) GENERATION OF MULTI-COPY HOST CELLS

(71) Applicant: Novozymes A/S, Bagsvaerd (DK)

(72) Inventor: Carsten Lillelund OLSEN, Bagsvaerd (DK)

(73) Assignee: Novozymes A/S, Bagsvaerd (DK)

(21) Appl. No.: 19/477,316

(22) PCT Filed: Apr. 18, 2024
(86) PCT No.: PCT/EP2024/060591

§ 371 Date: Oct. 21, 2025

(30) Foreign Application Priority Data
Apr. 21, 2023 (EP) 23169124.7

(51) Int. Cl. C12N 15/90; C12N 9/18; C12N 9/22; C12N 15/11

(52) U.S. Cl. CPC C12N 15/902; C12N 9/18; C12N 9/22; C12N 15/11; C12N 2310/20

(57) ABSTRACT

The present invention relates to host cells comprising in its genome at least two landing pad polynucleotides (LPP), methods of generating multi-copy host cells, and methods of producing a polypeptide of interest with said multi-copy host cells.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to host cells comprising in its genome at least two landing pad polynucleotides (LPP), methods of generating multi-copy host cells, and methods of producing a polypeptide of interest with said host cells.

### Description of the Related Art

Recombinant host cells are widely used for recombinant protein production. For industrial and commercial purposes, the productivity of the applied cell systems, i.e., the production of total protein per fermentation unit, is an important factor of production costs. Traditionally, yield increases have been achieved through mutagenesis and screening for increased production of proteins of interest. However, this approach is mainly only useful for the overproduction of endogenous proteins in isolates containing the enzymes of interest. Therefore, for each new protein or enzyme product, a lengthy strain and process development program is required to achieve improved productivities.

To obtain high-level expression of a particular gene, a well-established procedure is targeting multiple copies of the recombinant gene constructs to the locus of a highly expressed endogenous gene. However, generation of such multi-copy host cells is difficult to control (i.e., number and location of GOI copies, and genome stability) and often a lengthy process requiring numerous iterations of transfection/transformation and subsequent sequencing. Also, the efficiency of targeted integration is often low, making multi-copy strain generation laborious and expensive.

Multi-copy host cells can for example be generated using the flippase-based FLP-FRT system disclosed in WO16145084 (Novozymes A/S). However, known disadvantages of the FLP-FRT system are for example (i) the occurrence of unwanted recombination between FRT sites, (ii) the requirement of high DNA loads during transformation, and (iii) the requirement for marker genes in all of the landing pads.

Thus, it is of continuous interest to improve the generation of multi-copy host cells. The object of the present invention is to provide a modified host cell and a method for generating multi-copy host cells.

### SUMMARY OF THE INVENTION

The present invention is based on the surprising and inventive finding that host cells comprising multiple landing pads, each landing pad comprising homology arms and multiple protospacer sequences, can be efficiently used for targeted integration of multiple GOI copies. Said host cells can be used to generate multi-copy host cells via the method of the invention: synthetic protospacer array defined entry (SPADE). With the presence of multiple landing pads and multiple protospacer sequences, the SPADE system provides a versatile platform to generate host cells with desired copy numbers for gene(s) of interest to be integrated into the host cell genome.

Surprisingly, although not using any marker genes in the SPADE cells, high multi-copy integration efficiencies of 75-80% were observed (Examples 2-3). Most surprisingly, high multi-copy integration efficiency was achieved in only one round of transformation, i.e., in a one step fashion (Example 2, Examples 7-8, and Examples 10-11), resulting in shortened timelines for multi-copy host cell generation with up to 6 copies of the GOI. Importantly, the inventors have shown that homology arms as short as 100 bp are sufficient for the SPADE system (Example 9).

Furthermore, the inventors observed that with synthetic homology arms in the landing pads, potential silencing issues were eliminated, resulting in increased homologous recombination events.

A further surprising finding was that, by placing the landing pads in different orientation in the genome, i.e., some landing pads are present on the coding strand while other landing pads are present on the template strand (FIG. 3), unwanted recombination events between integration sites, which as described above is a major disadvantage in using the prior art FLP-FRT systems, can be significantly reduced. This design furthermore prevents unwanted landing pad/GOI deletions and/or chromosome arm swaps.

A further advantage of the SPADE system over prior art systems, e.g., the FLP-FRT system, is that the load of DNA can be reduced during transformation—which is an important advantage when toxic DNA/proteins are transformed. As shown in Example 4, using the SPADE technology GOI sequences previously integrated into landing pads can be “re-used” and integrated into “empty” landing pads to further increase the copy number of the host cell. The amount of DNA per transfection can be reduced to reduce DNA and/or protein toxicity for the host cell (and thereby maintaining desired host cell viabilities), whilst not compromising integration efficiency or GOI copy number.

Additionally, the SPADE system does not require a recombinase sequence present close (e.g., placed in cis) to the landing pads as is the case for the FLP/FRT system.

Also, the SPADE system allows to maintain some landing pads as “empty” landing pads, while other landing pads harbor the gene of interest. This further contributes to a versatile host cell generation platform and e.g., allows later integration of further (same or different) genes of interest into the genome if desired.

Advantageously, within a one or two integration steps the different landing pads can be used for integration of different GOIs, to obtain strains having varying ratios of two or more GOIs (see Example 8 and Example 12, respectively).

Lastly, the SPADE system allows to introduce linear PCR products comprising the different features of the system (e.g., GOI, 3′ homology arm, and 5′ homology arm), see e.g., Example 11. By doing so, no full plasmid constructs need to be cloned which results in a highly time-efficient strain construction.

In a first aspect, the present invention provides host cells comprising in their genome at least two landing pad polynucleotide (LPP) sequences, each LPP sequence comprising in 5′ to 3′ direction: a 5′ homology arm, two or more protospacer sequences, and a 3′ homology arm, wherein the polynucleotide sequence of the 5′ homology arm is identical for at least two LPP sequences, and wherein the polynucleotide sequence of the 3′ homology arm is identical for at least two LPP sequences.

The host cells of the first aspect have the advantage of being versatile starting points for generating multi-copy host cells, i.e. the cells can be used to integrated genes of interest into one, two or more, or into all LPPs of the host cell. The location and number of the LPPs in the host cell genome can be designed depending on the host cell type and the gene of interest which shall be integrated therein.

In a second aspect, the present invention provides a method for generating a multi-copy host cell, comprising the steps of
a) providing a host cell according to the first aspect,
b) delivering to the host cell a gene editing system comprising:
   a RNA-guided DNA nuclease,
   at least one guide RNA directed to one or more protospacer of the one or more LPP sequences,
a first donor polynucleotide comprising in 5′ to 3′ direction: a 5′ homology arm, a first polynucleotide encoding a first polypeptide of interest, and a 3′ homology arm, OR a set of first PCR fragments comprising a first 5′ homology arm, a first 3′ homology arm, and a first polynucleotide encoding a first polypeptide of interest, wherein the 3′ homology arm and/or first 3′ homology arm is identical to the 3′ homology arm of one or more LPP, and wherein the 5′ homology arm and/or first 5′ homology arm is identical to the 5′ homology arm of one or more LPP, and
c) cultivating the host cell at a first temperature allowing one or more first guide RNA of the at least one guide RNA to form a complex with the nuclease at one or more protospacer of one or more LPP, and wherein the complex promotes the homology directed repair by creating a double-strand break at or near the one or more protospacer sequence of the one or more LPP, wherein the homology directed repair comprises insertion of the first donor polynucleotide or set of first PCR fragments at the double-strand break.

Using the method of the second aspect, efficient and targeted integration of multiple gene copies into the host cell is enabled, either in a single step or in a multi-step approach.

In a third aspect, the present invention provides a method of producing a polypeptide of interest, comprising cultivating a multi-copy host cell under conditions conducive for production of the polypeptide, which multi-copy host cell is generated by the method according to the second aspect.

### BRIEF DESCRIPTION OF THE DRAWINGS

FIG. 1 shows a host cell of the invention comprising 4 landing pad polynucleotides (LPPs).

FIG. 2a shows the RNA-guided cutting of three LPPs in a host cell of the invention.

FIG. 2b shows homology-directed repair of the generated double strand breaks in a host cell of the invention.

FIG. 2c shows a host cell harboring three copies of a GOI in three LPPs.

FIG. 3 shows a detailed overview of the 4 LPPs including their PAM sequences and protospacers (homology arms are not fully represented), and the LPP either being present on the coding strand or on the template strand.

FIG. 4 shows an example of a set of PCR fragments used for transformation into a host cell with a SPADE LPP.

### DEFINITIONS

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Landing pad polynucleotide (LPP): The term “LPP” or landing pad polynucleotide, or landing pad, means a DNA sequence which comprises a 5′ homology arm, two or more protospacer sequences, and a 3′ homology arm. The LPP is inserted into the host cell genome on the desired chromosome and/or genome location. The LPP is then targeted with a nuclease and a gRNA at a protospacer of the LPP which results in a double-strand break at or near the targeted protospacer. Homology-directed repair takes place with a donor polynucleotide and/or PCR fragments. The donor polynucleotide or PCR fragments carry a gene of interest, and comprise homology arms being identical to the 5′ and 3′ homology arms of the targeted LPP. The repair results in integration of the donor polynucleotide and/or PCR fragments in the targeted LPP. The homology-directed repair is not limited to the presence of the donor polynucleotide and/or PCR fragments, but may instead/additionally take place with an already integrated gene of interest in a LPP having identical homology arms to the homology arms of the targeted LPP.

Homology arm: The term homology arm means a DNA sequence which is present at the 5′ arm (=5′ homology arm) and the 3′ arm (=3′ homology arm) of the LPP and the donor polynucleotide. Typically, the 3′ arm sequence is not identical to the 5′ arm sequence. The homology arms may have a length of at least 10 nt, at least 20 nt, at least 30 nt, at least 50 nt, at least 100 nt, 200 nucleotides (nt), at least 300 nt, at least 400 nt, at least 500 nt, at least 600 nt, at least 700 nt, at least 800 nt, at least 900 nt, or at least 1000 nt.

For the LPP, the 5′ arm is located upstream of the two or more protospacers, and the 3′ arm is located downstream of the two or more protospacers.

For the donor polynucleotide, the 5′ arm is located upstream of the first polynucleotide encoding the first polypeptide of interest, and the 3′ arm is located downstream of the first polynucleotide encoding the first polypeptide of interest.

Protospacer: The term protospacer or protospacer sequence means a DNA sequence located within the 5′ arm and the 3′ arm of an LPP. The protospacer is integrated into the host cell genome and is complementary to the spacer sequence of a guide RNA, which guide RNA can be used to create a double-strand cut at or near the protospacer sequence. The protospacer may have a length of between 14-36 nucleotides. DNA-guided nucleases may also require a PAM sequence present near the protospacer sequence. The PAM sequence (protospacer adjacent motif) is located directly downstream of the target sequence in the genomic DNA, on the non-target strand, i.e. on the strand opposite to the strand which comprises the protospacer sequence.

First protospacer: The term first protospacer means a protospacer which is present in all LPP of the host cell.

Second protospacer: The term second protospacer means a protospacer which is unique for only one LPP of the at least two LPPs in the host cell genome.

Third protospacer: The term third protospacer means a protospacer which is present in a third subset of the at least two LPPs.

Third subset of the at least two LPPs: The third subset of LPPs, means a subset of LPPs comprising a third protospacer which is present in two or more LPPs, and/or is present in all LPPs of the host cell.

Fourth protospacer: The term fourth protospacer means a protospacer which is present in a fourth subset of the at least two LPPs.

Fourth subset of the at least two LPPs: The fourth subset of LPPs, means a subset of LPPs comprising a fourth protospacer which is present in two or more LPPs, and/or is present in all LPPs of the host cell. Typically, the fourth subset consists of a number of LPPs which number of LPPs is different from the number of LPPs present in the third subset.

Fifth protospacer: The term fifth protospacer means a protospacer which is present in a fifth subset of the at least two LPPs.

Fifth subset of the at least two LPPs: The fifth subset of LPPs, means a subset of LPPs comprising a fifth protospacer which is present in two or more LPPs, and/or is present in all LPPs of the host cell. Typically, the fifth subset consists of a number of LPPs which number of LPPs is different from the number of LPPs present in the third subset, and also different from the number of LPPs present in the fourth subset.

RNA-guided DNA nuclease: RNA-guided DNA nucleases provide sequence-specific DNA cutting through base-pairing interactions between a guide RNA and target DNA (protospacer sequence) of the two or more LPPs. These nucleases include, but are not limited to Cas9, MAD7, and Cpf1. RNA-guided nucleases use base pairing to recognize and cleave target DNA with complementarity to the guide RNA. guide RNA and nuclease may be delivered to the host cell by transformation/transfection, either on the same plasmid or linearized DNA vector, or on different separate plasmids or DNA vectors. Additionally or alternatively, the nuclease may be delivered to the cell as protein, e.g. by protein transfection.

guide RNA: The guide RNA functions as a guide for the RNA-guided DNA nuclease. The guide RNA forms a complex with the nuclease and the target strand, to which the guide RNA is complementary. The guide RNA may comprise a spacer, which is complementary to the protospacer sequence of the DNA target, a short CRISPR RNA (crRNA), and a tracrRNA.

First guide RNA: The first guide RNA is a guide RNA that forms an editing complex most efficiently at the first temperature, and forms an editing complex less efficiently at other temperatures. Also, the first guide RNA comprises a spacer sequence which may be complementary to any of the first, second, third, fourth or fifth protospacers. The first guide RNA may be a 1st first guide RNA, a 2nd first guide RNA, 3rd first guide RNA, 4^{rd} first guide RNA, and/or a 5^{th} first guide RNA.

First donor polynucleotide: The first donor polynucleotide comprises a first polynucleotide which encodes a first polypeptide of interest. The first donor polynucleotide furthermore comprises a 5′ homology arm upstream of the first polynucleotide, and a 3′ homology arm downstream of the first polynucleotide. The homology arms of the first donor polynucleotide are typically similar and/or identical to the homology arms of the LPPs into which the first polynucleotide is integrated into.

Second donor polynucleotide: The second donor polynucleotide comprises a second polynucleotide which encodes a second polypeptide of interest. The second donor polynucleotide furthermore comprises a 5′ homology arm upstream of the second polynucleotide, and a 3′ homology arm downstream of the second polynucleotide. The homology arms of the second donor polynucleotide are typically similar and/or identical to the homology arms of the LPPs into which the second polynucleotide is integrated into.

First temperature: The first temperature allows efficient nuclease-based cutting of one or more protospacers which are complementary to one or more first guide RNA. The first temperature typically also allows cultivation of the host cell, but may differ from the host cell's optimal cultivation temperature.

Second guide RNA: The second guide RNA is a guide RNA that forms an editing complex most efficiently at the second temperature, and forms an editing complex less efficiently at other temperatures.

Also, the second guide RNA comprises a spacer sequence which may be complementary to any of the first, second, third, fourth or fifth protospacers. The second guide RNA may be a 1st second guide RNA, a 2nd second guide RNA, 3^{rd} second guide RNA, 4^{rd} second guide RNA, and/or a 5^{th} second guide RNA.

Second temperature: The second temperature allows efficient nuclease-based cutting of one or more protospacers which are complementary to one or more second guide RNA. The second temperature typically also allows cultivation of the host cell, but may differ from the host cell's optimal cultivation temperature. Typically, the second temperature differs from the first temperature by at least 0.5 degrees Celsius.

Third guide RNA: The third guide RNA is a guide RNA that forms an editing complex most efficiently at the third temperature, and forms an editing complex less efficiently at other temperatures. Also, the third guide RNA comprises a spacer sequence which may be complementary to any of the first, second, third, fourth or fifth protospacers. The third guide RNA may be a 1st third guide RNA, a 2nd third guide RNA, 3^{rd} third guide RNA, 4^{rd} third guide RNA, and/or a 5^{th} third guide RNA.

Third temperature: The third temperature allows efficient nuclease-based cutting of one or more protospacers which are complementary to one or more third guide RNA. The third temperature typically also allows cultivation of the host cell, but may differ from the host cell's optimal cultivation temperature.

Typically, the third temperature differs from the first temperature and the second temperature by at least 0.5 degrees Celsius.

Fourth guide RNA: The fourth guide RNA is a guide RNA that forms an editing complex most efficiently at the fourth temperature, and forms an editing complex less efficiently at other temperatures. Also, the fourth guide RNA comprises a spacer sequence which may be complementary to any of the first, second, third, fourth or fifth protospacers. The fourth guide RNA may be a 1st fourth guide RNA, a 2nd fourth guide RNA, 3^{rd} fourth guide RNA, 4^{rd} fourth guide RNA, and/or a 5^{th} fourth guide RNA.

Fourth temperature: The fourth temperature allows efficient nuclease-based cutting of one or more protospacers which are complementary to one or more fourth guide RNA. The fourth temperature typically also allows cultivation of the host cell, but may differ from the host cell's optimal cultivation temperature. Typically, the fourth temperature differs from the first temperature, the second temperature, and the third temperature by at least 0.5 degrees Celsius.

Fifth guide RNA: The fifth guide RNA is a guide RNA that forms an editing complex most efficiently at the fifth temperature, and forms an editing complex less efficiently at other temperatures. Also, the fifth guide RNA comprises a spacer sequence which may be complementary to any of the first, second, third, fourth or fifth protospacers. The fifth third guide RNA may be a 1st fifth guide RNA, a 2nd fifth guide RNA, 3^{rd} fifth guide RNA, 4^{rd} fifth guide RNA, and/or a 5^{th} fifth guide RNA.

Fifth temperature: The fifth temperature allows efficient nuclease-based cutting of one or more protospacers which are complementary to one or more fifth guide RNA. The fifth temperature typically also allows cultivation of the host cell, but may differ from the host cell's optimal cultivation temperature. Typically, the fifth temperature differs from the first temperature, the second temperature, the third temperature, and the fourth temperature by at least 0.5 degrees Celsius.

Homology directed repair: Homology-directed repair is a process where DNA double-strand break is repaired by homologous recombination using a DNA template. The DNA template may be a donor polynucleotide, or a DNA sequence previously integrated into an LPP. The repair mechanism uses the homology arms to integrate the DNA template into the broken target site.

Deficient NHEJ mechanism: Besides homology directed repair, double-strand breaks may also be repaired by a non-homologous end-joining (NHEJ) mechanism. For cells where the NHEJ mechanism has been inactivated, e.g. by mutation or gene deletions, the rate of homology directed repair can be increased.

Empty LPP: The term empty LPP or empty landing pad means an LPP sequence which does not comprise an integrated polynucleotide of interest. Preferably, the empty LPP has not been cut or targeted with a guide RNA previously. Such empty LPP may be subject for homology directed gene integration either with the donor polynucleotide (=during step c)), or with a polynucleotide of interest previously integrated into another LPP (=during step h)).

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: An “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Fusion polypeptide: The term “fusion polypeptide” is a polypeptide in which one polypeptide is fused at the N-terminus and/or the C-terminus of a polypeptide of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Introduced: The term “introduced” in the context of inserting a nucleic acid sequence into a cell, means “transfection”, “transformation” or “transduction,” as known in the art.

Isolated: The term “isolated” means a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

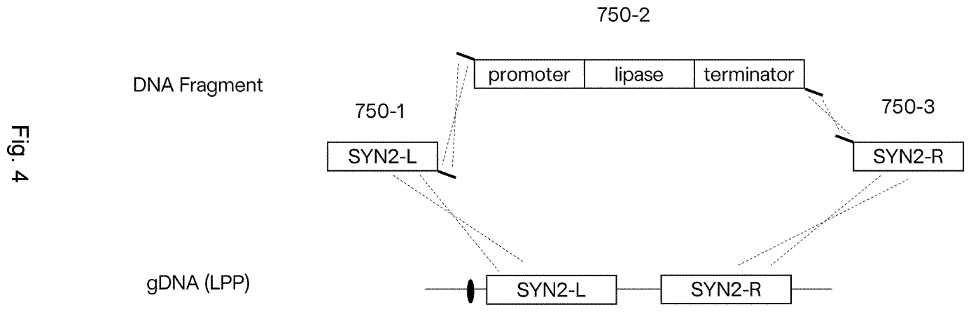

PCR fragments: The term “PCR fragments” means double stranded DNA generated by PCR, comprising overlaps at the 3′ and/or 5′ ends suitable for homologous recombination with one or more other PCR fragments. The homologous recombination takes usually place in the host cell after transformation of the PCR fragments, wherein the PCR fragments are integrated into one or more LPP. Examples for PCR fragments are disclosed in Example 11 and FIG. 4. In a preferred embodiment, a set of PCR fragments comprises a 5′ homology arm PCR fragment, a 3′ homology arm PCR fragment, and a PCR fragment comprising the polynucleotide of interest, preferably the polynucleotide of interest is linked to a promoter and a terminator.

Purified: The term “purified” means a nucleic acid, polypeptide or cell that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

In one aspect, the term “purified” as used herein refers to the polypeptide or cell being essentially free from components (especially insoluble components) from the production organism. In other aspects. the term “purified” refers to the polypeptide being essentially free of insoluble components (especially insoluble components) from the native organism from which it is obtained. In one aspect, the polypeptide is separated from some of the soluble components of the organism and culture medium from which it is recovered. The polypeptide may be purified (i.e., separated) by one or more of the unit operations filtration, precipitation, or chromatography.

Accordingly, the polypeptide may be purified such that only minor amounts of other proteins, in particular, other polypeptides, are present. The term “purified” as used herein may refer to removal of other components, particularly other proteins and most particularly other enzymes present in the cell of origin of the polypeptide. The polypeptide may be “substantially pure”, i.e., free from other components from the organism in which it is produced, e.g., a host organism for recombinantly produced polypeptide. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein. a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The polypeptide of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, polypeptide or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g. depth filtration (by use of filter aids or packed filter medias, cloth filtration in chamber filters, rotary-drum filtration, drum filtration, rotary vacuum-drum filters, candle filters, horizontal leaf filters or similar, using sheet or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hydro cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows: (Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows: (Identical Deoxyribonucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### DETAILED DESCRIPTION OF THE INVENTION

### Host Cells

In a first aspect, the present invention relates to a host cell comprising in its genome at least two landing pad polynucleotide (LPP) sequences, each LPP sequence comprising in 5′ to 3′ direction:
a 5′ homology arm,
two or more protospacer sequences, and
a 3′ homology arm,
wherein the polynucleotide sequence of the 5′ homology arm is identical for at least two LPP sequences, and
wherein the polynucleotide sequence of the 3′ homology arm is identical for at least two LPP sequences.

In one embodiment, at least one protospacer of the at least two protospacers is present in at least two LPP.

In one embodiment, the cell comprises in its genome at least three LPP, wherein at least one LPP comprises second 3′ and second 5′ homology arms having less than 100% sequence identity to the 3′ and 5′ homology arms of the at least two LPP, respectively, e.g., less than 99%, less than 98%, less than 97%, less than 96%, less than 95%, less than 90%, less thank 85%, less than 80%, less than 75%, less than 70%, or less than 65%.

In one embodiment, the polynucleotide sequence of the 5′ homology arm is identical for all of the at least two LPP sequences.

In one embodiment, the polynucleotide sequence of the 3′ homology arm is identical for all of the at least two LPP sequences.

In one embodiment, wherein the 3′ homology arms and the 5′ homology arms are heterologous to the host cell.

In one embodiment, the 3′ homology arms and the 5′ homology arms are synthetic polynucleotide sequences.

In one embodiment, at least one LPP is located on the coding strand (3′ to 5′ direction), and at least one LPP is located on the template strand (5′ to 3′ direction).

In one embodiment, at least two LPPs are located on the coding strand (3′ to 5′ direction), and at least two LPPs are located on the template strand (5′ to 3′ direction).

In one embodiment, each LPP comprises two protospacer sequences, such as a first protospacer, and a second protospacer.

In one embodiment, each LPP comprises three different protospacer sequences, such as a first protospacer, a second protospacer, and a third protospacer.

In one embodiment, each LPP comprises four different protospacer sequences, such as a first protospacer, a second protospacer, a third protospacer, and a fourth protospacer.

In one embodiment, each LPP comprises five different protospacer sequences, such as a first protospacer, a second protospacer, a third protospacer, a fourth protospacer, and a fifth protospacer.

In one embodiment, the first protospacer has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the second protospacer, to the polynucleotide sequence of the third protospacer, to the polynucleotide sequence of the fourth protospacer, and to the polynucleotide sequence of the fifth protospacer.

In one embodiment, the second protospacer has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the first protospacer, to the polynucleotide sequence of the third protospacer, to the polynucleotide sequence of the fourth protospacer, and to the polynucleotide sequence of the fifth protospacer.

In one embodiment, the third protospacer has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the first protospacer, to the polynucleotide sequence of the second protospacer, to the polynucleotide sequence of the fourth protospacer, and to the polynucleotide sequence of the fifth protospacer.

In one embodiment, the fourth protospacer has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the first protospacer, to the polynucleotide sequence of the second protospacer, to the polynucleotide sequence of the third protospacer, and to the polynucleotide sequence of the fifth protospacer.

In one embodiment, wherein the fifth protospacer has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the first protospacer, to the polynucleotide sequence of the second protospacer, to the polynucleotide sequence of the third protospacer, and to the polynucleotide sequence of the fourth protospacer.

In one embodiment, each LPP comprises more than five different protospacer sequences.

In one embodiment, one or more of the at least two protospacer sequences is heterologous to the host cell.

In one embodiment, all of the at least two protospacer sequences are heterologous to the host cell.

In one embodiment, one or more of the at least two protospacer sequences is synthetic.

In one embodiment, all of the at least two protospacer sequences is synthetic.

In one embodiment, each protospacer sequence has a length of at least 15 nt, at least 20 nt, or at least 25 nt, preferably each protospacer has a length of 20-30 nt.

In one embodiment, the host cell comprises two LPP sequences, three LPP sequences, four LPP sequences, 5 LPP sequences, 6 LPP sequences, 7 LPP sequences, 8 LPP sequences, 9 LPP sequences, 10 LPP sequences, 11 LPP sequences, 12 LPP sequences, 13 LPP sequences, 14 LPP sequences, 15 LPP sequences, 16 LPP sequences, or more than 16 LPP sequences.

In one embodiment, the host cell comprises 2-8 LPP sequences.

In one embodiment, the host cell comprises at least two chromosomes, at least four chromosomes, at least six chromosomes or at least 8 chromosomes.

In one embodiment, one or more of the host cell chromosomes comprises a total of 1 LPP per chromosome.

In one embodiment, two or more chromosomes comprise a total of 1 LPP per chromosome.

In one embodiment, three chromosomes comprise a total of 1 LPP per chromosome.

In one embodiment, four chromosomes comprise a total of 1 LPP per chromosome.

In one embodiment, five chromosomes comprise a total of 1 LPP per chromosome.

In one embodiment, six chromosomes comprise a total of 1 LPP per chromosome.

In one embodiment, 7 chromosomes comprise a total of 1 LPP per chromosome.

In one embodiment, 8 chromosomes comprise a total of 1 LPP per chromosome.

In one embodiment, each chromosome comprises a total of 1 LPP per chromosome.

In one embodiment, LPP is located on the short arm of a chromosome.

In one embodiment, the LPP is located on the long arm of a chromosome.

In one embodiment, one or more of the chromosomes comprises a total of 2 LPP per chromosome.

In one embodiment, two or more chromosomes comprise a total of 2 LPP per chromosome.

In one embodiment, three chromosomes comprise a total of 2 LPP per chromosome.

In one embodiment, four chromosomes comprise a total of 2 LPP per chromosome.

In one embodiment, five chromosomes comprise a total of 2 LPP per chromosome.

In one embodiment, six chromosomes comprise a total of 2 LPP per chromosome.

In one embodiment, wherein 7 chromosomes comprise a total of 2 LPP per chromosome.

In one embodiment, 8 chromosomes comprise a total of 2 LPP per chromosome.

In one embodiment, each chromosome comprises a total of 2 LPP per chromosome.

In one embodiment, for each of the one or more chromosomes, the 2 LPP are located on opposite locations of the centromere of the chromosome.

In one embodiment, one LPP is located on the short arm, and one LPP is located on the long arm.

In one embodiment, one or more of the chromosomes comprises a total of 3 LPP per chromosome.

In one embodiment, wherein two or more chromosomes comprise a total of 3 LPP per chromosome.

In one embodiment, three chromosomes comprise a total of 3 LPP per chromosome.

In one embodiment, four chromosomes comprise a total of 3 LPP per chromosome.

In one embodiment, five chromosomes comprise a total of 3 LPP per chromosome.

In one embodiment, six chromosomes comprise a total of 3 LPP per chromosome.

In one embodiment, 7 chromosomes comprise a total of 3 LPP per chromosome.

In one embodiment, 8 chromosomes comprise a total of 3 LPP per chromosome.

In one embodiment, each chromosome comprises a total of 3 LPP per chromosome.

In one embodiment, comprising 3 LPP per chromosome, the ratio of numbers of LPP between short arm and long arm is selected from the list of 3:0, 0:3, 2:1, and 1:2.

In one embodiment, one or more of the chromosomes comprises a total of 4 LPP per chromosome.

In one embodiment, two or more chromosomes comprise a total of 4 LPP per chromosome.

In one embodiment, three chromosomes comprise a total of 4 LPP per chromosome.

In one embodiment, four chromosomes comprise a total of 4 LPP per chromosome.

In one embodiment, five chromosomes comprise a total of 4 LPP per chromosome.

In one embodiment, six chromosomes comprise a total of 4 LPP per chromosome.

In one embodiment, 7 chromosomes comprise a total of 4 LPP per chromosome.

In one embodiment, 8 chromosomes comprise a total of 4 LPP per chromosome.

In one embodiment, each chromosome comprises a total of 4 LPP per chromosome.

In one embodiment, for 4 LPP per chromosome, the ratio of numbers of LPP between short arm and long arm is selected from the list of 4:0, 0:4, 3:1, 1:3, 2:2, 2:1, and 1:2.

In one embodiment, the host cell is deficient of a non-homologous end joining (NHEJ) mechanism. The deficiency in the NHEJ mechanism increases the efficiency of homology directed repair and thus increases the efficiency of DNA integration using the method of the second aspect. Furthermore, NHEJ-deficiency in host cells increases the stability of the genome, also when there are empty LPPs present in the host cell genome.

In one embodiment, each 3′ homology arm and each 5′ homology arm has a length of at least 10 nt, at least 20 nt, at least 30 nt, at least 50 nt, at least 100 nt, 200 nucleotides (nt), at least 300 nt, at least 400 nt, at least 500 nt, at least 600 nt, at least 700 nt, at least 800 nt, at least 900 nt, or at least 1000 nt.

In one embodiment, each 3′ homology arm and each 5′ homology arm has a length of at least 800 nt, at least 900 nt, or at least 1000 nt.

In one embodiment, each 3′ homology arm and each 5′ homology arm has a length of at least 100 nt.

In one embodiment, one or more LPP of the at least two LPP comprise a selectable marker, and/or a counter-selectable marker.

In one embodiment, 2 or more LPP of the at least two LPP comprise a selectable marker, and/or a counter-selectable marker.

In one embodiment, 3 or more LPP of the at least two LPP comprise a selectable marker, and/or a counter-selectable marker.

In one embodiment, 4 or more LPP of the at least two LPP comprise a selectable marker, and/or a counter-selectable marker.

In one embodiment, 5 or more LPP of the at least two LPP comprise a selectable marker, and/or a counter-selectable marker.

In one embodiment, 6 or more LPP of the at least two LPP comprise a selectable marker, and/or a counter-selectable marker.

In one embodiment, 7 or more LPP of the at least two LPP comprise a selectable marker, and/or a counter-selectable marker.

In one embodiment, 8 or more LPP of the at least two LPP comprise a selectable marker, and/or a counter-selectable marker.

In one embodiment, the selectable marker comprises a marker selected from the list of a pyrG gene, a fcy1 gene, an adeA gene, an adeB gene, a metF gene, a metG gene, a metH gene, a lysF gene, a trpC gene, a sC gene, an argB gene, and a gene encoding a fluorescent protein, such as a GFP, YFP, CFP, DsRed, or a eqPF611.

In one embodiment, the counter-selectable marker comprises a marker selected from the list of a pyrG gene, a fcy1 gene, an adeA gene, an adeB gene, a metF gene, a metG gene, a metH gene, a lysF gene, a trpC gene, a sC gene, an argB gene, and a gene encoding a fluorescent protein, such as a GFP, YFP, CFP, DsRed, or a eqPF611.

In one embodiment, none of the at least two LPP comprises a selectable marker, and/or a counter-selectable marker.

In one embodiment, each DNA strain complementary to the two or more protospacers sequences comprises a PAM sequence (protospacer adjacent motif).

In one embodiment, the PAM sequence is selected from the list of “NGG”, “NGRRT”, “NGRRN”, “NNNNGATT”, “NNNNRYAC”, “NNAGAAW”, “TTTV”, “TTTC”, “TTTN”, “TTN”, “ATTN”, “GTTN”, and “CTTN” (“N” being any nucleotide, “Y”=C or T, “V”=A or C or G, “W”=A or T, “R”=A or G), preferably the PAM sequence is TTTN, more preferably the PAM sequence is TTTC.

In one embodiment, the first, second, third, fourth or fifth protospacer sequence is selected from a sequence having a sequence identity of at least 80%, at least 85%, at least 90%, at least 95%, at least 96%, at least 97%, at least 98%, at least 99%, or 100% to SEQ ID NO: 3, SEQ ID NO: 4, SEQ ID NO: 5, SEQ ID NO: 6, SEQ ID NO: 7, SEQ ID NO: 8, SEW ID NO: 9, SEQ ID NO: 10, SEQ ID NO: 11, SEQ ID NO: 12, or SEQ ID NO: 13.

In one embodiment, the 5′ homology arm sequences comprises or consists of a polynucleotide sequence having a sequence identity of at least 80%, at least 85%, at least 90%, at least 95%, at least 96%, at least 97%, at least 98%, at least 99%, or 100% to SEQ ID NO: 1 or SEQ ID NO: 2.

In one embodiment, the 5′ homology arm sequence comprises or consists of SEQ ID NO: 1 or SEQ ID NO: 2.

In one embodiment, the 3′ homology arm sequence comprises or consists of SEQ ID NO: 1 or SEQ ID NO: 2.

In one embodiment, the 3′ homology arm sequences comprises or consists of a polynucleotide sequence having a sequence identity of at least 80%, at least 85%, at least 90%, at least 95%, at least 96%, at least 97%, at least 98%, at least 99%, or 100% to SEQ ID NO: 1 or SEQ ID NO: 2.

In one embodiment, wherein each of the at least two LPP comprises a first protospacer sequence with a polynucleotide sequence identical for each first protospacer of each at least two LPP. When the host cell is targeted with a nuclease and gRNA complementary to the first protospacer, each LPP is subject for a double-strand break, and thus the gene of interest can be integrated into each of the LPPs of the host cell.

In one embodiment, each of the at least two LPP comprises a second protospacer sequence with a polynucleotide sequence which is unique for each second protospacer of each at least two LPP. When the host cell is targeted with a nuclease and gRNA complementary to the second protospacer, only one LPP is subject for a double-strand break, and thus the gene of interest can be integrated into only one LPP of the LPPs of the host cell.

In one embodiment, each of the at least two LPP comprises a third protospacer sequence with a polynucleotide sequence which is identical for each third polynucleotide within a third subset of the at least two LPPs. When the host cell is targeted with a nuclease and gRNA complementary to the third protospacer, the third subset of LPPs is subject for a double-strand break, and thus the gene of interest can be integrated into each of the LPPs of the third subset.

In one embodiment, the third subset consists of 2 LPP sequences, 3 LPP sequences, 4 LPP sequences, 5 LPP sequences, 6 LPP sequences, 7 LPP sequences, 8 LPP sequences, 9 LPP sequences, 10 LPP, sequences, 11 LPP sequences, 12 LPP sequences, 13 LPP sequences, 14 LPP sequences, or 15 LPP sequences.

In one embodiment, each of the at least two LPP comprises a fourth protospacer sequence with a polynucleotide sequence which is identical for each fourth polynucleotide within a fourth subset of the at least two LPPs. When the host cell is targeted with a nuclease and gRNA complementary to the fourth protospacer, the fourth subset of LPPs is subject for a double-strand break, and thus the gene of interest can be integrated into each of the LPPs of the fourth subset.

In one embodiment, the fourth subset consists of 2 LPP sequences, 3 LPP sequences, 4 LPP sequences, 5 LPP sequences, 6 LPP sequences, 7 LPP sequences, 8 LPP sequences, 9 LPP sequences, 10 LPP, sequences, 11 LPP sequences, 12 LPP sequences, 13 LPP sequences, 14 LPP sequences, or 15 LPP sequences.

In one embodiment, each of the at least two LPP comprises a fifth protospacer sequence with a polynucleotide sequence which is identical for each fifth protospacer within a fifth subset of the at least two LPPs. When the host cell is targeted with a nuclease and gRNA complementary to the fifth protospacer, the fifth subset of LPPs is subject for a double-strand break, and thus the gene of interest can be integrated into each of the LPPs of the fifth subset.

In one embodiment, the fifth subset consists of 2 LPP sequences, 3 LPP sequences, 4 LPP sequences, 5 LPP sequences, 6 LPP sequences, 7 LPP sequences, 8 LPP sequences, 9 LPP sequences, 10 LPP, sequences, 11 LPP sequences, 12 LPP sequences, 13 LPP sequences, 14 LPP sequences, or 15 LPP sequences.

In one embodiment, the host cell comprises three LPP sequences, and wherein the third, fourth, or fifth protospacer sequence is identical for two of the three LPPs.

In one embodiment, the host cell comprises four LPP sequences, and wherein the third, fourth, or fifth protospacer sequence is identical for two of the four LPPs.

In one embodiment, the host cell comprises four LPP sequences, and wherein the third, fourth, or fifth protospacer sequence is identical for three of the four LPPs.

In one embodiment, the host cell comprises five LPP sequences, and wherein the third, fourth, or fifth protospacer sequence is identical for two of the five LPPs.

In one embodiment, the host cell comprises five LPP sequences, and wherein the third, fourth, or fifth protospacer sequence is identical for three of the five LPPs.

In one embodiment, the host cell comprises five LPP sequences, and wherein the third, fourth, or fifth protospacer sequence is identical for four of the five LPPs.

In one embodiment, the host cell comprises six LPP sequences, and wherein the third, fourth, or fifth protospacer sequence is identical for two of the six LPPs.

In one embodiment, the host cell comprises six LPP sequences, and wherein the third, fourth, or fifth protospacer sequence is identical for three of the six LPPs.

In one embodiment, the host cell comprises six LPP sequences, and wherein the third, fourth, or fifth protospacer sequence is identical for four of the six LPPs.

In one embodiment, the host cell comprises six LPP sequences, and wherein the third, fourth, or fifth protospacer sequence is identical for five of the six LPPs.

In one embodiment, the host cell comprises 7 LPP sequences, and wherein the third, fourth, or fifth protospacer sequence is identical for two of the 7 LPPs.

In one embodiment, the host cell comprises 7 LPP sequences, and wherein the third, fourth, or fifth protospacer sequence is identical for three of the 7 LPPs.

In one embodiment, the host cell comprises 7 LPP sequences, and wherein the third, fourth, or fifth protospacer sequence is identical for four of the 7 LPPs.

In one embodiment, the host cell comprises 7 LPP sequences, and wherein the third, fourth, or fifth protospacer sequence is identical for five of the 7 LPPs.

In one embodiment, the host cell comprises 7 LPP sequences, and wherein the third, fourth, or fifth protospacer sequence is identical for six of the 7 LPPs.

In one embodiment, the host cell comprises 8 LPP sequences, and wherein the third, fourth, or fifth protospacer sequence is identical for two of the 8 LPPs.

In one embodiment, the host cell comprises 8 LPP sequences, and wherein the third, fourth, or fifth protospacer sequence is identical for three of the 8 LPPs.

In one embodiment, the host cell comprises 8 LPP sequences, and wherein the third, fourth, or fifth protospacer sequence is identical for four of the 8 LPPs.

In one embodiment, the host cell comprises 8 LPP sequences, and wherein the third, fourth, or fifth protospacer sequence is identical for five of the 8 LPPs.

In one embodiment, the host cell comprises 8 LPP sequences, and wherein the third, fourth, or fifth protospacer sequence is identical for six of the 8 LPPs.

In one embodiment, the host cell comprises 8 LPP sequences, and wherein the third, fourth, or fifth protospacer sequence is identical for 7 of the 8 LPPs.

In one embodiment, the host cell comprises 9 LPP sequences, and wherein the third, fourth, or fifth protospacer sequence is identical for two of the 9 LPPs.

In one embodiment, the host cell comprises 9 LPP sequences, and wherein the third, fourth, or fifth protospacer sequence is identical for three of the 9 LPPs.

In one embodiment, the host cell comprises 9 LPP sequences, and wherein the third, fourth, or fifth protospacer sequence is identical for four of the 9 LPPs.

In one embodiment, the host cell comprises 9 LPP sequences, and wherein the third, fourth, or fifth protospacer sequence is identical for five of the 9 LPPs.

In one embodiment, the host cell comprises 9 LPP sequences, and wherein the third, fourth, or fifth protospacer sequence is identical for six of the 9 LPPs.

In one embodiment, the host cell comprises 9 LPP sequences, and wherein the third, fourth, or fifth protospacer sequence is identical for 7 of the 9 LPPs.

In one embodiment, the host cell comprises 9 LPP sequences, and wherein the third, fourth, or fifth protospacer sequence is identical for 8 of the 9 LPPs.

In one embodiment, the host cell comprises 10 LPP sequences, and wherein the third, fourth, or fifth protospacer sequence is identical for two of the 10 LPPs.

In one embodiment, the host cell comprises 10 LPP sequences, and wherein the third, fourth, or fifth protospacer sequence is identical for three of the 10 LPPs.

In one embodiment, the host cell comprises 10 LPP sequences, and wherein the third, fourth, or fifth protospacer sequence is identical for four of the 10 LPPs.

In one embodiment, the host cell comprises 10 LPP sequences, and wherein the third, fourth, or fifth protospacer sequence is identical for five of the 10 LPPs.

In one embodiment, the host cell comprises 10 LPP sequences, and wherein the third, fourth, or fifth protospacer sequence is identical for six of the 10 LPPs.

In one embodiment, the host cell comprises 10 LPP sequences, and wherein the third, fourth, or fifth protospacer sequence is identical for 7 of the 10 LPPs.

In one embodiment, the host cell comprises 10 LPP sequences, and wherein the third, fourth, or fifth protospacer sequence is identical for 8 of the 10 LPPs.

In one embodiment, the host cell comprises 10 LPP sequences, and wherein the third, fourth, or fifth protospacer sequence is identical for 9 of the 10 LPPs.

In one embodiment, the host cell comprises 11 LPP sequences, and wherein the third, fourth, or fifth protospacer sequence is identical for two of the 11 LPPs.

In one embodiment, the host cell comprises 11 LPP sequences, and wherein the third, fourth, or fifth protospacer sequence is identical for three of the 11 LPPs.

In one embodiment, the host cell comprises 11 LPP sequences, and wherein the third, fourth, or fifth protospacer sequence is identical for four of the 11 LPPs.

In one embodiment, the host cell comprises 11 LPP sequences, and wherein the third, fourth, or fifth protospacer sequence is identical for five of the 11 LPPs.

In one embodiment, the host cell comprises 11 LPP sequences, and wherein the third, fourth, or fifth protospacer sequence is identical for six of the 11 LPPs.

In one embodiment, the host cell comprises 11 LPP sequences, and wherein the third, fourth, or fifth protospacer sequence is identical for 7 of the 11 LPPs.

In one embodiment, the host cell comprises 11 LPP sequences, and wherein the third, fourth, or fifth protospacer sequence is identical for 8 of the 11 LPPs.

In one embodiment, the host cell comprises 11 LPP sequences, and wherein the third, fourth, or fifth protospacer sequence is identical for 9 of the 11 LPPs.

In one embodiment, the host cell comprises 11 LPP sequences, and wherein the third, fourth, or fifth protospacer sequence is identical for 10 of the 11 LPPs.

In one embodiment, the host cell comprises 12 LPP sequences, and wherein the third, fourth, or fifth protospacer sequence is identical for two of the 12 LPPs.

In one embodiment, the host cell comprises 12 LPP sequences, and wherein the third, fourth, or fifth protospacer sequence is identical for three of the 12 LPPs.

In one embodiment, the host cell comprises 12 LPP sequences, and wherein the third, fourth, or fifth protospacer sequence is identical for four of the 12 LPPs.

In one embodiment, the host cell comprises 12 LPP sequences, and wherein the third, fourth, or fifth protospacer sequence is identical for five of the 12 LPPs.

In one embodiment, the host cell comprises 12 LPP sequences, and wherein the third, fourth, or fifth protospacer sequence is identical for six of the 12 LPPs.

In one embodiment, the host cell comprises 12 LPP sequences, and wherein the third, fourth, or fifth protospacer sequence is identical for 7 of the 12 LPPs.

In one embodiment, the host cell comprises 12 LPP sequences, and wherein the third, fourth, or fifth protospacer sequence is identical for 8 of the 12 LPPs.

In one embodiment, the host cell comprises 12 LPP sequences, and wherein the third, fourth, or fifth protospacer sequence is identical for 9 of the 12 LPPs.

In one embodiment, the host cell comprises 12 LPP sequences, and wherein the third, fourth, or fifth protospacer sequence is identical for 10 of the 12LPPs.

In one embodiment, the host cell comprises 12 LPP sequences, and wherein the third, fourth, or fifth protospacer sequence is identical for 11 of the 12 LPPs.

In one embodiment, the host cell comprises 13 LPP sequences, and wherein the third, fourth, or fifth protospacer sequence is identical for two of the 13 LPPs.

In one embodiment, the host cell comprises 13 LPP sequences, and wherein the third, fourth, or fifth protospacer sequence is identical for three of the 13 LPPs.

In one embodiment, the host cell comprises 13 LPP sequences, and wherein the third, fourth, or fifth protospacer sequence is identical for four of the 13 LPPs.

In one embodiment, the host cell comprises 13 LPP sequences, and wherein the third, fourth, or fifth protospacer sequence is identical for five of the 13 LPPs.

In one embodiment, the host cell comprises 13 LPP sequences, and wherein the third, fourth, or fifth protospacer sequence is identical for six of the 13 LPPs.

In one embodiment, the host cell comprises 13 LPP sequences, and wherein the third, fourth, or fifth protospacer sequence is identical for 7 of the 13 LPPs.

In one embodiment, the host cell comprises 13 LPP sequences, and wherein the third, fourth, or fifth protospacer sequence is identical for 8 of the 13 LPPs.

In one embodiment, the host cell comprises 13 LPP sequences, and wherein the third, fourth, or fifth protospacer sequence is identical for 9 of the 13 LPPs.

In one embodiment, the host cell comprises 13 LPP sequences, and wherein the third, fourth, or fifth protospacer sequence is identical for 10 of the 13 LPPs.

In one embodiment, the host cell comprises 13 LPP sequences, and wherein the third, fourth, or fifth protospacer sequence is identical for 11 of the 13 LPPs.

In one embodiment, the host cell comprises 13 LPP sequences, and wherein the third, fourth, or fifth protospacer sequence is identical for 12 of the 13 LPPs.

In one embodiment, the host cell comprises 14 LPP sequences, and wherein the third, fourth, or fifth protospacer sequence is identical for two of the 14 LPPs.

In one embodiment, the host cell comprises 14 LPP sequences, and wherein the third, fourth, or fifth protospacer sequence is identical for three of the 14 LPPs.

In one embodiment, the host cell comprises 14 LPP sequences, and wherein the third, fourth, or fifth protospacer sequence is identical for four of the 14 LPPs.

In one embodiment, the host cell comprises 14 LPP sequences, and wherein the third, fourth, or fifth protospacer sequence is identical for five of the 14 LPPs.

In one embodiment, the host cell comprises 14 LPP sequences, and wherein the third, fourth, or fifth protospacer sequence is identical for six of the 14 LPPs.

In one embodiment, the host cell comprises 14 LPP sequences, and wherein the third, fourth, or fifth protospacer sequence is identical for 7 of the 14 LPPs.

In one embodiment, the host cell comprises 14 LPP sequences, and wherein the third, fourth, or fifth protospacer sequence is identical for 8 of the 14 LPPs.

In one embodiment, the host cell comprises 14 LPP sequences, and wherein the third, fourth, or fifth protospacer sequence is identical for 9 of the 14 LPPs.

In one embodiment, the host cell comprises 14 LPP sequences, and wherein the third, fourth, or fifth protospacer sequence is identical for 10 of the 14 LPPs.

In one embodiment, the host cell comprises 14 LPP sequences, and wherein the third, fourth, or fifth protospacer sequence is identical for 11 of the 14 LPPs.

In one embodiment, the host cell comprises 14 LPP sequences, and wherein the third, fourth, or fifth protospacer sequence is identical for 12 of the 14 LPPs.

In one embodiment, the host cell comprises 14 LPP sequences, and wherein the third, fourth, or fifth protospacer sequence is identical for 13 of the 14 LPPs.

In one embodiment, the host cell comprises 15 LPP sequences, and wherein the third, fourth, or fifth protospacer sequence is identical for two of the 15 LPPs.

In one embodiment, the host cell comprises 15 LPP sequences, and wherein the third, fourth, or fifth protospacer sequence is identical for three of the 15 LPPs.

In one embodiment, the host cell comprises 15 LPP sequences, and wherein the third, fourth, or fifth protospacer sequence is identical for four of the 15 LPPs.

In one embodiment, the host cell comprises 15 LPP sequences, and wherein the third, fourth, or fifth protospacer sequence is identical for five of the 15 LPPs.

In one embodiment, the host cell comprises 15 LPP sequences, and wherein the third, fourth, or fifth protospacer sequence is identical for six of the 15 LPPs.

In one embodiment, the host cell comprises 15 LPP sequences, and wherein the third, fourth, or fifth protospacer sequence is identical for 7 of the 15 LPPs.

In one embodiment, the host cell comprises 15 LPP sequences, and wherein the third, fourth, or fifth protospacer sequence is identical for 8 of the 15 LPPs.

In one embodiment, the host cell comprises 15 LPP sequences, and wherein the third, fourth, or fifth protospacer sequence is identical for 9 of the 15 LPPs.

In one embodiment, the host cell comprises 15 LPP sequences, and wherein the third, fourth, or fifth protospacer sequence is identical for 10 of the 15 LPPs.

In one embodiment, the host cell comprises 15 LPP sequences, and wherein the third, fourth, or fifth protospacer sequence is identical for 11 of the 15 LPPs.

In one embodiment, the host cell comprises 15 LPP sequences, and wherein the third, fourth, or fifth protospacer sequence is identical for 12 of the 15 LPPs.

In one embodiment, the host cell comprises 15 LPP sequences, and wherein the third, fourth, or fifth protospacer sequence is identical for 13 of the 15 LPPs.

In one embodiment, the host cell comprises 15 LPP sequences, and wherein the third, fourth, or fifth protospacer sequence is identical for 14 of the 15 LPPs.

In one embodiment, the host cell comprises 16 LPP sequences, and wherein the third, fourth, or fifth protospacer sequence is identical for two of the 16 LPPs.

In one embodiment, the host cell comprises 16 LPP sequences, and wherein the third, fourth, or fifth protospacer sequence is identical for three of the 16 LPPs.

In one embodiment, the host cell comprises 16 LPP sequences, and wherein the third, fourth, or fifth protospacer sequence is identical for four of the 16 LPPs.

In one embodiment, the host cell comprises 16 LPP sequences, and wherein the third, fourth, or fifth protospacer sequence is identical for five of the 16 LPPs.

In one embodiment, the host cell comprises 16 LPP sequences, and wherein the third, fourth, or fifth protospacer sequence is identical for six of the 16 LPPs.

In one embodiment, the host cell comprises 16 LPP sequences, and wherein the third, fourth, or fifth protospacer sequence is identical for 7 of the 16 LPPs.

In one embodiment, the host cell comprises 16 LPP sequences, and wherein the third, fourth, or fifth protospacer sequence is identical for 8 of the 16 LPPs.

In one embodiment, the host cell comprises 16 LPP sequences, and wherein the third, fourth, or fifth protospacer sequence is identical for 9 of the 16 LPPs.

In one embodiment, the host cell comprises 16 LPP sequences, and wherein the third, fourth, or fifth protospacer sequence is identical for 10 of the 16 LPPs.

In one embodiment, the host cell comprises 16 LPP sequences, and wherein the third, fourth, or fifth protospacer sequence is identical for 11 of the 16 LPPs.

In one embodiment, the host cell comprises 16 LPP sequences, and wherein the third, fourth, or fifth protospacer sequence is identical for 12 of the 16 LPPs.

In one embodiment, the host cell comprises 16 LPP sequences, and wherein the third, fourth, or fifth protospacer sequence is identical for 13 of the 16 LPPs.

In one embodiment, the host cell comprises 16 LPP sequences, and wherein the third, fourth, or fifth protospacer sequence is identical for 14 of the 16 LPPs.

In one embodiment, the host cell comprises 16 LPP sequences, and wherein the third, fourth, or fifth protospacer sequence is identical for 15 of the 16 LPPs.

In one embodiment, the host cell is a fungal cell.

In one embodiment, the host cell is a yeast recombinant host cell, e.g., a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

In one embodiment, the host cell is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In one embodiment, the host cell is an Aspergillus cell, preferably an Aspergillus nigercell, or an Aspergillus oryzae cell.

In one embodiment, the host cell is a Trichoderma cell, preferably a Trichderma reesei cell.

In one embodiment, the host cell is isolated.

In one embodiment, the host cell is purified.

In one embodiment, the host cell is a prokaryotic recombinant host cell, e.g., a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, or a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.

In one embodiment, the host cell is a Bacillus cell.

In one embodiment, the host cell is a Bacillus licheniformis cell.

In one embodiment, the host cell is a Bacillus subtilis cell.

In one embodiment, the host cell is a prokaryotic host cell and wherein at least one LPP is located between two selectable and/or counter-selectable marker genes.

In one embodiment, the host cell is a mammalian cell.

In one embodiment, the mammalian cell is selected from the list of a human cell, a mouse cell, a rat cell, a mouse hybridoma cell, a hamster cell (e.g. Chinese hamster ovary cell), and a rat hybridoma cell.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source. The polypeptide can be native or heterologous to the recombinant host cell.

Also, at least one of the one or more control sequences can be heterologous to the polynucleotide encoding the polypeptide of interest. The recombinant host cell may comprise at least two copies, e.g., three, four, five, or more copies of the polynucleotide of the present invention.

The host cell may be any microbial cell useful in the recombinant production of a polypeptide of interest, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

For purposes of this invention, Bacillus classes/genera/species shall be defined as described in Patel and Gupta, 2020, Int. J. Syst. Evol. Microbiol. 70: 406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98: 6289-6294, Choi et al., 2006, J. Microbiol. Methods 64: 391-397, and Donald et al., 2013, J. Bacteriol. 195(11): 2612-2620.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8^{th} edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16: 168 and procedures described in EP 238023, Yelton et al., 1984, Proc. Natl. Acad. Sci. USA 81: 1470-1474, Christensen et al., 1988, Bio/Technology 6: 1419-1422, and Lubertozzi and Keasling, 2009, Biotechn. Advances 27: 53-75. However, any method known in the art for introducing DNA into a fungal host cell can be used, and the DNA can be introduced as linearized or as circular polynucleotide.

The fungal host cell may be a yeast cell. “Yeast” as used herein includes ascosporogenous yeast (Endomycetales), basidiosporogenous yeast, and yeast belonging to the Fungi Imperfecti (Blastomycetes). For purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

### Landing Pad Polynucleotides (LPPs)

The present invention also relates to at least two LPPs present in the host cell genome, as described herein. Each LPP comprising a 5′ homology arm, two or more protospacers, and a 3′ homology arm.

The LPP may be a genomic DNA, a cDNA, a synthetic DNA, or a combination thereof.

In an aspect, the LPP comprises an protospacer array of at least 2 protospacers comprising at least 50 nucleotides (e.g., nucleotides 1 to 50 of SEQ ID NOs: 14-17).

In an aspect, the LPP comprises an protospacer array of at least 3 protospacers comprising at least 75 nucleotides (e.g., nucleotides 1 to 75 of SEQ ID NOs: 14-17).

In an aspect, the LPP comprises an protospacer array of at least 4 protospacers comprising at least 100 nucleotides (e.g., nucleotides 1 to 100 of SEQ ID NOs: 14-17).

In an aspect, the LPP comprises an protospacer array of at least 5 protospacers comprising at least 125 nucleotides (e.g., nucleotides 1 to 125 of SEQ ID NOs: 14-17).

The protospacers of the LPP may also be mutated by introduction of nucleotide substitutions, or by introduction of nucleotide substitutions that may give rise to a different target sequence. The homology arms of the LPP may also be mutated by introduction of nucleotide substitutions, or by introduction of nucleotide substitutions that may give rise to a different homology arm sequence. For a general description of nucleotide substitution, see, e.g., Ford et al., 1991, Protein Expression and Purification 2: 95-107.

In an aspect, the LPP is isolated.

In another aspect, the LPP is purified.

### Nucleic Acid Constructs

The present invention also relates to nucleic acid constructs comprising an LPP, and to nucleic acid constructs comprising a donor polynucleotide of the present invention.

In the donor polynucleotide, the polynucleotide encoding a polypeptide of interest is operably linked to one or more control sequences that direct the expression of the coding sequence in a suitable host cell under conditions compatible with the control sequences.

The donor polynucleotide may be manipulated in a variety of ways to provide for expression of the polypeptide. Manipulation of the polynucleotide prior to its insertion into a vector may be desirable or necessary depending on the expression vector. Techniques for modifying polynucleotides utilizing recombinant DNA methods are well known in the art.

### Promoters

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, supra, and Song et al., 2016, PLOS One 11(7): e0158447.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenböck, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenböck, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8: 423-488.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

### Leader Sequences

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Suitable leaders for bacterial host cells are described by Hambraeus et al., 2000, Microbiology 146(12): 3051-3059, and by Kaberdin and Blasi, 2006, FEMS Microbiol. Rev. 30(6): 967-979.

Preferred leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

### Signal Peptides

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. Any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Freudl, 2018, Microbial Cell Factories 17: 52.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40: 949-955

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

### Propeptides

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. Additionally or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In fungal systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52: 7-23, as well in Balleza et al., 2009, FEMS Microbiol. Rev. 33(1): 133-151.

### Expression Vectors

The present invention also relates to recombinant expression vectors comprising a donor polynucleotide of the present invention, a promoter, and transcriptional and translational stop signals. The various nucleotide, homology arms and control sequences may be joined together to produce a recombinant expression vector that may include one or more convenient restriction sites to allow for insertion or substitution of the donor polynucleotide encoding the polypeptide at the LPPs. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide of interest. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector may contain one or more selectable markers that permit easy selection of transformed, transfected, transduced, or the like cells. A selectable marker is a gene the product of which provides for biocide or viral resistance, resistance to heavy metals, prototrophy to auxotrophs, and the like.

For integration into the host cell genome, the vector may rely on homology arms which facilitate integration via homology-directed repair (HDR).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a donor polynucleotide of the present invention may be inserted into a host cell to increase production of a polypeptide of interest. For example, 2 or 3 or 4 or 5 or more copies are inserted into a host cell.

### Method for Generating Multi-Copy Host Cells

In a second aspect, the present invention relates to a method for generating a multi-copy host cell, comprising the steps of:
a) providing a host cell according to the first aspect,
b) delivering to the host cell a gene editing system comprising:
   a RNA-guided DNA nuclease,
   at least one guide RNA directed to one or more protospacer of the one or more LPP sequences,
   a first donor polynucleotide comprising in 5′ to 3′ direction: a 5′ homology arm, a first polynucleotide encoding a first polypeptide of interest, and a 3′ homology arm, OR
   a set of first PCR fragments comprising a first 5′ homology arm, a first 3′ homology arm, and a first polynucleotide encoding a first polypeptide of interest wherein the 3′ homology arm is identical to the 3′ homology arm of one or more LPP, and wherein the 5′ homology arm is identical to the 5′ homology arm of one or more LPP, and
c) cultivating the host cell at a first temperature allowing one or more first guide RNA of the at least one guide RNA to form a complex with the nuclease at one or more protospacer of one or more LPP, and wherein the complex promotes the homology directed repair by creating a double-strand break at or near the one or more protospacer sequence of the one or more LPP, wherein the homology directed repair comprises insertion of the first donor polynucleotide or set of first PCR fragments at the double-strand break.

In one embodiment, the first polypeptide is heterologous to the host cell.

In one embodiment, the first polynucleotide is operably linked to one or more control sequences that direct the production of the first polypeptide.

In one embodiment, at least one of the one or more control sequences is heterologous to the first polynucleotide.

In one embodiment, the first guide RNA is complementary to one or more first protospacers, wherein during step c) the complex is formed at one or more first protospacer of the one or more LPP, creating a double-strand break at or near the one or more first protospacer.

In one embodiment, the first guide RNA is complementary to one or more second protospacers, wherein during step c) the complex is formed at one or more second protospacer of the one or more LPP, creating a double-strand break at or near the one or more second protospacer.

In one embodiment, the first guide RNA is complementary to one or more third protospacers, wherein during step c) the complex is formed at one or more third protospacer of the one or more LPP, creating a double-strand break at or near the one or more third protospacer.

In one embodiment, the first guide RNA is complementary to one or more fourth protospacers, wherein during step c) the complex is formed at one or more fourth protospacer of the one or more LPP, creating a double-strand break at or near the one or more fourth protospacer.

In one embodiment, the first guide RNA is complementary to one or more fifth protospacers, wherein during step c) the complex is formed at one or more fifth protospacer of the one or more LPP, creating a double-strand break at or near the one or more fifth protospacer.

In one embodiment, during step c) the first temperature allows the one or more first guide RNA to form a complex at one or more protospacers of one LPP, and wherein the complex promotes the homology directed repair by creating a double-strand break at or near the one or more protospacer sequence of the one LPP, wherein the homology directed repair comprises insertion of the first donor polynucleotide or set of first PCR fragments at the double-strand break to provide a host cell with one copy of the first donor polynucleotide in its genome.

In one embodiment, during step c) the first temperature allows the one or more first guide RNA to form a complex at one or more protospacers of two LPP, and wherein the complex promotes the homology directed repair by creating a double-strand break at or near the one or more protospacer sequence of the two LPP, wherein the homology directed repair comprises insertion of the first donor polynucleotide or set of first PCR fragments at the double-strand break to provide a host cell with two copies of the first donor polynucleotide in its genome.

In one embodiment, during step c) the first temperature allows the one or more first guide RNA to form a complex at one or more protospacers of three LPP, and wherein the complex promotes the homology directed repair by creating a double-strand break at or near the one or more protospacer sequence of the three LPP, wherein the homology directed repair comprises insertion of the first donor polynucleotide or set of first PCR fragments at the double-strand break to provide a host cell with three copies of the first donor polynucleotide in its genome.

In one embodiment, during step c) the first temperature allows the one or more first guide RNA to form a complex at one or more protospacers of four LPP, and wherein the complex promotes the homology directed repair by creating a double-strand break at or near the one or more protospacer sequence of the four LPP, wherein the homology directed repair comprises insertion of the first donor polynucleotide or set of first PCR fragments at the double-strand break to provide a host cell with four copies of the first donor polynucleotide in its genome.

In one embodiment, during step c) the first temperature allows the one or more first guide RNA to form a complex at one or more protospacers of five LPP, and wherein the complex promotes the homology directed repair by creating a double-strand break at or near the one or more protospacer sequence of the five LPP, wherein the homology directed repair comprises insertion of the first donor polynucleotide or set of first PCR fragments at the double-strand break to provide a host cell with five copies of the first donor polynucleotide in its genome.

In one embodiment, during step c) the first temperature allows the one or more first guide RNA to form a complex at one or more protospacers of six LPP, and wherein the complex promotes the homology directed repair by creating a double-strand break at or near the one or more protospacer sequence of the six LPP, wherein the homology directed repair comprises insertion of the first donor polynucleotide or set of first PCR fragments at the double-strand break to provide a host cell with six copies of the first donor polynucleotide in its genome.

In one embodiment, during step c) the first temperature allows the one or more first guide RNA to form a complex at one or more protospacers of 7 LPP, and wherein the complex promotes the homology directed repair by creating a double-strand break at or near the one or more protospacer sequence of the 7 LPP, wherein the homology directed repair comprises insertion of the first donor polynucleotide or set of first PCR fragments at the double-strand break to provide a host cell with 7 copies of the first donor polynucleotide in its genome.

In one embodiment, during step c) the first temperature allows the one or more first guide RNA to form a complex at one or more protospacers of 8 LPP, and wherein the complex promotes the homology directed repair by creating a double-strand break at or near the one or more protospacer sequence of the 8 LPP, wherein the homology directed repair comprises insertion of the first donor polynucleotide or set of first PCR fragments at the double-strand break to provide a host cell with 8 copies of the first donor polynucleotide in its genome.

In one embodiment, during step c) the first temperature allows the one or more first guide RNA to form a complex at one or more protospacers of 9 LPP, and wherein the complex promotes the homology directed repair by creating a double-strand break at or near the one or more protospacer sequence of the 9 LPP, wherein the homology directed repair comprises insertion of the first donor polynucleotide or set of first PCR fragments at the double-strand break to provide a host cell with 9 copies of the first donor polynucleotide in its genome.

In one embodiment, during step c) the first temperature allows the one or more first guide RNA to form a complex at one or more protospacers of 10 LPP, and wherein the complex promotes the homology directed repair by creating a double-strand break at or near the one or more protospacer sequence of the 10 LPP, wherein the homology directed repair comprises insertion of the first donor polynucleotide or set of first PCR fragments at the double-strand break to provide a host cell with 10 copies of the first donor polynucleotide in its genome.

In one embodiment, during step c) the first temperature allows the one or more first guide RNA to form a complex at one or more protospacers of 11 LPP, and wherein the complex promotes the homology directed repair by creating a double-strand break at or near the one or more protospacer sequence of the 11 LPP, wherein the homology directed repair comprises insertion of the first donor polynucleotide or set of first PCR fragments at the double-strand break to provide a host cell with 11 copies of the first donor polynucleotide in its genome.

In one embodiment, during step c) the first temperature allows the one or more first guide RNA to form a complex at one or more protospacers of 12 LPP, and wherein the complex promotes the homology directed repair by creating a double-strand break at or near the one or more protospacer sequence of the 12 LPP, wherein the homology directed repair comprises insertion of the first donor polynucleotide or set of first PCR fragments at the double-strand break to provide a host cell with 12 copies of the first donor polynucleotide in its genome.

In one embodiment, during step c) the first temperature allows the one or more first guide RNA to form a complex at one or more protospacers of 13 LPP, and wherein the complex promotes the homology directed repair by creating a double-strand break at or near the one or more protospacer sequence of the 13 LPP, wherein the homology directed repair comprises insertion of the first donor polynucleotide or set of first PCR fragments at the double-strand break to provide a host cell with 13 copies of the first donor polynucleotide in its genome.

In one embodiment, during step c) the first temperature allows the one or more first guide RNA to form a complex at one or more protospacers of 14 LPP, and wherein the complex promotes the homology directed repair by creating a double-strand break at or near the one or more protospacer sequence of the 14 LPP, wherein the homology directed repair comprises insertion of the first donor polynucleotide or set of first PCR fragments at the double-strand break to provide a host cell with 14 copies of the first donor polynucleotide in its genome.

In one embodiment, during step c) the first temperature allows the one or more first guide RNA to form a complex at one or more protospacers of 15 LPP, and wherein the complex promotes the homology directed repair by creating a double-strand break at or near the one or more protospacer sequence of the 15 LPP, wherein the homology directed repair comprises insertion of the first donor polynucleotide or set of first PCR fragments at the double-strand break to provide a host cell with 15 copies of the first donor polynucleotide in its genome.

In one embodiment, during step c) the first temperature allows the one or more first guide RNA to form a complex at one or more protospacers of 16 LPP, and wherein the complex promotes the homology directed repair by creating a double-strand break at or near the one or more protospacer sequence of the 16 LPP, wherein the homology directed repair comprises insertion of the first donor polynucleotide or set of first PCR fragments at the double-strand break to provide a host cell with 16 copies of the first donor polynucleotide in its genome.

In one embodiment, during step b) there are delivered at least two first guide RNAs, each first guide RNA forming a complex with the nuclease at a first, second, third, fourth, or fifth protospacer.

In one embodiment, the at least two first guide RNAs comprise at least two of: a 1^{st} first guide RNA, a 2^{nd} first guide RNA, a 3^{rd} first guide RNA, a 4^{th} first guide RNA, and a 5^{th} first guide RNA.

In one embodiment, the 1^{st} first guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 2^{nd} first guide RNA, to the polynucleotide sequence of 3^{rd} first guide RNA, to the polynucleotide sequence of the 4^{th} first guide RNA, and to the polynucleotide sequence of the 5^{th} first guide RNA.

In one embodiment, the 2^{nd} first guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} first guide RNA, to the polynucleotide sequence of 3^{rd} first guide RNA, to the polynucleotide sequence of the 4^{th} first guide RNA, and to the polynucleotide sequence of the 5^{th} first guide RNA.

In one embodiment, the 3^{rd} first guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} first guide RNA, to the polynucleotide sequence of 2^{nd} first guide RNA, to the polynucleotide sequence of the 4^{th} first guide RNA, and to the polynucleotide sequence of the 5^{th} first guide RNA.

In one embodiment, the 4^{th} first guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} first guide RNA, to the polynucleotide sequence of 2^{nd} first guide RNA, to the polynucleotide sequence of the 3^{rd} first guide RNA, and to the polynucleotide sequence of the 5^{th} first guide RNA.

In one embodiment, the 5^{th} first guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} first guide RNA, to the polynucleotide sequence of 2^{nd} first guide RNA, to the polynucleotide sequence of the 3^{rd} first guide RNA, and to the polynucleotide sequence of the 4^{th} first guide RNA.

In one embodiment, the one or more first guide RNA comprises a 1^{st} first guide RNA, having a sequence complementary to a first protospacer sequence, a second protospacer sequence, a third protospacer sequence, a fourth protospacer sequence, or a fifth protospacer sequence, preferably the 1^{st} first guide RNA is complementary to a first protospacer sequence.

In one embodiment, the one or more first guide RNA comprises a 2^{nd} first guide RNA, having a sequence complementary to a first protospacer sequence, a second protospacer sequence, a third protospacer sequence, a fourth protospacer sequence, or a fifth protospacer sequence, preferably the 2^{nd} first guide RNA is complementary to a second protospacer sequence.

In one embodiment, the one or more first guide RNA comprises a 3^{rd} first guide RNA, having a sequence complementary to a first protospacer sequence, a second protospacer sequence, a third protospacer sequence, a fourth protospacer sequence, or a fifth protospacer sequence, preferably the 3^{rd} first guide RNA is complementary to a third protospacer sequence.

In one embodiment, the one or more first guide RNA comprises a 4^{th} first guide RNA, having a sequence complementary to a first protospacer sequence, a second protospacer sequence, a third protospacer sequence, a fourth protospacer sequence, or a fifth protospacer sequence, preferably the 4^{th} first guide RNA is complementary to a fourth protospacer sequence.

In one embodiment, the one or more first guide RNA comprises a 5^{th} first guide RNA, having a sequence complementary to a first protospacer sequence, a second protospacer sequence, a third protospacer sequence, a fourth protospacer sequence, or a fifth protospacer sequence, preferably the 5^{th} first guide RNA is complementary to a fifth protospacer sequence.

In one embodiment, the at least one guide RNA comprises a first guide RNA and a second guide RNA, and wherein the method additionally comprises step d):
d) cultivating the host cell at a second temperature allowing one or more second guide RNA of the at least one guide RNA to form a complex with the nuclease at one or more protospacer of the one or more LPP, and wherein the complex promotes the homology directed repair by creating a double-strand break at or near the one or more protospacer sequence of the one or more LPP, wherein the homology directed repair comprises insertion of the first donor polynucleotide and/or set of first PCR fragments at the double-strand break.

In one embodiment, during step b) there is additionally delivered one or more second polynucleotide comprising in 5′ to 3′ direction: a 5′ homology arm, a second polynucleotide encoding a second polypeptide of interest, and a 3′ homology arm, OR a set of one or more second PCR fragments comprising a second 3′ homology arm, a second 5′ homology arm, and a second polynucleotide encoding a second polypeptide of interest,
wherein the 3′ homology arm is identical to the 3′ homology arm of one or more LPP and wherein the 5′ homology arm is identical to the 5′ homology arm of one or more LPP, and
wherein during step c) the homology directed repair comprises insertion of the second donor polynucleotide or set of second PCR fragments at the double-strand break.

In another embodiment, during step b) there is additionally delivered one or more first polynucleotide comprising in 5′ to 3′ direction: a second 5′ homology arm, a first polynucleotide encoding the first polypeptide of interest, and a second 3′ homology arm, OR a set of one or more first PCR fragments comprising a second 3′ homology arm, a second 5′ homology arm, and the first polynucleotide encoding the first polypeptide of interest,
wherein the second 3′ homology arms and the second 5′ homology arms have less than 100% sequence identity to the 3′ and 5′ homology arms of the at least two LPP, respectively, e.g., less than 99%, less than 98%, less than 97%, less than 96%, less than 95%, less than 90%, less thank 85%, less than 80%, less than 75%, less than 70%, or less than 65%, and
wherein during step c) the homology directed repair comprises insertion of the first donor polynucleotide or set of PCR fragments at the double-strand break between a second 3′ homology arm and a second 5′ homology arm.

In one embodiment, the second guide RNA is complementary to one or more first protospacers, wherein during step d) the complex is formed at one or more first protospacer of the one or more LPP, creating a double-strand break at or near the one or more first protospacer.

In one embodiment, the second guide RNA is complementary to one or more second protospacers, wherein during step d) the complex is formed at one or more second protospacer of the one or more LPP, creating a double-strand break at or near the one or more second protospacer.

In one embodiment, the second guide RNA is complementary to one or more third protospacers, wherein during step d) the complex is formed at one or more third protospacer of the one or more LPP, creating a double-strand break at or near the one or more third protospacer.

In one embodiment, wherein the second guide RNA is complementary to one or more fourth protospacers, wherein during step d) the complex is formed at one or more fourth protospacer of the one or more LPP, creating a double-strand break at or near the one or more fourth protospacer.

In one embodiment, the second guide RNA is complementary to one or more fifth protospacers, wherein during step d) the complex is formed at one or more fifth protospacer of the one or more LPP, creating a double-strand break at or near the one or more fifth protospacer.

In one embodiment, during step d) the second temperature allows the one or more second guide RNA to form a complex at one or more protospacers of one LPP, and wherein the complex promotes the homology directed repair by creating a double-strand break at or near the one or more protospacer sequence of the one LPP, wherein the homology directed repair comprises insertion of the first donor polynucleotide and/or set of first PCR fragments at the double-strand break to provide a host cell with at least one copy of the first donor polynucleotide in its genome.

In one embodiment, during step d) the second temperature allows the one or more second guide RNA to form a complex at one or more protospacers of two LPP, and wherein the complex promotes the homology directed repair by creating a double-strand break at or near the one or more protospacer sequence of the two LPP, wherein the homology directed repair comprises insertion of the first donor polynucleotide and/or set of first PCR fragments at the double-strand break to provide a host cell with at least two copies of the first donor polynucleotide in its genome.

In one embodiment, during step d) the second temperature allows the one or more second guide RNA to form a complex at one or more protospacers of three LPP, and wherein the complex promotes the homology directed repair by creating a double-strand break at or near the one or more protospacer sequence of the three LPP, wherein the homology directed repair comprises insertion of the first donor polynucleotide and/or set of first PCR fragments at the double-strand break to provide a host cell with at least three copies of the first donor polynucleotide in its genome.

In one embodiment, during step d) the second temperature allows the one or more second guide RNA to form a complex at one or more protospacers of four LPP, and wherein the complex promotes the homology directed repair by creating a double-strand break at or near the one or more protospacer sequence of the four LPP, wherein the homology directed repair comprises insertion of the first donor polynucleotide and/or set of first PCR fragments at the double-strand break to provide a host cell with at least four copies of the first donor polynucleotide in its genome.

In one embodiment, during step d) the second temperature allows the one or more second guide RNA to form a complex at one or more protospacers of five LPP, and wherein the complex promotes the homology directed repair by creating a double-strand break at or near the one or more protospacer sequence of the five LPP, wherein the homology directed repair comprises insertion of the first donor polynucleotide and/or set of first PCR fragments at the double-strand break to provide a host cell with at least five copies of the first donor polynucleotide in its genome.

In one embodiment, during step d) the second temperature allows the one or more second guide RNA to form a complex at one or more protospacers of six LPP, and wherein the complex promotes the homology directed repair by creating a double-strand break at or near the one or more protospacer sequence of the six LPP, wherein the homology directed repair comprises insertion of the first donor polynucleotide and/or set of first PCR fragments at the double-strand break to provide a host cell with at least six copies of the first donor polynucleotide in its genome.

In one embodiment, during step d) the second temperature allows the one or more second guide RNA to form a complex at one or more protospacers of 8 LPP, and wherein the complex promotes the homology directed repair by creating a double-strand break at or near the one or more protospacer sequence of the 8 LPP, wherein the homology directed repair comprises insertion of the first donor polynucleotide and/or set of first PCR fragments at the double-strand break to provide a host cell with at least 8 copies of the first donor polynucleotide in its genome.

In one embodiment, during step d) the second temperature allows the one or more second guide RNA to form a complex at one or more protospacers of 16 LPP, and wherein the complex promotes the homology directed repair by creating a double-strand break at or near the one or more protospacer sequence of the 16 LPP, wherein the homology directed repair comprises insertion of the first donor polynucleotide and/or set of first PCR fragments at the double-strand break to provide a host cell with at least 16 copies of the first donor polynucleotide in its genome.

In one embodiment, during step b) there are delivered at least two second guide RNAs, each second guide RNA forming a complex with the nuclease at a first, second, third, fourth, or fifth protospacer.

In one embodiment, wherein the at least two second guide RNAs comprise at least two of: a 1^{st} second guide RNA, a 2^{nd} second guide RNA, a 3^{rd} second guide RNA, a 4^{th} second guide RNA, and a 5^{th} second guide RNA.

In one embodiment, the 1^{st} second guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 2^{nd} second guide RNA, to the polynucleotide sequence of 3^{rd} second guide RNA, to the polynucleotide sequence of the 4^{th} second guide RNA, and to the polynucleotide sequence of the 5^{th} second guide RNA.

In one embodiment, the 2^{nd} second guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} second guide RNA, to the polynucleotide sequence of 3^{rd} second guide RNA, to the polynucleotide sequence of the 4^{th} second guide RNA, and to the polynucleotide sequence of the 5^{th} second guide RNA.

In one embodiment, the 3^{rd} second guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} second guide RNA, to the polynucleotide sequence of 2^{nd} second guide RNA, to the polynucleotide sequence of the 4^{th} second guide RNA, and to the polynucleotide sequence of the 5^{th} second guide RNA.

In one embodiment, the 4^{th} second guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} second guide RNA, to the polynucleotide sequence of 2^{nd} second guide RNA, to the polynucleotide sequence of the 3^{rd} second guide RNA, and to the polynucleotide sequence of the 5^{th} second guide RNA.

In one embodiment, the 5^{th} second guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} second guide RNA, to the polynucleotide sequence of 2^{nd} second guide RNA, to the polynucleotide sequence of the 3^{rd} second guide RNA, and to the polynucleotide sequence of the 4^{th} second guide RNA.

In one embodiment, the one or more second guide RNA comprises a 1^{st} second guide RNA, having a sequence complementary to a first protospacer sequence, a second protospacer sequence, a third protospacer sequence, a fourth protospacer sequence, or a fifth protospacer sequence, preferably the 1^{st} second guide RNA is complementary to a first protospacer sequence.

In one embodiment, the one or more second guide RNA comprises a 2^{nd} second guide RNA, having a sequence complementary to a first protospacer sequence, a second protospacer sequence, a third protospacer sequence, a fourth protospacer sequence, or a fifth protospacer sequence, preferably the 2^{nd} second guide RNA is complementary to a second protospacer sequence.

In one embodiment, the one or more second guide RNA comprises a 3^{rd} second guide RNA, having a sequence complementary to a first protospacer sequence, a second protospacer sequence, a third protospacer sequence, a fourth protospacer sequence, or a fifth protospacer sequence, preferably the 3^{rd} second guide RNA is complementary to a third protospacer sequence.

In one embodiment, the one or more second guide RNA comprises a 4^{th} second guide RNA, having a sequence complementary to a first protospacer sequence, a second protospacer sequence, a third protospacer sequence, a fourth protospacer sequence, or a fifth protospacer sequence, preferably the 4^{th} second guide RNA is complementary to a fourth protospacer sequence.

In one embodiment, the one or more second guide RNA comprises a 5^{th} second guide RNA, having a sequence complementary to a first protospacer sequence, a second protospacer sequence, a third protospacer sequence, a fourth protospacer sequence, or a fifth protospacer sequence, preferably the 5^{th} second guide RNA is complementary to a fifth protospacer sequence.

In one embodiment, the at least one guide RNA comprises a first guide RNA, a second guide RNA, and a third guide RNA, and wherein the method additionally comprises the step
e) cultivating the host cell at a third temperature allowing one or more third guide RNA of the at least one guide RNA to form a complex with the nuclease at one or more protospacer of the one or more LPP, and
wherein the complex promotes the homology directed repair by creating a double-strand break at or near the one or more protospacer sequence of the one or more LPP, wherein the homology directed repair comprises insertion of the first donor polynucleotide and/or set of first PCR fragments at the double-strand break.

In one embodiment, the third guide RNA is complementary to one or more first protospacers, wherein during step e) the complex is formed at one or more first protospacer of the one or more LPP, creating a double-strand break at or near the one or more first protospacer.

In one embodiment, the third guide RNA is complementary to one or more second protospacers, wherein during step e) the complex is formed at one or more second protospacer of the one or more LPP, creating a double-strand break at or near the one or more second protospacer.

In one embodiment, the third guide RNA is complementary to one or more third protospacers, wherein during step e) the complex is formed at one or more third protospacer of the one or more LPP, creating a double-strand break at or near the one or more third protospacer.

In one embodiment, the third guide RNA is complementary to one or more fourth protospacers, wherein during step e) the complex is formed at one or more fourth protospacer of the one or more LPP, creating a double-strand break at or near the one or more fourth protospacer.

In one embodiment, the third guide RNA is complementary to one or more fifth protospacers, wherein during step e) the complex is formed at one or more fifth protospacer of the one or more LPP, creating a double-strand break at or near the one or more fifth protospacer.

In one embodiment, during step e) the third temperature allows the one or more third guide RNA to form a complex at one or more protospacers of one LPP, and wherein the complex promotes the homology directed repair by creating a double-strand break at or near the one or more protospacer sequence of the one LPP, wherein the homology directed repair comprises insertion of the first donor polynucleotide and/or set of first PCR fragments at the double-strand break to provide a host cell with at least one copy of the first donor polynucleotide in its genome.

In one embodiment, during step e) the third temperature allows the one or more third guide RNA to form a complex at one or more protospacers of two LPP, and wherein the complex promotes the homology directed repair by creating a double-strand break at or near the one or more protospacer sequence of the two LPP, wherein the homology directed repair comprises insertion of the first donor polynucleotide and/or set of first PCR fragments at the double-strand break to provide a host cell with at least two copies of the first donor polynucleotide in its genome.

In one embodiment, during step e) the third temperature allows the one or more third guide RNA to form a complex at one or more protospacers of three LPP, and wherein the complex promotes the homology directed repair by creating a double-strand break at or near the one or more protospacer sequence of the three LPP, wherein the homology directed repair comprises insertion of the first donor polynucleotide and/or set of first PCR fragments at the double-strand break to provide a host cell with at least three copies of the first donor polynucleotide in its genome.

In one embodiment, during step e) the third temperature allows the one or more third guide RNA to form a complex at one or more protospacers of four LPP, and wherein the complex promotes the homology directed repair by creating a double-strand break at or near the one or more protospacer sequence of the four LPP, wherein the homology directed repair comprises insertion of the first donor polynucleotide and/or set of first PCR fragments at the double-strand break to provide a host cell with at least four copies of the first donor polynucleotide in its genome.

In one embodiment, during step e) the third temperature allows the one or more third guide RNA to form a complex at one or more protospacers of five LPP, and wherein the complex promotes the homology directed repair by creating a double-strand break at or near the one or more protospacer sequence of the five LPP, wherein the homology directed repair comprises insertion of the first donor polynucleotide and/or set of first PCR fragments at the double-strand break to provide a host cell with at least five copies of the first donor polynucleotide in its genome.

In one embodiment, during step e) the third temperature allows the one or more third guide RNA to form a complex at one or more protospacers of six LPP, and wherein the complex promotes the homology directed repair by creating a double-strand break at or near the one or more protospacer sequence of the six LPP, wherein the homology directed repair comprises insertion of the first donor polynucleotide and/or set of first PCR fragments at the double-strand break to provide a host cell with at least six copies of the first donor polynucleotide in its genome.

In one embodiment, during step e) the third temperature allows the one or more third guide RNA to form a complex at one or more protospacers of 8 LPP, and wherein the complex promotes the homology directed repair by creating a double-strand break at or near the one or more protospacer sequence of the 8 LPP, wherein the homology directed repair comprises insertion of the first donor polynucleotide and/or set of first PCR fragments at the double-strand break to provide a host cell with at least 8 copies of the first donor polynucleotide in its genome.

In one embodiment, during step e) the third temperature allows the one or more third guide RNA to form a complex at one or more protospacers of 16 LPP, and wherein the complex promotes the homology directed repair by creating a double-strand break at or near the one or more protospacer sequence of the 16 LPP, wherein the homology directed repair comprises insertion of the first donor polynucleotide and/or set of first PCR fragments at the double-strand break to provide a host cell with at least 16 copies of the first donor polynucleotide in its genome.

In one embodiment, during step b) there are delivered at least two third guide RNAs, each third guide RNA forming a complex with the nuclease at a first, second, third, fourth, or fifth protospacer.

In one embodiment, the at least two third guide RNAs comprise at least two of: a 1^{st} third guide RNA, a 2^{nd} third guide RNA, a 3^{rd} third guide RNA, a 4^{th} third guide RNA, and a 5^{th} third guide RNA.

In one embodiment, the 1^{st} third guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 2^{nd} third guide RNA, to the polynucleotide sequence of 3^{rd} third guide RNA, to the polynucleotide sequence of the 4^{th} third guide RNA, and to the polynucleotide sequence of the 5^{th} third guide RNA.

In one embodiment, the 2^{nd} third guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} third guide RNA, to the polynucleotide sequence of 3^{rd} third guide RNA, to the polynucleotide sequence of the 4^{th} third guide RNA, and to the polynucleotide sequence of the 5^{th} third guide RNA.

In one embodiment, the 3^{rd} third guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} third guide RNA, to the polynucleotide sequence of 2^{nd} third guide RNA, to the polynucleotide sequence of the 4^{th} third guide RNA, and to the polynucleotide sequence of the 5^{th} third guide RNA.

In one embodiment, the 4^{th} third guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} third guide RNA, to the polynucleotide sequence of 2^{nd} third guide RNA, to the polynucleotide sequence of the 3^{rd} third guide RNA, and to the polynucleotide sequence of the 5^{th} third guide RNA.

In one embodiment, the 5^{th} third guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} third guide RNA, to the polynucleotide sequence of 2^{nd} third guide RNA, to the polynucleotide sequence of the 3^{rd} third guide RNA, and to the polynucleotide sequence of the 4^{th} third guide RNA.

In one embodiment, the one or more third guide RNA comprises a 1^{st} third guide RNA, having a sequence complementary to a first protospacer sequence, a second protospacer sequence, a third protospacer sequence, a fourth protospacer sequence, or a fifth protospacer sequence, preferably the 1^{st} third guide RNA is complementary to a first protospacer sequence.

In one embodiment, the one or more third guide RNA comprises a 2^{nd} third guide RNA, having a sequence complementary to a first protospacer sequence, a second protospacer sequence, a third protospacer sequence, a fourth protospacer sequence, or a fifth protospacer sequence, preferably the 2^{nd} third guide RNA is complementary to a second protospacer sequence.

In one embodiment, the one or more third guide RNA comprises a 3^{rd} third guide RNA, having a sequence complementary to a first protospacer sequence, a second protospacer sequence, a third protospacer sequence, a fourth protospacer sequence, or a fifth protospacer sequence, preferably the 3^{rd} third guide RNA is complementary to a third protospacer sequence.

In one embodiment, the one or more third guide RNA comprises a 4^{th} third guide RNA, having a sequence complementary to a first protospacer sequence, a second protospacer sequence, a third protospacer sequence, a fourth protospacer sequence, or a fifth protospacer sequence, preferably the 4^{th} third guide RNA is complementary to a fourth protospacer sequence.

In one embodiment, the one or more third guide RNA comprises a 5^{th} third guide RNA, having a sequence complementary to a first protospacer sequence, a second protospacer sequence, a third protospacer sequence, a fourth protospacer sequence, or a fifth protospacer sequence, preferably the 5^{th} third guide RNA is complementary to a fifth protospacer sequence.

In one embodiment, the at least one guide RNA comprises a first guide RNA, a second guide RNA, a third guide RNA, and a fourth guide RNA, and wherein the method additionally comprises the step f cultivating the host cell at a fourth temperature allowing one or more fourth guide RNA of the at least one guide RNA to form a complex with the nuclease at one or more protospacer of the one or more LPP, and wherein the complex promotes the homology directed repair by creating a double-strand break at or near the one or more protospacer sequence of the one or more LPP, wherein the homology directed repair comprises insertion of the first donor polynucleotide and/or set of first PCR fragments at the double-strand break.

In one embodiment, the at least one guide RNA comprises a first guide RNA, a second guide RNA, a third guide RNA, a fourth guide RNA, and a fifth guide RNA, and wherein the method additionally comprises the step
g) cultivating the host cell at a fifth temperature allowing one or more fifth guide RNA of the at least one guide RNA to form a complex with the nuclease at one or more protospacer of the one or more LPP, and wherein the complex promotes the homology directed repair by creating a double-strand break at or near the one or more protospacer sequence of the one or more LPP, wherein the homology directed repair comprises insertion of the first donor polynucleotide and/or set of first PCR fragments at the double-strand break.

In one embodiment, the first temperature is in the range of 15-50 C°, such as in the range of 22-42 C°, such as in the range of 23-39 C°, such as in the range of 25-37 C°.

In one embodiment, second temperature is in the range of 15-50 C°, such as in the range of 22-42 C°, such as in the range of 23-39 C°, such as in the range of 25-37 C°.

In one embodiment, the third temperature is in the range of 15-50 C°, such as in the range of 22-42 C°, such as in the range of 23-39 C°, such as in the range of 25-37 C°.

In one embodiment, the fourth temperature is in the range of 15-50 C°, such as in the range of 22-42 C°, such as in the range of 23-39 C°, such as in the range of 25-37 C°.

In one embodiment, the fifth temperature is in the range of 15-50 C°, such as in the range of 22-42 C°, such as in the range of 23-39 C°, such as in the range of 25-37 C°.

In one embodiment, the first temperature and the second temperature are in the range of 15-50 C°, such as in the range of 22-42 C°, such as in the range of 23-39 C°, such as in the range of 25-37 C°.

In one embodiment, the first temperature, the second temperature, and the third temperature are in the range of 15-50 C°, such as in the range of 22-42 C°, such as in the range of 23-39 C°, such as in the range of 25-37 C°.

In one embodiment, the first temperature, the second temperature, the third temperature, and the fourth temperature are in the range of 15-50 C°, such as in the range of 22-42 C°, such as in the range of 23-39 C°, such as in the range of 25-37 C°.

In one embodiment, the first temperature, the second temperature, the third temperature, the fourth temperature, and the fifth temperature are in the range of 15-50 C°, such as in the range of 22-42 C°, such as in the range of 23-39 C°, such as in the range of 25-37 C°.

In one embodiment, the first temperature differs from the second temperature by at least 0.5 C°, at least 1 C°, at least 1.5 C°, at least 2 C°, at least 2.5 C°, at least 3 C°, at least 3.5 C°, at least 4 C°, at least 4.5 C°, at least 5 C°, at least 5.5 C°, or at least 6 C°.

In one embodiment, the second temperature differs from the third temperature by at least 0.5 C°, at least 1 C°, at least 1.5 C°, at least 2 C°, at least 2.5 C°, at least 3 C°, at least 3.5 C°, at least 4 C°, at least 4.5 C°, at least 5 C°, at least 5.5 C°, or at least 6 C° In one embodiment, the third temperature differs from the fourth temperature by at least 0.5 C°, at least 1 C°, at least 1.5 C°, at least 2 C°, at least 2.5 C°, at least 3 C°, at least 3.5 C°, at least 4 C°, at least 4.5 C°, at least 5 C°, at least 5.5 C°, or at least 6 C° In one embodiment, the fourth temperature differs from the fifth temperature by at least 0.5 C°, at least 1 C°, at least 1.5 C°, at least 2 C°, at least 2.5 C°, at least 3 C°, at least 3.5 C°, at least 4 C°, at least 4.5 C°, at least 5 C°, at least 5.5 C°, or at least 6 C° In one embodiment, the first temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the second temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the third temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the fourth temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the fifth temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the first temperature and the second temperature are selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the first temperature, the second temperature, and the third temperature are selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the first temperature, the second temperature, the third temperature, and the fourth temperature are selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the first temperature, the second temperature, the third temperature, the fourth temperature, and the fifth temperature are selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.

In one embodiment, the first temperature is in the range of 22-40 C°, and the second temperature is in the range of 35-50 C°.

In one embodiment, the first donor polynucleotide does not comprise a selectable marker or counter-selectable marker.

In one embodiment, the first donor polynucleotide comprises a selectable or counter-selectable marker.

In one embodiment, during step b) there is furthermore delivered a second donor polynucleotide comprising in 5′ to 3′ direction: a 5′ homology arm, a second polynucleotide encoding a second polypeptide of interest, and a 3′ homology arm, wherein the 3′ homology arm is identical to the 3′ homology arm of the host cell, and wherein the 5′ homology arm is identical to the 5′ homology arm of the host cell.

In one embodiment, the first polypeptide of interest comprises a brazzein, a casein, a patatin, an ovalbumin, an osteopontin, an ovotransferrin, an ovomucin, an ovomucoid, an ovostatin, a glycomacropeptide, a lactoferrin, an alpha-lactalbumin, e.g., bovine alpha-lactalbumin, a beta-lactalbumin and/or a collagen.

In one embodiment, the first polypeptide of interest comprises a therapeutic polypeptide selected from the group consisting of an antibody, an antibody fragment, an antibody-based drug, a Fc fusion protein, an anticoagulant, a blood factor, a bone morphogenetic protein, an engineered protein scaffold, an enzyme, a growth factor, a blood clotting factor, a hormone, an interferon (such as an interferon alpha-2b), an interleukin, a lactoferrin, an alpha-lactalbumin, a beta-lactalbumin, an ovomucoid, an ovostatin, a cytokine, an obestatin, a human galactosidase (such as an human alpha-galactosidase A), a vaccine, a protein vaccine, and a thrombolytic.

In one embodiment, first polypeptide of interest comprises an enzyme.

In one embodiment, the enzyme is selected from the list of a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

In one embodiment, the second polypeptide of interest comprises a polypeptide selected from the list of a transcription factor (e.g. amyR), a chaperone (e.g. BipA, PdiA, or PdiB), a heme pathway enzyme (e.g. encoded by hemA, or hemB), or a glycosylation pathway enzyme (e.g. a Mannosidase I, a GlcNAc transferase, a galactosyltransferase, a sialyltransferase, an oligosaccharyltransferase, or an glucosidase).

In one embodiment, the method additionally comprises step h), subsequent of any of steps c), d), e), f or g):
providing a host cell comprising a first polynucleotide in one or more LPP, said host cell being generated with the method of any of the previous paragraphs, and said host cell comprising at least one LPP not comprising the first polynucleotide (=empty LPP),
delivering to the host cell a RNA-guided DNA nuclease, and at least one guide RNA directed to one or more protospacer of one or more empty LPP, and
cultivating the host cell at a temperature allowing the at least one guide RNA to form a complex with the nuclease at the one or more empty LPP, wherein the complex promotes the homology directed repair by creating a double-strand break at or near the one or more protospacer sequence of the one or more empty LPP, wherein the homology directed repair comprises insertion of the first polynucleotide at the double-strand break of the empty LPP.

### Methods of Production

In a third aspect, the present invention relates to a method of producing a polypeptide of interest, comprising cultivating a multi-copy host cell under conditions conducive for production of the polypeptide, said multi-copy host cell being generated by the method of the second aspect.

In one embodiment, the method further comprises recovering the polypeptide of interest.

The host cell is cultivated in a nutrient medium suitable for production of the polypeptide using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptide may be recovered from the medium using methods known in the art, including, but not limited to, collection, centrifugation, filtration, extraction, spray-drying, evaporation, or precipitation.

In one aspect, a whole fermentation broth comprising the polypeptide is recovered. In another aspect, a cell-free fermentation broth comprising the polypeptide is recovered.

The polypeptide may be purified by a variety of procedures known in the art to obtain substantially pure polypeptides and/or polypeptide fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the polypeptide is not recovered.

The present invention is further described by the following examples that should not be construed as limiting the scope of the invention.

### Examples

TABLE 1
Sequence listing overview

| SEQ ID NO: | sequence info |
|---|---|
| 1 | SYN2L (left flank) |
| 2 | SYN2R (right flank) |
| 3 | PS-59 protospacer |
| 4 | PS-73 protospacer |
| 5 | PS-76 protospacer |
| 6 | PS-11 protospacer |
| 7 | PS-93 protospacer |
| 8 | PS-94 protospacer |
| 9 | PS-75 protospacer |
| 10 | PS-49 protospacer |
| 11 | PS-89 protospacer |
| 12 | PS-74 protospacer |
| 13 | PS-48 protospacer |
| 14 | five protospacers PS-59, PS-73, PS-74, PS-75 and |
| | PS-76 for integration in the pepAb locus (=LPP#1) |
| 15 | five protospacers PS-59, PS-73, PS-74, PS-75 and |
| | PS-11 for integration in the pepE locus (=LPP#2) |
| 16 | five protospacers PS-59, PS-73, PS-48, PS-49 and |
| | PS-93 for integration in the amdS locus (=LPP#3) |
| 17 | five protospacers PS-59, PS-73, PS-48, PS-89 and |
| | PS-94 for integration in the pepAa locus (=LPP#4) |
| 18 | gRNA against PS-59 |
| 19 | gRNA against PS-73 |
| 20 | gRNA against PS-75 |
| 21 | gRNA against PS-94 |
| 22 | LPP#1 forward |
| 23 | LPP#1 reverse |
| 24 | LPP#2 forward |
| 25 | LPP#2 reverse |
| 26 | LPP#3 forward |
| 27 | LPP#3 reverse |
| 28 | LPP#4 forward |
| 29 | LPP#4 reverse |
| 30 | PS-90 protospacer |
| 31 | PS-79 protospacer |
| 32 | PS-80 protospacer |
| 33 | five protospacers PS-59, PS-73, PS-74, PS-79 and |
| | PS-80 for integration at the AO090026000695 locus |
| 34 | five protospacers PS-59, PS-73, PS-48, PS-89 and |
| | PS-90 for integration in the nprA locus |
| 35 | 750_1FOR primer |
| 36 | 750_2REV primer |
| 37 | 750_3FW primer |
| 38 | 750_4REV primer |
| 39 | 750_5FW primer |
| 40 | 750_6REV primer |

### Materials and Methods

Unless otherwise stated, DNA manipulation and transformation were performed using standard methods of molecular biology as described in Sambrook et al. (1989) Molecular cloning: A laboratory 20 manual, Cold Spring Harbor lab., Cold Spring Harbor, NY; Ausubel, F. M. et al. (eds.) “Current protocols in Molecular Biology”, John Wiley and Sons, 1995; Harwood, C. R., and Cut-ting, S. M. (eds.) “Molecular Biological Methods for Bacillus”. John Wiley and Sons, 1990.

Purchased material (E. coli and kits) E. coli HST08 (Clontech) is used for plasmid construction and amplification. Amplified plasmids are recovered with Qiagen Plasmid Midi or Mini Kit (Qiagen). Ligation is done with NEBuilder HiFi DNA Assembly Cloning Kit (NEB) according to the manufactory instructions. GFX™ PCR DNA and Gel Band Purification Kit (Merck) is used for the purification of PCR fragments and extraction of DNA fragment from agarose gel.

Amplified plasmids were recovered with Qiagen Plasmid Kit (Qiagen). DNA fragments were gel purified using the Qiagen MinElute Gel Extraction kit (Qiagen). Ligation reactions were carried out using the NEBUILDER@HiFi DNA Assembly Cloning Kit (New England Biolabs Inc.) according to the manufacturer's instructions. Polymerase Chain Reaction (PCR) was carried out with Phusion® DNA Polymerase (Thermo Fisher Scientific). Sanger DNA sequencing of plasmid DNA was performed by GeneWiz from Azenta Life Science (GENEWIZ Germany GmbH, Germany). Sequence analysis was performed with the SnapGene version 6.1.0 (GSL Biotech LLC, United States). PCR from genomic DNA was carried out using Thermo Scientific Phusion® DNA Polymerase (Thermo Scientific).

Enzymes for DNA manipulations (e.g. restriction endonucleases, ligases etc.) were obtained from New England Biolabs, Inc. and were used according to the manufacturer's instructions.

### DNA Fragments

All DNA fragments used in this study was made synthetically by Integrated DNA Technologies (IDT).

Transformation of E. coli

E. coli chemically competent host strain was incubated with DNA for 20 minutes on ice. The solution was then transferred to 42 degrees for 2 minutes and 20× Volume of SOC medium was added. The solution was then incubated at 37 degrees for 1 hour at 700 rpm before spreading on LB+Ampicillin plates.

Transformation of Aspergillus oryzae

Transformation of Aspergillus species can be achieved using the general methods for yeast transformation.

Aspergillus oryzae host strain was inoculated in 100 ml of Sucrose medium (supplemented with 10 mM uridine if strain is pyrG-) and incubated for 16 h at 30° C. at 200 rpm. Pellets were collected and washed with 0.6 M MgSO4, and resuspended in 10 ml 1.2 M MgSO4 containing a commercial beta-glucanase product (GLUCANEX™, Novozymes A/S, Bagsvord, Denmark) at a final concentration of 40 mg per ml. The suspension was incubated at 30° C. at 50 rpm until protoplasts were formed, and then washed twice with STC buffer. The protoplasts were resuspended to a final concentration of 1×10⁷ protoplasts/ml. Approximately 1 μg of guide plasmid and 1.5 ug of repair DNA was added to 100 μl of the protoplast suspension, mixed gently, and incubated 5 minutes at Room Temperature. 400 ul of PEG solution was added and the protoplast suspension was incubated for 20 minutes at Room Temperature. After the addition of 8 ml of 50° C. Sucrose Agar, the reaction was poured onto Sucrose Agar plates and the plates were incubated at 30° C. for 4 days.

### Media

COVE trace metals solution was composed of 0.04 g of NaB4O7·10H2O, 0.4 g of CuSO4·5H2O, 1.2 g of FeSO4·7H2O, 0.7 g of MnSO4·H2O, 0.8 g of Na2MoO2·2H2O, 10 g of ZnSO4·7H2O, and deionized water to 1 liter.

50×COVE salts solution was composed of 26 g of KCl, 26 g of MgSO4·7H2O, 76 g of KH2PO4, 50 ml of COVE trace metals solution, and deionized water to 1 liter.

Sucrose Medium was composed of 342.3 g of sucrose, 20 ml of 50×COVE salts solution, 10 ml of 1 M Urea, and deionized water to 1 liter.

Sucrose Agar were composed of 342.3 g of sucrose, 20 ml of 50×COVE salts solution, 10 ml of 1 M Urea, 20 g of granulated agar, and deionized water to 1 liter. If bar selection is used, Basta (Bayer A/S) is added to a final concentration of 0.11% glufosinat (w/V).

STC buffer was composed of 0.8 M sorbitol, 25 mM Tris pH 8, and 25 mM CaCl2.

PEG solution was composed of 60% PEG 4000 in STC buffer.

LB plus Ampicillin plates were composed of 10 g of tryptone, 5 g of yeast extract, 5 g of sodium chloride, 15 g of Bacto agar, ampicillin at 150 μg per ml, and deionized water to 1 liter.

SOC medium was composed of 20 g of tryptone, 5 g of yeast extract, 0.5 g of NaCl, 10 ml of 250 mM KCl, and deionized water to 1 liter.

COVE Urea Zeocin plates were composed of 342.3 g of sucrose, 20 ml of COVE salts solution, 10 ml 1 M urea, 25 g of Noble agar, and deionized water to 1 liter. The pH was adjusted to pH 6.5. After sterilisation, 1 mL 100 mg/mL Zeocin (Gibco™, Thermo Fisher Scientific).

PDA plates were composed of 39 g of potato dextrose agar (Difco) and deionized water to 1 liter.

PEG+G buffer was composed of 60% polyethylene glycol (PEG) 4000, 20% w/v glucose, 10 mM Tris-HCl pH 7.5, and 10 mM CaCl2 in deionized water. The solution is filter sterilized.

STC+G was composed of 1 M sorbitol, w/v glucose, 10 mM Tris pH 7.5, and 10 mM CaCl2 in deionized water.

STC was composed of 1 M sorbitol, 10 mM Tris pH 7.5, and 10 mM CaCl2 in deionized water.

YPD medium was composed of 1% yeast extract, 2% peptone, and 2% glucose in deionized water.

### Microbial Strains

Trichoderma reesei strain SAMF128-2A 11-1 is described in WO 20112911.

ddPCR (Digital Droplet PCR) for copy number determination

The ddPCR assays was run according to protocols available for the QX200 Bio-Rad ddPCR system. DNA was prepared by inoculating fungal spores in water added glass beads shaking vigorously for 2 minutes. An aliquot of DNA extract was transferred to the in-digest ddPCR solution. For digestion of DNA, one of the four restriction enzymes MseI, HindIII, AluI or HaeIII was used according to the suggestions by Bio-Rad for use in ddPCR assays. Probe assays (mix of fluorescent probe and PCR primers) were acquired from Integrated DNA Technologies (IDT). The assays were designed using the PrimerQuest™ Tool supplied by IDT. Droplets were made using the Bio-Rad QX200 AutoDG Droplet Digital PCR System. PCR was run using a Bio-Rad C1000 Touch Thermal Cycler with a 96-well module. Results were obtained using the QX200 droplet reader and results were analyzed using the QuataSoft™ version 1.7 software. All copy number results were obtained using an internal standard, which was either the wA gene or the prpA gene.

Generation of Trichoderma reesei protoplasts

Protoplast preparation and transformation of Trichoderma reesei were performed using a protocol similar to Penttila et al., 1987, Gene 61: 155-164. Briefly, T. reesei was cultivated in two shake flasks, each containing 25 mL of Y PD medium, at 27° C. for 17 hours with gentle agitation at 90 rpm. Mycelia were collected by filtration using a Vacuum Driven Disposable Filtration System (Millipore) and washed twice with deionized water and twice with 1.2 M sorbitol. Protoplasts were generated by suspending the washed mycelia in 30 mL of 1.2 M sorbitol containing 5 mg of YATALASE™ (Takara Bio USA, Inc.) per ml and 0.5 mg of chitinase (Sigma Chemical Co.) per ml for 60-75 minutes at 34° C. with gentle shaking at 90 rpm. Protoplasts were collected by centrifugation at 834×g for 7 minutes and washed twice with cold 1.2 M sorbitol. The protoplasts were counted using a hemocytometer and re-suspended to a final concentration of 1×10 8 protoplasts per ml of STC. Aliquots (1.1 ml) of the protoplast solution were placed in a Mr. FROSTY™ freezing container (Thermo Fisher Scientific) at −800 C for later use (as described in WO20123845).

### Trichoderma Transformation

Transformation of Trichoderma species can be achieved using the general methods for yeast transformation. The preferred procedure for this invention is described below. Approximately 1 μg of D27XZX or D278ZE plasmid DNA and 1 μg of plasmid D26V2Q DNA were combined (and added to 100 μl of the protoplast suspension of strain TT540 and then mixed gently. Then 250 pl PEG+G was added to the DNA-protoplast mixture, mixed gently and incubated at 34° C. for 30 minutes. One ml of STC+G was added, the protoplast suspension was mixed gently and poured onto Cove Urea Zeocin agar plates or PDA agar plates (with overlay with Hygromycin). The plates were incubated at 30° C. for 8-10 days.

Transformants were picked to PDA agar and incubated at 30° C. for 5-7 days. The strains were spore purified by diluting spores from the PDA agar plates in water and spreading onto Cove agar for a second round of selection (as described in WO20123845).

A portion of the D27XZX or D278ZE plasmid DNA was integrated into the genome by homologous recombination using the 5′ and 3′ flanks of the 70883 locus contained in the plasmids. The plasmid sequence between these 70883 homology flanks was integrated into the genome replacing the 70883 coding sequence. Transformants were selected for using the amdS selection marker contained between the 70883 flanking sequence within the plasmid. The resulting strains with the 70883 replacement and integrated intervening plasmid DNA sequence were named 0184PQ 5 (D27XZX in 016VA2), 01792Q (D278ZE in 016VA2), 0253QJ (D27XZX in 0154NN) and 016E5W (D278ZE in 0154NN).

### Example 1. Generation of Recipient SPADE Aspergillus Host Cell (Synthetic Protospacer Array Defined Entry)

A landing pad poynucleotide (LPP) is a DNA sequence consisting of three components, a protospacer array sequence consisting of one or more CRISPR target sites flanked by a left sequence and a right sequence. The two flanking sequences are used for integration of a repair construct by homologous recombination resulting in repair of the CRISPR guide facilitated chromosomal breaks in the landing pad.

For the design of the landing pads in this experiment, the left flank, named SYN2L (SEQ ID NO: 1), consists of 1000 base pairs (bp), i.e., having a length of 1000 nucleotides (nt). The SYN2L sequence was randomly generated by a python script setting the AT content to 52%. The right flank was named SYN2R (SEQ ID NO: 2), which is a 1000 bp sequence.

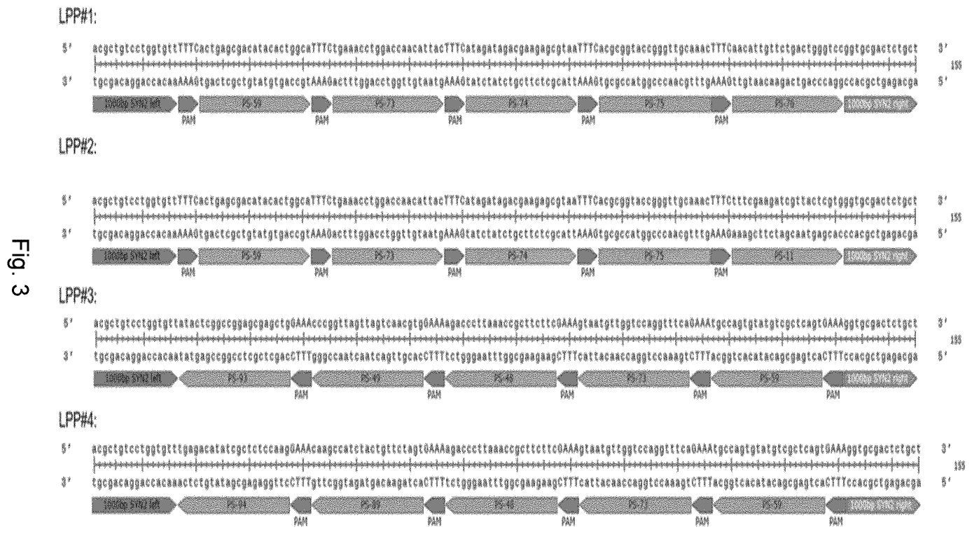

For this experiment, the protospacer array consists of five protospacer sequences. The protospacer sequences for the arrays were randomly generated by a python script setting the AT content to 52% on average. Moreover, the protospacers had all been tested to function at 30 degrees celsius using MAD7 as the CRISPR enzyme. As the 5′-PAM sequence, the sequence TTTC was chosen for all protospacers in the protospacer arrays. The location of the PAM sequence for each protospacer is shown in FIG. 3.

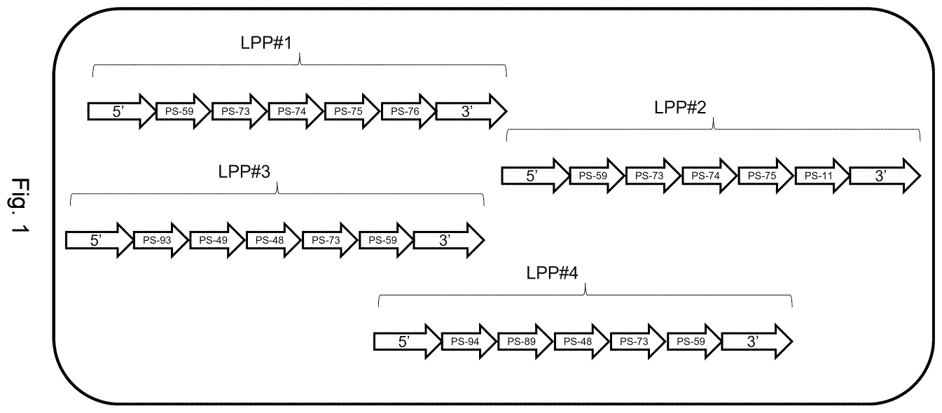

As shown in Table 2 and in FIG. 1, the four protospacer arrays of the four LPPs, all contain two first protospacer sequences #1A and #1B. One first protospacer sequence #1A is PS-59 (SEQ ID NO: 3) which is present in all four protospacer arrays, and another first protospacer sequence #1B is PS-73 (SEQ ID NO: 4) which is also present in all four protospacer arrays.

Moreover, each of the protospacer arrays contained three additional protospacers: a second protospacer (#2), a third protospacer (#3), and a fourth protospacer (#4). These protospacer sequences are either present in the protospacer arrays of three landing pads in total, two landing pads in total or are unique to the respective protospacer arrays. For an overview of the presence of protospacer sequences for each of the four landing pads LPP #1-LPP #4 see FIG. 1, and below Table 2.

TABLE 2

| | LPP#1 | LPP#2 | LPP#3 | LPP#4 |
|---|---|---|---|---|
| Protospacer | pepAb | pepE | amdS | pepAa |
| #1 A (specific | PS-59 (SEQ | PS-59 (SEQ | PS-59 (SEQ | PS-59 (SEQ |
| for all 4 LPP) | ID NO: 3) | ID NO: 3) | ID NO: 3) | ID NO: 3) |
| #1 B (specific | PS-73 (SEQ | PS-73 (SEQ | PS-73 (SEQ | PS-73 (SEQ |
| for all 4 LPP) | ID NO: 4) | ID NO: 4) | ID NO: 4) | ID NO: 4) |
| #2 (specific | PS-76 (SEQ | PS-11 (SEQ | PS-93 (SEQ | PS-94 (SEQ |
| for 1 LPP) | ID NO: 5) | ID NO: 6) | ID NO: 7) | ID NO: 8) |
| #3 (specific | PS-75 (SEQ | PS-75 (SEQ | PS-49 (SEQ | PS-89 (SEQ |
| for 1 or 2 LPP) | ID NO: 9) | ID NO: 9) | ID NO: 10) | ID NO: 11) |
| #4 (specific | PS-74 (SEQ | PS-74 (SEQ | PS-48 (SEQ | PS-48 (SEQ |
| for 2 LPP) | ID NO: 12) | ID NO: 12) | ID NO: 13) | ID NO: 13) |

For building the Aspergillus oryzae recipient strain, four chromosomal loci were chosen, which are present on four different chromosome arms to ensure genetic stability in the experiment. We chose the pepAb locus on chromosome 1, the pepE locus on chromosome 2, the amdS locus on chromosome 6 and the pepAa locus on chromosome 8.

The four LPPs were inserted at the four chromosomal loci using four CRISPR guide RNAs targeting each of the four loci as well as four repair fragments, which contained the four LPPs flanked by loci specific repair sequences. While integrating the LPPs, the loci specific genes were deleted thereby removing the guide targeting sequence.

The resulting Aspergillus oryzae recipient strain AT5885 containing the four LPPs at the four different chromosomal loci was confirmed by genome sequencing. The recipient strain AT5885 is ApyrG and therefore requires supplementation of uridine to be able to grow.

The full sequences of the four protospacer arrays have been listed as:
SEQ ID NO: 14 (LPP #1): the five protospacers PS-59, PS-73, PS-74, PS-75 and PS-76 for integration in the pepAb locus;
SEQ ID NO: 15 (LPP #2): the five protospacers PS-59, PS-73, PS-74, PS-75 and PS-11 for integration in the pepE locus;
SEQ ID NO: 16 (LPP #3): the five protospacers PS-59, PS-73, PS-48, PS-49 and PS-93 for integration in the amdS locus
SEQ ID NO: 17 (LPP #4): the five protospacers PS-59, PS-73, PS-48, PS-89 and PS-94 for integration in the pepAa locus.

### Example 2. One-Step Integration of 4 Gene Copies into 4 Landing Pad Polynucleotides (LPP) Using One gRNA Sequence

The recipient strain AT5885 comprising 4 LPPs was made transformable by protoplasting mycelia.

Two CRISPR guide RNA (gRNA) plasmids were built (pAT4859 and pAT4873), each containing a gRNA which enables cutting of all four LPPs in the recipient strain. pAT4859 comprises a first gRNA (SEQ ID NO:18) enabling cutting at the protospacer sequence PS-59, and pAT4873 comprises a first gRNA (SEQ ID NO: 19) enabling cutting at the protospacer sequence PS-73. Both guide RNA plasmids carry the pyrG gene for maintenance in the recipient strain upon transformation. An overview of designed gRNAs is shown in Table 3.

A repair DNA construct (=first donor polynucleotide), REPAIR1, was built, which contained a sequence of interest flanked by the repair flanks SYN2L (SEQ ID NO: 1) and SYN2R (SEQ ID NO: 2).

TABLE 3

| SEQ ID | gRNA | |
|---|---|---|
| NO: | target | gRNA sequence |
| 18 | PS-59 | GGAAUUUCUACUCUUGUAGAUacugagcgac |
| | | auacacuggca |
| 19 | PS-73 | GGAAUUUCUACUCUUGUAGAUacgcgguacc |
| | | ggguugcaaac |
| 20 | PS-75 | GGAAUUUCUACUCUUGUAGAUacgcgguacc |
| | | ggguugcaaac |
| 21 | PS-94 | GGAAUUUCUACUCUUGUAGAUcuuggagagc |
| | | gauaugucuca |

In Table 3 (right column), upper case letters in the guide RNA represent the tracrRNA (trans-activating CRISPR RNA), whereas lower case letters represent the crRNA (CRISPR RNA), which recognizes the protospacer in the target DNA.

Protoplasts of AT5885 were transformed with pAT4859 and repair DNA and transformants were selected on minimal plates without addition of uridine. The transformation efficiency was 100 transformants/ug of repair DNA. 20 transformants were spore reisolated twice and the copy number was determined by ddPCR. 75% of the strains had the expected four copies of the gene of interest, i.e. one copy per LPP. Moreover, ddPCR was used to verify that the protospacer arrays in the four LPPs were no longer present in those 75% correct transformants, which indicates that the repair and insertion was successful.

Similar results were obtained using the guide plasmid pAT4873 and the same repair DNA construct. Thus, we conclude that the SPADE technology can be used for efficient and controlled 1-step integration of multiple copies into multiple landing pads.

### Example 3. One-Step Integration of 3 Gene Copies into 3 LPPs, Using 2 Different gRNA Sequences

AT5885, comprising 4 LPPs, was made pyrG+ resulting in strain AT6412.

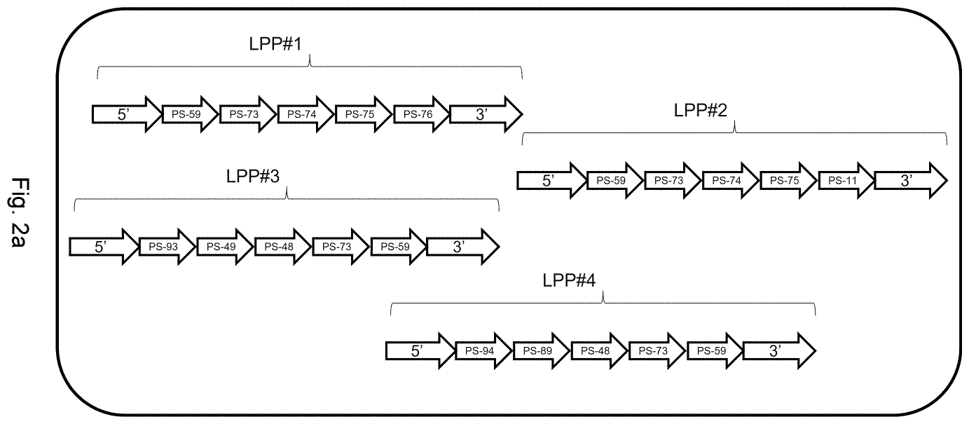

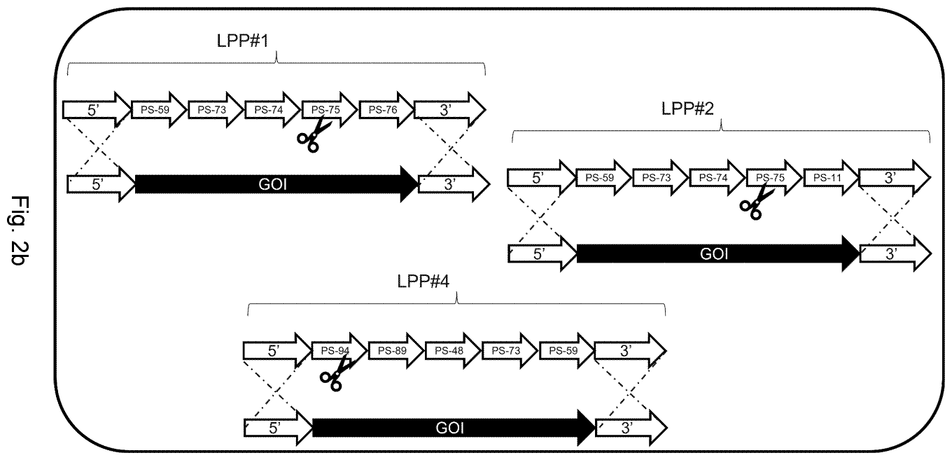

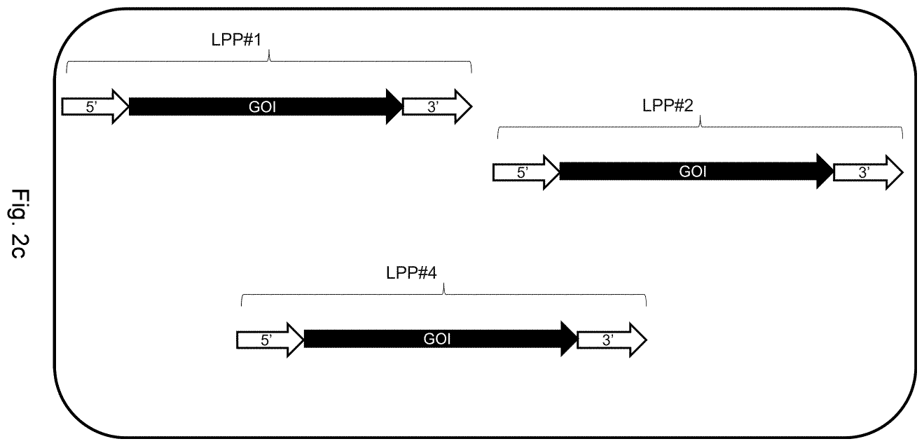

One CRISPR guide RNA plasmid was built (pAT6716), which enables cutting of three of the four LPPs in the recipient strain. pAT6716 comprises gRNA sequences (SEQ ID NO: 20) that allow DNA cutting at the protospacer sequence PS-75 (present in LPP #1 and LPP #2), and guide RNA (SEQ ID NO: 21) enabling cutting at the protospacer sequence PS-94 (present in LPP #4). The cutting of the three LPPs is indicated in FIG. 2a. The guide RNA plasmid carries the bar gene for maintenance in the recipient strain upon transformation on BASTA plates.

A repair DNA construct (=first donor polynucleotide), REPAIR1, was built. REPAIR1 contained a sequence of interest/gene of interest (GOI) flanked by the repair flanks (homology arms) SYN2L (SEQ ID NO: 1) and SYN2R (SEQ ID NO: 2). The GOI was subject to integration via homology directed repair at the sites were double strand breaks were introduced, see FIG. 2b.

Protoplasts of AT6412 were transformed with pAT6716 and repair DNA construct REPAIR1. Transformants were selected on minimal plates without addition of uridine. The transformation efficiency was 100+ transformants/ug of repair DNA. 20 transformants were spore reisolated twice and the copy number was determined by ddPCR. 80% of the strains had the expected three copies of the gene of interest, i.e., the sequence of interest was present in LPP #1, LPP #2, and LPP #4 (see FIG. 2c). Moreover, ddPCR was used to verify that the protospacer arrays in the three LPPs were no longer present in those 80% correct transformants, which indicates that the repair and insertion was successful. Moreover, it was verified that the fourth landing pad (LPP #3) was intact. Thus, the SPADE technology proves to be efficient for targeted integration into multiple landing pads using different guide RNA sequences. The shown one-step multiplexing approach allows controlled dosing of copy numbers during strain construction.

### Example 4. Multi-Step Integration of Gene Copies into Multiple LPPs

Example 4 is an example for “step h)” of the method according to the second aspect of the invention.

The AT6412 host strain comprising 4 LPPs (generated in Example 1) was used for this experiment.

A DNA repair fragment, REPAIR2 (=first donor polynucleotide), was designed containing an expression cassette encoding a protein of interest to which Aspergillus oryzae reacts negative/sensible, e.g. the host cells show reduced viabilities after transformation. Thus, the method described in example 2 is not preferred for making multi-copy strains for such proteins of interest.

A guide plasmid, pAT6654, comprising a gRNA sequence (SEQ ID NO: 20) enabling cutting of PS-75 present in two (LPP #1 and LPP #2) of the four LPPs in AT6412 was made.

A guide plasmid, pAT6657, comprising a gRNA sequence (SEQ ID NO: 21) enabling cutting of PS-94 present in one (LPP #4) of the four LPPs in AT6412 was made.

A guide plasmid, pAT6327, comprising a gRNA sequence (SEQ ID NO: 18) enabling cutting of PS-59 present in all four of the landing pads in AT6412 was made.

In step 1, the guide plasmid pAT6654 was transformed in AT6412 together with small amounts of REPAIR2 DNA, so the cells could survive the exposure. Transformants were spore isolated and strains having two copies of REPAIR2 integrated, one copy into LPP #1 and one copy into LPP #2, were characterized using ddPCR to determine the copy number and the integrity of the two other landing pads were determined as well using ddPCR. The final 2-copy strain was named AT6712.

The subsequent steps 2a and 2b were carried out in separate experiments, resulting in a 3-copy strain and in a 4-copy strain, respectively.

In step 2a, the guide plasmid pAT6557, cutting one of two remaining “empty” landing pads (LPP #4), was transformed in AT6712. Transformants were spore isolated and strains having three copies of REPAIR2 integrated were characterized by ddPCR. Correct transformants tested had the REPAIR2 fragment copied from one of the already occupied loci into the third locus LPP #4, resulting in a 3-copy strain.

In step 2b, the guide plasmid pAT6327, cutting both of the two remaining “empty” landing pads (LPP #3 and LPP #4), was transformed in AT6712. Transformants were spore isolated and strains having four copies of REPAIR2 integrated were characterized by ddPCR. Correct transformants tested had the REPAIR2 fragment copied from the already occupied loci to both loci LPP #3 and LPP #4, resulting in a 4-copy strain.

Normally, for generation of multicopy strains in one step, the host cells are transformed with high amount of DNA to increase integration efficiency and to secure integration into all desired landing pads. This high DNA load can be toxic for some genes of interest and/or for some host cells. Using the SPADE technology of example 4, multi-copy strains can also be generated for genes of interest which are toxic for the cells, as the presented two-step approach can keep the exposure to the toxic DNA at a minimum.

### Example 5. Possible Arrangements for Protospacer Presence in Up to 16 Landing Pads

Table 4 shows exemplary arrangements for protospacers in up to 16 landing pads.

Example 1 of Table 4 shows a host cell with 16 landing pads (LPP #1-LPP #16). Each LPP comprises between 1-5 protospacer sequences. Protospacer #1 is present in only one LPP. Protospacer #2 is present in two LPPs. Protospacer #3 is present in 4 LPPs. Protospacer #4 is present in 8 LPPs, and protospacer #5 is present in all 16 LPPs. Depending on how many copies of the gene of interest shall be incorporated into the host cell, a suitable protospacer can be targeted.

Example 2 of Table 4 shows a host cell with 16 landing pads (LPP #1-LPP #16). Each LPP comprises between 1-5 protospacer sequences. Protospacer #1 is present in only one LPP. Protospacer #2 is present in 4 LPPs. Protospacer #3 is present in 6 LPPs. Protospacer #4 is present in 10 LPPs, and protospacer #5 is present in all 16 LPPs. Depending on how many copies of the gene of interest shall be incorporated into the host cell, a suitable protospacer can be targeted.

These examples clearly show how versatile the LPPs can be designed to provide a flexible SPADE host cell which can be used for multi-copy integration, either in a one step approach, or in a multi-step approach.

TABLE 4

| | Protospacer | | | | | | | | |
|---|---|---|---|---|---|---|---|---|---|
| | # | LPP#1 | LPP#2 | LPP#3 | LPP#4 | LPP#5 | LPP#6 | LPP#7 | LPP#8 |
| Example | 1 | x | | | | | | | |
| 1 | 2 | x | x | | | | | | |
| | 3 | x | x | x | x | | | | |
| | 4 | x | x | x | x | x | x | x | x |
| | 5 | x | x | x | x | x | x | x | x |
| Exampl [symbol] | 1 | x | | | | | | | |
| | 2 | x | x | x | x | | | | |
| | 3 | x | x | x | x | x | x | | |
| | 4 | x | x | x | x | x | x | x | x |
| | 5 | x | x | x | x | x | x | x | x |
| | Protospacer | | | | | | | | |
| | # | LPP#9 | LPP#10 | LPP#11 | LPP#12 | LPP#13 | LPP#14 | LPP#15 | LPP#16 |
| Example | 1 | | | | | | | | |
| 1 | 2 | | | | | | | | |
| | 3 | | | | | | | | |
| | 4 | | | | | | | | |
| | 5 | x | x | x | x | x | x | x | x |
| Exampl [symbol] | 1 | | | | | | | | |
| | 2 | | | | | | | | |
| | 3 | | | | | | | | |
| | 4 | x | x | | | | | | |
| | 5 | x | x | x | x | x | x | x | x |

[symbol]

indicates data missing or illegible when filed

### Example 6. Generation of Recipient SPADE Trichoderma Host Cell

A landing pad polynucleotide (LPP) is a DNA sequence consisting of three components, a protospacer array sequence consisting of one or more CRISPR target sites flanked by a left sequence and a right sequence. The two flanking sequences are used for integration of a repair construct by homologous recombination resulting in repair of the CRISPR guide facilitated chromosomal breaks in the landing pad.

For the design of the landing pads in this experiment, the left flank, named SYN2L (SEQ ID NO: 1), consists of 1000 base pairs (bp), i.e., having a length of 1000 nucleotides (nt). The SYN2L sequence was randomly generated by a python script setting the AT content to 52%. The right flank was named SYN2R (SEQ ID NO: 2), which is a 1000 bp sequence.

For this experiment, the protospacer array consists of five protospacer sequences. The protospacer sequences for the arrays were randomly generated by a python script setting the AT content to 52% on average. Moreover, the protospacers had all been tested to function at 30 degrees Celsius using MAD7 as the CRISPR enzyme. As the 5′-PAM sequence, the sequence TTTC was chosen for all protospacers in the protospacer arrays. The location of the PAM sequence for each protospacer is shown in FIG. 3.

As shown in Table 2 and in FIG. 1 for the Aspergillus cells, the four protospacer arrays of the four LPPs in Trichoderma cells all contain two first protospacer sequences #1A and #1B. One first protospacer sequence #1A is PS-59 (SEQ ID NO: 3) which is present in all four protospacer arrays, and another first protospacer sequence #1B is PS-73 (SEQ ID NO: 4) which is also present in all four protospacer arrays.

Moreover, each of the protospacer arrays contained three additional protospacers: a second protospacer (#2), a third protospacer (#3), and a fourth protospacer (#4). These protospacer sequences are either present in the protospacer arrays of three landing pads in total, two landing pads in total or are unique to the respective protospacer arrays. For an overview of the presence of protospacer sequences for each of the four landing pads LPP #1-LPP #4 see Table 5.

TABLE 5

| | LPP#T1 | LPP#T2 | LPP#T3 | LPP#T4 |
|---|---|---|---|---|
| Protospacer | eg1 | tf37062 | tf92949 | xyn2 |
| #1 A (specific | PS-59 (SEQ | PS-59 (SEQ | PS-59 (SEQ | PS-59 (SEQ |
| for all 4 LPP) | ID NO: 3) | ID NO: 3) | ID NO: 3) | ID NO: 3) |
| #1 B (specific | PS-73 (SEQ | PS-73 (SEQ | PS-73 (SEQ | PS-73 (SEQ |
| for all 4 LPP) | ID NO: 4) | ID NO: 4) | ID NO: 4) | ID NO: 4) |
| #2 (specific | PS-89 (SEQ | PS-89 (SEQ | PS-74 (SEQ | PS-74 (SEQ |
| for 2 LPP) | ID NO: 11) | ID NO: 11) | ID NO: 12) | ID NO: 12) |
| #3 (specific | PS-90 (SEQ | PS-94 (SEQ | PS-75 (SEQ | PS-79 (SEQ |
| for 1 LPP) | ID NO: 30) | ID NO: 8) | ID NO: 9) | ID NO: 31) |
| #4 (specific | PS-48 (SEQ | PS-48 (SEQ | PS-76 (SEQ | PS-80 (SEQ |
| for 1 or 2 LPP) | ID NO: 13) | ID NO: 13) | ID NO: 5) | ID NO: 32) |

For building the Trichoderma reesei recipient strains, four chromosomal loci were chosen, which are present on four different chromosome arms to ensure genetic stability in the experiment. We chose the eg1 locus on chromosome 2, the tf37062 locus on chromosome 4, the tf92949 locus on chromosome 3 and the xyn2 locus on chromosome 2.

The four LPPs were inserted at the four chromosomal loci using four CRISPR guide RNAs targeting each of the four loci as well as four repair fragments, which contained the four LPPs flanked by loci specific repair sequences. While integrating the LPPs, the loci specific genes were deleted thereby removing the guide targeting sequence.

The resulting Trichoderma reesei recipient strain TT540 containing the four LPPs at the four different chromosomal loci was confirmed by digital droplet PCR.

The four Trichoderma protospacer arrays have been listed as:
LPP #T1: the five protospacers PS-59, PS-73, PS-89, PS-90 and PS-48 for integration in the eg1 locus;
LPP #T2: the five protospacers PS-59, PS-73, PS-89, PS-94 and PS-48 for integration in the tf37062 locus;
LPP #T3: the five protospacers PS-59, PS-73, PS-74, PS-75 and PS-76 for integration in the tf92949 locus
LPP #T4: the five protospacers PS-59, PS-73, PS-74, PS-79 and PS-80 for integration in the xyn2 locus.

Next to host TT540 with four LPP's, we also constructed a host with only one LLP, i.e. TT499 having the single LLP in the xyn2 locus.

### Example 7. One-Step Integration of 4 Gene Copies into 4 Landing Pad Polynucleotides (LPP) Using One gRNA Sequence

The recipient Trichoderma reesei strain TT540 comprising 4 LPPs was made transformable by protoplasting mycelia. One CRISPR guide RNA (gRNA) plasmids was built (pTT397) containing a gRNA which enables cutting of all four LPPs in the recipient strain. The plasmid comprised the first gRNA (SEQ ID NO:18) enabling cutting at the protospacer sequence PS-59. Plasmid pTT397 carries the hygromycin marker-gene for maintenance in the recipient strain upon transformation. An overview of designed gRNAs is shown in Table 6.

A repair DNA construct (=first donor polynucleotide), REPAIR3, was built, which contained a sequence of interest flanked by the repair flanks SYN2L (SEQ ID NO: 1) and SYN2R (SEQ ID NO: 2).

TABLE 6

| SEQ ID | gRNA | |
|---|---|---|
| NO: | target | gRNA sequence |
| 18 | PS-59 | GGAAUUUCUACUCUUGUAGAUacugagcga |
| | | cauacacuggca |
| 19 | PS-73 | GGAAUUUCUACUCUUGUAGAUacgcgguac |
| | | cggguugcaaac |

In Table 6 (right column), upper case letters in the guide RNA represent the tracrRNA (trans-activating CRISPR RNA), whereas lower case letters represent the crRNA (CRISPR RNA), which recognizes the protospacer in the target DNA.

Protoplasts of TT540 were transformed with pTT397 and repair DNA (Ascl-digested pTT391; EcoL1028-lysozyme; WO2021202479-A1) and transformants were selected on PDA plates with hygromycin addition. Ten transformants were spore reisolated twice and the copy number was determined by ddPCR. 75% of the transformants had the expected four copies of the gene of interest, i.e. one copy per LPP.

Similar results were obtained using the guide plasmid pTT397 and another repair DNA construct (Ascl-digested pTT508; Af BX beta-xylosidase; WO2022127892-A1). Thus, we conclude that the SPADE technology can be used for efficient and controlled 1-step integration of multiple copies into multiple landing pads in T. reesei.

### Example 8. One-Step Multiplex Integration of Two Different Genes into 4 LPPs, Using One gRNA Sequence

In this example, we transformed Trichoderma reesei recipient strain TT540 with guide plasmid pTT397 and applied two repair DNA constructs (=first donor polynucleotide), REPAIR4 and REPAIR5. REPAIR4 contained a first sequence of interest/gene of interest (GOI), i.e. pTT391: EcoL1028 lysozyme (WO2021202479-A1), flanked by the homology arms SYN2L (SEQ ID NO: 1) and SYN2R (SEQ ID NO: 2). REPAIR5 contained a second sequence of interest/gene of interest (GOI), i.e. pTT508: AfBX beta-xylosidase (WO2022127892-A1), flanked by the homology arms SYN2L (SEQ ID NO: 1) and SYN2R (SEQ ID NO: 2. 5-10 transformants were spore reisolated twice and the copy number for each gene of interest was determined by ddPCR. In all transformants the four LLPs were occupied with the genes of interest. We obtained varying copy number ratios of lysozyme versus beta-xylosidase, containing the combinations: zero to four (0:4), one to three (1:3), and three to one (3:1).

Thus, the SPADE technology proves to be efficient for targeted integration into multiple landing pads using different GOI sequences. The shown one-step multiplexing approach allows easy combination of GOIs in strain construction.

### Example 9. Efficiency of Applied Flank Length for One-Step Integration of One Gene Copy into 1 LPPs Using One gRNA Sequence

The Trichoderma reesei recipient strain TT499 comprising 1 LPP was made transformable by protoplasting mycelia. One CRISPR guide RNA (gRNA) plasmids was built (pTT505) containing a gRNA which enables cutting the LPP in the recipient strain. The plasmid comprised the first gRNA (SEQ ID NO:18) enabling cutting at the protospacer sequence PS-59. Plasmid pTT505 carries the zeocin marker-gene for maintenance in the recipient strain upon transformation. An overview of designed gRNAs is shown in Table 6.

In this example, 7 repair DNA constructs (=first donor polynucleotide), REPAIR6-12, were built, which each contained a sequence of interest flanked by the repair flanks SYN2L (SEQ ID NO: 1) and SYN2R (SEQ ID NO: 2). The repair flanks (homology arms) are varying in length from 1000, 750, 500, 250, 200, 150, to 100-bp, being shortened at the outside.

The gene of interest is EcoL1028 lysozyme (WO2021202479-A1). Protoplasts of TT499 were transformed with pTT505 and the seven different repair DNAs and transformants were selected on Cove plates supplemented with urea and zeocin. For each transformation applying the various repair constructs, we obtained transformants. The transformants obtained from each transformation were spore reisolated twice and the copy number was determined by ddPCR. All transformants had the expected single copy of the gene of interest. This example shows that SPADE integration can be performed using minimal 100-bp flanks.

### Example 10. One Step Generation of a 4-Copy Lipase Strain Using a Single gRNA Sequence

For building the Aspergillus oryzae recipient strain, four chromosomal loci were chosen, which are present on four different chromosome arms to ensure genetic stability in the experiment. We chose the pepAb locus on chromosome 1, the pepE locus on chromosome 2, the amdS locus on chromosome 6 and the pepAa locus on chromosome 8.

The four LPPs were inserted at the four chromosomal loci using four CRISPR guide RNAs targeting each of the four loci as well as four repair fragments, which contained the four LPPs flanked by loci specific repair sequences. While integrating the LPPs, the loci specific genes were deleted thereby removing the guide targeting sequence.

The resulting Aspergillus oryzae recipient strain A2F0012 containing the four LPPs at the four different chromosomal loci was confirmed by genome sequencing. The recipient strain A2F0012 is ApyrG and therefore requires supplementation of uridine to be able to grow.

The full sequences of the four protospacer arrays have been listed as:
SEQ ID NO: 14 (LPP #1): the five protospacers PS-59, PS-73, PS-74, PS-75 and PS-76 for integration in the pepAb locus;
SEQ ID NO: 15 (LPP #2): the five protospacers PS-59, PS-73, PS-74, PS-75 and PS-11 for integration in the pepE locus;
SEQ ID NO: 16 (LPP #3): the five protospacers PS-59, PS-73, PS-48, PS-49 and PS-93 for integration in the amdS locus
SEQ ID NO: 17 (LPP #4): the five protospacers PS-59, PS-73, PS-48, PS-89 and PS-94 for integration in the pepAa locus. DNA fragments for transformation

All DNA fragments used in this study were PCR amplified from gDNA and confirmed by sanger sequenced. The repair DNA construct (=first donor polynucleotide), REPAIR13, was built containing a sequence of interest flanked by the repair flanks SYN2L (SEQ ID NO: 1) and SYN2R (SEQ ID NO: 2).

Transformation of Aspergillus oryzae

Protoplast of the strain A2F0012 (pyrG-) containing LPP #1, #2, #3 and #4 was transformed by using CRISPR guide RNA (gRNA) pAT4859 containing a gRNA which enables cutting of all four LPPs in the recipient strain. pAT4859 comprises a gRNA (SEQ ID NO:18) enabling cutting at the protospacer sequence PS-59 present on all four LPPs, the guide RNA plasmids carry the pyrG gene for maintenance in the recipient strain upon transformation.

The Protoplasts of A2F0012 were transformed with pAT4859 and the 3 repair DNA fragments, the transformation was done on an automated platform using a Hamilton robots, the transformants were plated on sucrose plates without addition of uridine, and grown 4 days at 30° C., the transformation efficiency was 100 transformants; 5 transformants were selected, picked and transferred to sucrose agar in with urea and uridine MTP, the plate was incubated 3 days at 30° C., the copy number was determined by ddPCR giving 80% of transformants with 4 copy integrated.

Moreover, ddPCR was used to verify that the protospacer arrays in the four LPPs (PS-59) were no longer present additionally the LPP were PCR amplified for nanopore sequenced to confirm copy number integrated on each of the LPP and verify the integrated expression cassette in those 80% correct transformants, which indicates that the repair and insertion was successful.

A fermentation on microscale was done by making a spore solution of the successful transformants, those were inoculated on MTP containing 180 pl of liquid sucrose media, urea and uridine and incubated for seven days at 30° C., after 24 hours of incubation 10 μl 10.75 concentration maltose was added, on day 7 SDS page gels were made and enzyme activity assay was run to verify the enzyme production and validating the activity on the produced enzyme.

### Example 11. Generation of a Multi-Copy Lipase Strain

For building the Aspergillus oryzae recipient strain AT6818 containing six LPPs, the AT5885 strain described in Example 1 comprising 4 LPPs was used as starting point. Two additional chromosomal loci were chosen for inserting LPP #5 and LPP #6 into AT5885. First, LPP #5 was inserted on chromosome 3 between gene AO090026000695 and AO090026000696 at the position 786 nucleotides upstream from the translation initiation codon of gene AO090026000695. Second, LPP #6 was inserted on chromosome 7 at the nprA locus. The final strain was named AT6818, see Table 7 for an overview.

The full sequences of the six LPP protospacer arrays have been listed as:
SEQ ID NO: 14 (LPP #1): the five protospacers PS-59, PS-73, PS-74, PS-75 and PS-76 for integration in the pepAb locus;
SEQ ID NO: 15 (LPP #2): the five protospacers PS-59, PS-73, PS-74, PS-75 and PS-11 for integration in the pepE locus;
SEQ ID NO: 16 (LPP #3): the five protospacers P-59, PS-73, PS-48, PS-49 and PS-93 for integration in the amdS locus
SEQ ID NO: 17 (LPP #4): the five protospacers P-59, PS-73, PS-48, PS-89 and PS-94 for integration in the pepAa locus.
SEQ ID NO: 33 (LPP #5): the five protospacers P-59, PS-73, PS-74, PS-79 and PS-80 for integration at the A0090026000695 locus.
SEQ ID NO: 34 (LPP #6): the five protospacers PS-59, PS-73, PS-48, PS-89 and PS-90 for integration in the nprA locus.

TABLE 7
Overview of strains, plasmids and PCR fragments (DNA fragments) used in Example 11.

| Host strains | LLPs |
|---|---|
| AT5883 | LPP#1 and LPP#4 |
| AT5885 | LPP#1, LPP#2, LPP#3 and LPP#4 |
| AT6818 | LPP#1, LPP#2, LPP#3, LPP#4, LPP#5 and LPP#6 |
| Plasmids | |
| pAT4859 | CRISPR plasmid (gRNA for cutting PS-59) |
| DNA Fragments | |
| (FIG. 4) | |
| 750-1 | C33RTT/SPAM750_1.FOR/SPAM750_2.REV (764 bp) |
| | The 750-1 fragment contains the Left SYN2 flanking region and was PCR |
| | amplified from a plasmid containing the SYN2 flanking regions. The resulting |
| | 761 bp fragment was amplified using the primers 750_1FOR (SEQ ID NO: 35) |
| | and 750_2REV (SEQ ID NO: 36). |
| 750-2 | D36X6D(1)/SPAM750_3.FOR/SPAM750_4.REV (2398 bp) |
| | The 750-2 fragment contains the NA2 promoter, a lipase gene as well as a |
| | Terminator. The 2412 bp Linear DNA-fragment was amplified from a plasmid |
| | containing the Lipase expression cassette. The primers used for amplification |
| | was 750_3FW (SEQ ID NO: 37) and 750_4REV (SEQ ID NO: 38). |
| 750-3 | C33RTT/SPAM750_5.FOR/SPAM750_6.REV (761 bp) |
| | The right SYN2 flank was amplified from a plasmid containing the SYN2 flanks |
| | using the primers 750_5FW (SEQ ID NO: 39) and 750_6REV (SEQ ID NO: 40). |

The primers were designed to include overlapping regions to allow for combination and integration of the 3 PCR-fragments in SYN2 landing pads in the A. oryzae genome. The design is based on the method disclosed in WO2020025357A1 (Novozymes A/S).

The 750_2rev primer contain a 14 bp overlap to the lipase expression cassette in the 750-2 PCR fragment. The 750_3FW primer contain a 27 bp extension that overlaps with the 750-1 PCR fragment. Together the overlapping primers generate a 41 bp overlap between the PCR fragments, 750-1 and 750-2.

The 750_4 primer contains a 11 bp overlap with the 750-3 fragment and the 750_5 primer contains a 26 bp extension that overlaps with the 750-2 PCR-fragment. Together the overlapping primers generate a 37 bp overlap between the PCR fragments 750-2 and 750-3.

Protoplasts of the host strains listed in Table 7 were transformed with the CRISPR plasmid pAT4859 and three repair DNA fragments encoding SYN2-Left and SYN2-Right flanking regions homologous to the LPPs and a lipase TLL12225A expression cassette in between (FIG. 4). In FIG. 4 the dashed lines represent in-vivo homologous recombination between the different PCR fragments. The transformations were performed manually using PEG. The transformants were plated onto sucrose plates containing the appropriate nitrogen source (nitrate for AT5885 and AT6818, and urea for AT5883) and top agar was added to the transformants. After 4 days of incubation at 30° C. selected transformants were streak-purified twice and the integration of the expression cassette into the targeted LPPs was confirmed by ddPCR. In this way, the lipase strains shown in Table 8 were generated.

As can be seen from Table 8, using the SPADE system of the invention, with a single round of transformation of the PCR fragments we generated strains with 2, 4, and 6 copies of the lipase gene to occupy all LPPs of the host strains with the lipase genes. This example further demonstrates the efficient and versatile application of the SPADE system. Also, Example 11 shows that the SPADE system is not limited to be used with (linearized) plasmids, but can also be used with PCR fragments comprising the different features of the SPADE system, to further streamline strain generation.

TABLE 8
Strains generated with SPADE and single
transformation of PCR fragments.

| Strain name | Description |
|---|---|
| AT6842 | AT6818 + 6 copy TLL12225A |
| AT6843 | AT6818 + 6 copy TLL12225A |
| AT6844 | AT6818 + 6 copy TLL12225A |
| AT6845 | AT6818 + 6 copy TLL12225A |
| AT6848 | AT5883 + 2 copy TLL12225A |
| AT6849 | AT5885 + 4 copy TLL12225A |

### Example 12. Two Step Generation of Multiplex Strains Using Internal Gene Transfer

For building the Aspergillus oryzae recipient strain, the AT5885 strain was deleted in the three genes acvA, ipnA and aatA to remove the strains' ability to produce Penicillin. The resulting Aspergillus oryzae recipient strain was named AT6230.

A guide plasmid pAT4848, comprising gRNA sequence (SEQ ID NO: 13) enabling cutting of PS-48 present in two LPP (LPP #3 and LPP #4) of the four LPPs in AT6230 was made.

A repair DNA construct, REPAIR-14 (=first donor polynucleotide), encoding two copies of Bovine Alpha-Lactalbumin flanked by the repair flanks SYN2L and SYN2R was made.

A repair DNA construct, REPAIR-15 (=second donor polynucleotide), encoding one copy of the protein disulfide isomerase gene pdiA flanked by the repair flanks SYN2L and SYN2R was made.

In step1, the guide plasmid pAT4848 was transformed together with REPAIR-14 into AT6230 obtaining a strain A2F0285 having four copies of the Alpha-Lactalbumin gene with two copies inserted in LPP #3 and two copies inserted in LPP #4.

In step2, the guide plasmid pAT4873 was transformed together with REPAIR-15 into A2F0285. 20 Transformants were tested by ddPCR counting the number of Alpha-Lactalbumin genes and pdiA genes.

Using this procedure, we obtained three different outcome strains:
a. 8 copies of Alpha-Lactalbumin, 0 copies of pdiA.
b. 6 copies of Alpha-Lactalbumin, 1 copy of pdiA.
c. 4 copies of Alpha-Lactalbumin, 2 copies of pdiA.

Using internal gene copying mechanisms from one set of homology flanks to another, it was possible to obtain strains with varying ratios of alpha-lactalbumin and pdiA.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:
1. A host cell comprising in its genome at least two landing pad polynucleotide (LPP) sequences, each LPP sequence comprising in 5′ to 3′ direction:
   a 5′ homology arm,
   two or more protospacer sequences, and
   a 3′ homology arm,
wherein the polynucleotide sequence of the 5′ homology arm is identical for at least two LPP sequences, and
wherein the polynucleotide sequence of the 3′ homology arm is identical for at least two LPP sequences.
2. The host cell according to any one of the previous paragraphs, wherein at least one protospacer of the at least two protospacers is present in at least two LPP.
2a. The host cell according to any one of the previous paragraphs, wherein the cell comprises in its genome at least three LPP, and wherein at least one LPP comprises second 3′ and second 5′ homology arms having less than 100% sequence identity to the 3′ and 5′ homology arms of the at least two LPP, respectively, e.g., less than 99%, less than 98%, less than 97%, less than 96%, less than 95%, less than 90%, less thank 85%, less than 80%, less than 75%, less than 70%, or less than 65%.
3. The host cell according to any one of the previous paragraphs, wherein the polynucleotide sequence of the 5′ homology arm is identical for all of the at least two LPP sequences.
4. The host cell according to any one of the previous paragraphs, wherein the polynucleotide sequence of the 3′ homology arm is identical for all of the at least two LPP sequences.
5. The host cell according to any one of the previous paragraphs, wherein the 3′ homology arms and the 5′ homology arms are heterologous to the host cell.
6a. The host cell according to any one of the previous paragraphs, wherein the 3′ homology arms and the 5′ homology arms are synthetic polynucleotide sequences.
6b. The host cell according to any one of the previous paragraphs, wherein at least one LPP is located on the coding strand (3′ to 5′ direction), and wherein at least one LPP is located on the template strand (5′ to 3′ direction).
6c. The host cell according to any one of the previous paragraphs, wherein at least two LPPs are located on the coding strand (3′ to 5′ direction), and wherein at least two LPPs are located on the template strand (5′ to 3′ direction).
7. The host cell according to any one of the previous paragraphs, wherein each LPP comprises two protospacer sequences, such as a first protospacer, and a second protospacer.
8. The host cell according to any one of the previous paragraphs, wherein each LPP comprises three different protospacer sequences, such as a first protospacer, a second protospacer, and a third protospacer.
9. The host cell according to any one of the previous paragraphs, wherein each LPP comprises four different protospacer sequences, such as a first protospacer, a second protospacer, a third protospacer, and a fourth protospacer.
10. The host cell according to any one of the previous paragraphs, wherein each LPP comprises five different protospacer sequences, such as a first protospacer, a second protospacer, a third protospacer, a fourth protospacer, and a fifth protospacer.
11. The host cell according to any one of the previous paragraphs, wherein the first protospacer has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the second protospacer, to the polynucleotide sequence of the third protospacer, to the polynucleotide sequence of the fourth protospacer, and to the polynucleotide sequence of the fifth protospacer.
12. The host cell according to any one of the previous paragraphs, wherein the second protospacer has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the first protospacer, to the polynucleotide sequence of the third protospacer, to the polynucleotide sequence of the fourth protospacer, and to the polynucleotide sequence of the fifth protospacer.
13. The host cell according to any one of the previous paragraphs, wherein the third protospacer has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the first protospacer, to the polynucleotide sequence of the second protospacer, to the polynucleotide sequence of the fourth protospacer, and to the polynucleotide sequence of the fifth protospacer.
14. The host cell according to any one of the previous paragraphs, wherein the fourth protospacer has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the first protospacer, to the polynucleotide sequence of the second protospacer, to the polynucleotide sequence of the third protospacer, and to the polynucleotide sequence of the fifth protospacer.
15. The host cell according to any one of the previous paragraphs, wherein the fifth protospacer has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the first protospacer, to the polynucleotide sequence of the second protospacer, to the polynucleotide sequence of the third protospacer, and to the polynucleotide sequence of the fourth protospacer.
16. The host cell according to any one of the previous paragraphs, wherein each LPP comprises more than five different protospacer sequences.
17. The host cell according to any one of the previous paragraphs, wherein one or more of the at least two protospacer sequences is heterologous to the host cell.
18. The host cell according to any one of the previous paragraphs, wherein all of the at least two protospacer sequences are heterologous to the host cell.
19. The host cell according to any one of the previous paragraphs, wherein one or more of the at least two protospacer sequences is synthetic.
20. The host cell according to any one of the previous paragraphs, wherein all of the at least two protospacer sequences is synthetic.
21. The host cell according to any one of the previous paragraphs, wherein each protospacer sequence has a length of at least 15 nt, at least 20 nt, or at least 25 nt, preferably each protospacer has a length of 20-30 nt.
22. The host cell according to any one of the previous paragraphs, wherein the host cell comprises two LPP sequences, three LPP sequences, four LPP sequences, 5 LPP sequences, 6 LPP sequences, 7 LPP sequences, 8 LPP sequences, 9 LPP sequences, 10 LPP sequences, 11 LPP sequences, 12 LPP sequences, 13 LPP sequences, 14 LPP sequences, 15 LPP sequences, 16 LPP sequences, or more than 16 LPP sequences.
23. The host cell according to any one of the previous paragraphs, wherein the host cell comprises 2-8 LPP sequences.
24. The host cell according to any one of the previous paragraphs, wherein the host cell comprises at least two chromosomes, at least four chromosomes, at least six chromosomes or at least 8 chromosomes.
25. The host cell according to any one of the previous paragraphs, wherein one or more of the host cell chromosomes comprises a total of 1 LPP per chromosome.
26. The host cell according to any one of the previous paragraphs, wherein two or more chromosomes comprise a total of 1 LPP per chromosome.
27. The host cell according to any one of the previous paragraphs, wherein three chromosomes comprise a total of 1 LPP per chromosome.
28. The host cell according to any one of the previous paragraphs, wherein four chromosomes comprise a total of 1 LPP per chromosome.
29. The host cell according to any one of the previous paragraphs, wherein five chromosomes comprise a total of 1 LPP per chromosome.
30. The host cell according to any one of the previous paragraphs, wherein six chromosomes comprise a total of 1 LPP per chromosome.
31. The host cell according to any one of the previous paragraphs, wherein 7 chromosomes comprise a total of 1 LPP per chromosome.
32. The host cell according to any one of the previous paragraphs, wherein 8 chromosomes comprise a total of 1 LPP per chromosome.
33. The host cell according to any one of the previous paragraphs, wherein each chromosome comprises a total of 1 LPP per chromosome.
34. The host cell according to any one of the previous paragraphs, wherein the LPP is located on the short arm of a chromosome.
35. The host cell according to any one of the previous paragraphs, wherein the LPP is located on the long arm of a chromosome.
36. The host cell according to any one of the previous paragraphs, wherein one or more of the chromosomes comprises a total of 2 LPP per chromosome.
37. The host cell according to any one of the previous paragraphs, wherein two or more chromosomes comprise a total of 2 LPP per chromosome.
38. The host cell according to any one of the previous paragraphs, wherein three chromosomes comprise a total of 2 LPP per chromosome.
39. The host cell according to any one of the previous paragraphs, wherein four chromosomes comprise a total of 2 LPP per chromosome.
40. The host cell according to any one of the previous paragraphs, wherein five chromosomes comprise a total of 2 LPP per chromosome.
41. The host cell according to any one of the previous paragraphs, wherein six chromosomes comprise a total of 2 LPP per chromosome.
42. The host cell according to any one of the previous paragraphs, wherein 7 chromosomes comprise a total of 2 LPP per chromosome.
43. The host cell according to any one of the previous paragraphs, wherein 8 chromosomes comprise a total of 2 LPP per chromosome.
44. The host cell according to any one of the previous paragraphs, wherein each chromosome comprises a total of 2 LPP per chromosome.
45. The host cell according to any one of the previous paragraphs, wherein for each of the one or more chromosomes, the 2 LPP are located on opposite locations of the centromere of the chromosome.
46. The host cell according to any one of the previous paragraphs, wherein one LPP is located on the short arm, and one LPP is located on the long arm.
47. The host cell according to any one of the previous paragraphs, wherein one or more of the chromosomes comprises a total of 3 LPP per chromosome.
48. The host cell according to any one of the previous paragraphs, wherein two or more chromosomes comprise a total of 3 LPP per chromosome.
49. The host cell according to any one of the previous paragraphs, wherein three chromosomes comprise a total of 3 LPP per chromosome.
50. The host cell according to any one of the previous paragraphs, wherein four chromosomes comprise a total of 3 LPP per chromosome.
51. The host cell according to any one of the previous paragraphs, wherein five chromosomes comprise a total of 3 LPP per chromosome.
52. The host cell according to any one of the previous paragraphs, wherein six chromosomes comprise a total of 3 LPP per chromosome.
53. The host cell according to any one of the previous paragraphs, wherein 7 chromosomes comprise a total of 3 LPP per chromosome.
54. The host cell according to any one of the previous paragraphs, wherein 8 chromosomes comprise a total of 3 LPP per chromosome.
55. The host cell according to any one of the previous paragraphs, wherein each chromosome comprises a total of 3 LPP per chromosome.
56. The host cell according to any one of the previous paragraphs, comprising 3 LPP per chromosome, the ratio of numbers of LPP between short arm and long arm is selected from the list of 3:0, 0:3, 2:1, and 1:2.
57. The host cell according to any one of the previous paragraphs, wherein one or more of the chromosomes comprises a total of 4 LPP per chromosome.
58. The host cell according to any one of the previous paragraphs, wherein two or more chromosomes comprise a total of 4 LPP per chromosome.
59. The host cell according to any one of the previous paragraphs, wherein three chromosomes comprise a total of 4 LPP per chromosome.
60. The host cell according to any one of the previous paragraphs, wherein four chromosomes comprise a total of 4 LPP per chromosome.
61. The host cell according to any one of the previous paragraphs, wherein five chromosomes comprise a total of 4 LPP per chromosome.
62. The host cell according to any one of the previous paragraphs, wherein six chromosomes comprise a total of 4 LPP per chromosome.
63. The host cell according to any one of the previous paragraphs, wherein 7 chromosomes comprise a total of 4 LPP per chromosome.
64. The host cell according to any one of the previous paragraphs, wherein 8 chromosomes comprise a total of 4 LPP per chromosome.
65. The host cell according to any one of the previous paragraphs, wherein each chromosome comprises a total of 4 LPP per chromosome.
66. The host cell according to any one of the previous paragraphs, wherein for 4 LPP per chromosome, the ratio of numbers of LPP between short arm and long arm is selected from the list of 4:0, 0:4, 3:1, 1:3, 2:2, 2:1, and 1:2.
67. The host cell according to any one of the previous paragraphs, wherein the host cell is deficient of a non-homologous end joining (NHEJ) mechanism.
68. The host cell according to any one of the previous paragraphs, wherein each 3′ homology arm and each 5′ homology arm has a length of at least 10 nt, at least 20 nt, at least 30 nt, at least 50 nt, at least 100 nt, 200 nucleotides (nt), at least 300 nt, at least 400 nt, at least 500 nt, at least 600 nt, at least 700 nt, at least 800 nt, at least 900 nt, or at least 1000 nt.
69. The host cell according to any one of the previous paragraphs, wherein each 3′ homology arm and each 5′ homology arm has a length of at least 100 nt.
70. The host cell according to any one of the previous paragraphs, wherein each 3′ homology arm and each 5′ homology arm has a length of at least 800 nt, at least 900 nt, or at least 1000 nt.
71. The host cell according to any one of the previous paragraphs, wherein one or more LPP of the at least two LPP comprise a selectable marker, and/or a counter-selectable marker.
72. The host cell according to any one of the previous paragraphs, wherein 2 or more LPP of the at least two LPP comprise a selectable marker, and/or a counter-selectable marker.
73. The host cell according to any one of the previous paragraphs, wherein 3 or more LPP of the at least two LPP comprise a selectable marker, and/or a counter-selectable marker.
74. The host cell according to any one of the previous paragraphs, wherein 4 or more LPP of the at least two LPP comprise a selectable marker, and/or a counter-selectable marker.
75. The host cell according to any one of the previous paragraphs, wherein 5 or more LPP of the at least two LPP comprise a selectable marker, and/or a counter-selectable marker.
76. The host cell according to any one of the previous paragraphs, wherein 6 or more LPP of the at least two LPP comprise a selectable marker, and/or a counter-selectable marker.
77. The host cell according to any one of the previous paragraphs, wherein 7 or more LPP of the at least two LPP comprise a selectable marker, and/or a counter-selectable marker.
78. The host cell according to any one of the previous paragraphs, wherein 8 or more LPP of the at least two LPP comprise a selectable marker, and/or a counter-selectable marker.
79. The host cell according to any one of the previous paragraphs, wherein the selectable marker comprises a marker selected from the list of a pyrG gene, a fcy1 gene, an adeA gene, an adeB gene, a metF gene, a metG gene, a metH gene, a lysF gene, a trpC gene, a sC gene, an argB gene, and a gene encoding a fluorescent protein, such as a GFP, YFP, CFP, DsRed, or a eqPF611.
80. The host cell according to any one of the previous paragraphs, wherein the counter-selectable marker comprises a marker selected from the list of a pyrG gene, a fcy1 gene, an adeA gene, an adeB gene, a metF gene, a metG gene, a metH gene, a lysF gene, a trpC gene, a sC gene, an argB gene, and a gene encoding a fluorescent protein, such as a GFP, YFP, CFP, DsRed, or a eqPF611.
81. The host cell according to any one of the previous paragraphs, wherein none of the at least two LPP comprises a selectable marker, and/or a counter-selectable marker.
82. The host cell according to any one of the previous paragraphs, wherein each DNA strain complementary to the two or more protospacers sequences comprises a PAM sequence (protospacer adjacent motif).
83. The host cell according to any one of the previous paragraphs, wherein the PAM sequence is selected from the list of “NGG”, “NGRRT”, “NGRRN”, “NNNNGATT”, “NNNNRYAC”, “NNAGAAW”, “TTTV”, “TTTC”, “TTTN”, “TTN”, “ATTN”, “GTTN”, and “CTTN” (“N” being any nucleotide, “Y”=C or T, “V”=A or C or G, “W”=A or T, “R”=A or G), preferably the PAM sequence is TTTN, more preferably the PAM sequence is TTTC.
84. The host cell according to any one of the previous paragraphs, wherein the first, second, third, fourth or fifth protospacer sequence is selected from a sequence having a sequence identity of at least 80%, at least 85%, at least 90%, at least 95%, at least 96%, at least 97%, at least 98%, at least 99%, or 100% to SEQ ID NO: 3, SEQ ID NO: 4, SEQ ID NO: 5, SEQ ID NO: 6, SEQ ID NO: 7, SEQ ID NO: 8, SEW ID NO: 9, SEQ ID NO: 10, SEQ ID NO: 11, SEQ ID NO: 12, or SEQ ID NO: 13.
85. The host cell according to any one of the previous paragraphs, wherein the 5′ homology arm sequence comprises or consists of SEQ ID NO: 1 or SEQ ID NO: 2.
85a. The host cell according to any one of the previous paragraphs, wherein the 5′ homology arm sequences comprises or consists of a polynucleotide sequence having a sequence identity of at least 80%, at least 85%, at least 90%, at least 95%, at least 96%, at least 97%, at least 98%, at least 99%, or 100% to SEQ ID NO: 1 or SEQ ID NO: 2.
86. The host cell according to any one of the previous paragraphs, wherein the 3′ homology arm sequence comprises or consists of SEQ ID NO: 1 or SEQ ID NO: 2.
86a. The host cell according to any one of the previous paragraphs, wherein the 3′ homology arm sequences comprises or consists of a polynucleotide sequence having a sequence identity of at least 80%, at least 85%, at least 90%, at least 95%, at least 96%, at least 97%, at least 98%, at least 99%, or 100% to SEQ ID NO: 1 or SEQ ID NO: 2.
87. The host cell according to any one of the previous paragraphs, wherein each of the at least two LPP comprises a first protospacer sequence with a polynucleotide sequence identical for each first protospacer of each at least two LPP.
88. The host cell according to any one of the previous paragraphs, wherein each of the at least two LPP comprises a second protospacer sequence with a polynucleotide sequence which is unique for each second protospacer of each at least two LPP.
89. The host cell according to any one of the previous paragraphs, wherein each of the at least two LPP comprises a third protospacer sequence with a polynucleotide sequence which is identical for each third polynucleotide within a third subset of the at least two LPPs.
90. The host cell according to any one of the previous paragraphs, wherein the third subset consists of 2 LPP sequences, 3 LPP sequences, 4 LPP sequences, 5 LPP sequences, 6 LPP sequences, 7 LPP sequences, 8 LPP sequences, 9 LPP sequences, 10 LPP, sequences, 11 LPP sequences, 12 LPP sequences, 13 LPP sequences, 14 LPP sequences, or 15 LPP sequences.
91. The host cell according to any one of the previous paragraphs, wherein each of the at least two LPP comprises a fourth protospacer sequence with a polynucleotide sequence which is identical for each fourth polynucleotide within a fourth subset of the at least two LPPs.
92. The host cell according to any one of the previous paragraphs, wherein the fourth subset consists of 2 LPP sequences, 3 LPP sequences, 4 LPP sequences, 5 LPP sequences, 6 LPP sequences, 7 LPP sequences, 8 LPP sequences, 9 LPP sequences, 10 LPP, sequences, 11 LPP sequences, 12 LPP sequences, 13 LPP sequences, 14 LPP sequences, or 15 LPP sequences.
93. The host cell according to any one of the previous paragraphs, wherein each of the at least two LPP comprises a fifth protospacer sequence with a polynucleotide sequence which is identical for each fifth protospacer within a fifth subset of the at least two LPPs.
94. The host cell according to any one of the previous paragraphs, wherein the fifth subset consists of 2 LPP sequences, 3 LPP sequences, 4 LPP sequences, 5 LPP sequences, 6 LPP sequences, 7 LPP sequences, 8 LPP sequences, 9 LPP sequences, 10 LPP, sequences, 11 LPP sequences, 12 LPP sequences, 13 LPP sequences, 14 LPP sequences, or 15 LPP sequences.
95. The host cell according to any one of the previous paragraphs, wherein the host cell comprises three LPP sequences, and wherein the third, fourth, or fifth protospacer sequence is identical for two of the three LPPs.
96. The host cell according to any one of the previous paragraphs, wherein the host cell comprises four LPP sequences, and wherein the third, fourth, or fifth protospacer sequence is identical for two of the four LPPs.
97. The host cell according to any one of the previous paragraphs, wherein the host cell comprises four LPP sequences, and wherein the third, fourth, or fifth protospacer sequence is identical for three of the four LPPs.
98. The host cell according to any one of the previous paragraphs, wherein the host cell comprises five LPP sequences, and wherein the third, fourth, or fifth protospacer sequence is identical for two of the five LPPs.
99. The host cell according to any one of the previous paragraphs, wherein the host cell comprises five LPP sequences, and wherein the third, fourth, or fifth protospacer sequence is identical for three of the five LPPs.
100. The host cell according to any one of the previous paragraphs, wherein the host cell comprises five LPP sequences, and wherein the third, fourth, or fifth protospacer sequence is identical for four of the five LPPs.
101. The host cell according to any one of the previous paragraphs, wherein the host cell comprises six LPP sequences, and wherein the third, fourth, or fifth protospacer sequence is identical for two of the six LPPs.
102. The host cell according to any one of the previous paragraphs, wherein the host cell comprises six LPP sequences, and wherein the third, fourth, or fifth protospacer sequence is identical for three of the six LPPs.
103. The host cell according to any one of the previous paragraphs, wherein the host cell comprises six LPP sequences, and wherein the third, fourth, or fifth protospacer sequence is identical for four of the six LPPs.
104. The host cell according to any one of the previous paragraphs, wherein the host cell comprises six LPP sequences, and wherein the third, fourth, or fifth protospacer sequence is identical for five of the six LPPs.
105. The host cell according to any one of the previous paragraphs, wherein the host cell comprises 7 LPP sequences, and wherein the third, fourth, or fifth protospacer sequence is identical for two of the 7 LPPs.
106. The host cell according to any one of the previous paragraphs, wherein the host cell comprises 7 LPP sequences, and wherein the third, fourth, or fifth protospacer sequence is identical for three of the 7 LPPs.
107. The host cell according to any one of the previous paragraphs, wherein the host cell comprises 7 LPP sequences, and wherein the third, fourth, or fifth protospacer sequence is identical for four of the 7 LPPs.
108. The host cell according to any one of the previous paragraphs, wherein the host cell comprises 7 LPP sequences, and wherein the third, fourth, or fifth protospacer sequence is identical for five of the 7 LPPs.
109. The host cell according to any one of the previous paragraphs, wherein the host cell comprises 7 LPP sequences, and wherein the third, fourth, or fifth protospacer sequence is identical for six of the 7 LPPs.
110. The host cell according to any one of the previous paragraphs, wherein the host cell comprises 8 LPP sequences, and wherein the third, fourth, or fifth protospacer sequence is identical for two of the 8 LPPs.
111. The host cell according to any one of the previous paragraphs, wherein the host cell comprises 8 LPP sequences, and wherein the third, fourth, or fifth protospacer sequence is identical for three of the 8 LPPs.
112. The host cell according to any one of the previous paragraphs, wherein the host cell comprises 8 LPP sequences, and wherein the third, fourth, or fifth protospacer sequence is identical for four of the 8 LPPs.
113. The host cell according to any one of the previous paragraphs, wherein the host cell comprises 8 LPP sequences, and wherein the third, fourth, or fifth protospacer sequence is identical for five of the 8 LPPs.
114. The host cell according to any one of the previous paragraphs, wherein the host cell comprises 8 LPP sequences, and wherein the third, fourth, or fifth protospacer sequence is identical for six of the 8 LPPs.
115. The host cell according to any one of the previous paragraphs, wherein the host cell comprises 8 LPP sequences, and wherein the third, fourth, or fifth protospacer sequence is identical for 7 of the 8 LPPs.
116. The host cell according to any one of the previous paragraphs, wherein the host cell comprises 9 LPP sequences, and wherein the third, fourth, or fifth protospacer sequence is identical for two of the 9 LPPs.
117. The host cell according to any one of the previous paragraphs, wherein the host cell comprises 9 LPP sequences, and wherein the third, fourth, or fifth protospacer sequence is identical for three of the 9 LPPs.
118. The host cell according to any one of the previous paragraphs, wherein the host cell comprises 9 LPP sequences, and wherein the third, fourth, or fifth protospacer sequence is identical for four of the 9 LPPs.
119. The host cell according to any one of the previous paragraphs, wherein the host cell comprises 9 LPP sequences, and wherein the third, fourth, or fifth protospacer sequence is identical for five of the 9 LPPs.
120. The host cell according to any one of the previous paragraphs, wherein the host cell comprises 9 LPP sequences, and wherein the third, fourth, or fifth protospacer sequence is identical for six of the 9 LPPs.
121. The host cell according to any one of the previous paragraphs, wherein the host cell comprises 9 LPP sequences, and wherein the third, fourth, or fifth protospacer sequence is identical for 7 of the 9 LPPs.
122. The host cell according to any one of the previous paragraphs, wherein the host cell comprises 9 LPP sequences, and wherein the third, fourth, or fifth protospacer sequence is identical for 8 of the 9 LPPs.
123. The host cell according to any one of the previous paragraphs, wherein the host cell comprises 10 LPP sequences, and wherein the third, fourth, or fifth protospacer sequence is identical for two of the 10 LPPs.
124. The host cell according to any one of the previous paragraphs, wherein the host cell comprises 10 LPP sequences, and wherein the third, fourth, or fifth protospacer sequence is identical for three of the 10 LPPs.
125. The host cell according to any one of the previous paragraphs, wherein the host cell comprises 10 LPP sequences, and wherein the third, fourth, or fifth protospacer sequence is identical for four of the 10 LPPs.
126. The host cell according to any one of the previous paragraphs, wherein the host cell comprises 10 LPP sequences, and wherein the third, fourth, or fifth protospacer sequence is identical for five of the 10 LPPs.
127. The host cell according to any one of the previous paragraphs, wherein the host cell comprises 10 LPP sequences, and wherein the third, fourth, or fifth protospacer sequence is identical for six of the 10 LPPs.
128. The host cell according to any one of the previous paragraphs, wherein the host cell comprises 10 LPP sequences, and wherein the third, fourth, or fifth protospacer sequence is identical for 7 of the 10 LPPs.
129. The host cell according to any one of the previous paragraphs, wherein the host cell comprises 10 LPP sequences, and wherein the third, fourth, or fifth protospacer sequence is identical for 8 of the 10 LPPs.
130. The host cell according to any one of the previous paragraphs, wherein the host cell comprises 10 LPP sequences, and wherein the third, fourth, or fifth protospacer sequence is identical for 9 of the 10 LPPs.
131. The host cell according to any one of the previous paragraphs, wherein the host cell comprises 11 LPP sequences, and wherein the third, fourth, or fifth protospacer sequence is identical for two of the 11 LPPs.
132. The host cell according to any one of the previous paragraphs, wherein the host cell comprises 11 LPP sequences, and wherein the third, fourth, or fifth protospacer sequence is identical for three of the 11 LPPs.
134. The host cell according to any one of the previous paragraphs, wherein the host cell comprises 11 LPP sequences, and wherein the third, fourth, or fifth protospacer sequence is identical for four of the 11 LPPs.
135. The host cell according to any one of the previous paragraphs, wherein the host cell comprises 11 LPP sequences, and wherein the third, fourth, or fifth protospacer sequence is identical for five of the 11 LPPs.
136. The host cell according to any one of the previous paragraphs, wherein the host cell comprises 11 LPP sequences, and wherein the third, fourth, or fifth protospacer sequence is identical for six of the 11 LPPs.
137. The host cell according to any one of the previous paragraphs, wherein the host cell comprises 11 LPP sequences, and wherein the third, fourth, or fifth protospacer sequence is identical for 7 of the 11 LPPs.
138. The host cell according to any one of the previous paragraphs, wherein the host cell comprises 11 LPP sequences, and wherein the third, fourth, or fifth protospacer sequence is identical for 8 of the 11 LPPs.
139. The host cell according to any one of the previous paragraphs, wherein the host cell comprises 11 LPP sequences, and wherein the third, fourth, or fifth protospacer sequence is identical for 9 of the 11 LPPs.
140. The host cell according to any one of the previous paragraphs, wherein the host cell comprises 11 LPP sequences, and wherein the third, fourth, or fifth protospacer sequence is identical for 10 of the 11 LPPs.
141. The host cell according to any one of the previous paragraphs, wherein the host cell comprises 12 LPP sequences, and wherein the third, fourth, or fifth protospacer sequence is identical for two of the 12 LPPs.
142. The host cell according to any one of the previous paragraphs, wherein the host cell comprises 12 LPP sequences, and wherein the third, fourth, or fifth protospacer sequence is identical for three of the 12 LPPs.
143. The host cell according to any one of the previous paragraphs, wherein the host cell comprises 12 LPP sequences, and wherein the third, fourth, or fifth protospacer sequence is identical for four of the 12 LPPs.
144. The host cell according to any one of the previous paragraphs, wherein the host cell comprises 12 LPP sequences, and wherein the third, fourth, or fifth protospacer sequence is identical for five of the 12 LPPs.
145. The host cell according to any one of the previous paragraphs, wherein the host cell comprises 12 LPP sequences, and wherein the third, fourth, or fifth protospacer sequence is identical for six of the 12 LPPs.
146. The host cell according to any one of the previous paragraphs, wherein the host cell comprises 12 LPP sequences, and wherein the third, fourth, or fifth protospacer sequence is identical for 7 of the 12 LPPs.
147. The host cell according to any one of the previous paragraphs, wherein the host cell comprises 12 LPP sequences, and wherein the third, fourth, or fifth protospacer sequence is identical for 8 of the 12 LPPs.
148. The host cell according to any one of the previous paragraphs, wherein the host cell comprises 12 LPP sequences, and wherein the third, fourth, or fifth protospacer sequence is identical for 9 of the 12 LPPs.
149. The host cell according to any one of the previous paragraphs, wherein the host cell comprises 12 LPP sequences, and wherein the third, fourth, or fifth protospacer sequence is identical for 10 of the 12LPPs.
150. The host cell according to any one of the previous paragraphs, wherein the host cell comprises 12 LPP sequences, and wherein the third, fourth, or fifth protospacer sequence is identical for 11 of the 12 LPPs.
151. The host cell according to any one of the previous paragraphs, wherein the host cell comprises 13 LPP sequences, and wherein the third, fourth, or fifth protospacer sequence is identical for two of the 13 LPPs.
152. The host cell according to any one of the previous paragraphs, wherein the host cell comprises 13 LPP sequences, and wherein the third, fourth, or fifth protospacer sequence is identical for three of the 13 LPPs.
153. The host cell according to any one of the previous paragraphs, wherein the host cell comprises 13 LPP sequences, and wherein the third, fourth, or fifth protospacer sequence is identical for four of the 13 LPPs.
154. The host cell according to any one of the previous paragraphs, wherein the host cell comprises 13 LPP sequences, and wherein the third, fourth, or fifth protospacer sequence is identical for five of the 13 LPPs.
155. The host cell according to any one of the previous paragraphs, wherein the host cell comprises 13 LPP sequences, and wherein the third, fourth, or fifth protospacer sequence is identical for six of the 13 LPPs.
156. The host cell according to any one of the previous paragraphs, wherein the host cell comprises 13 LPP sequences, and wherein the third, fourth, or fifth protospacer sequence is identical for 7 of the 13 LPPs.
157. The host cell according to any one of the previous paragraphs, wherein the host cell comprises 13 LPP sequences, and wherein the third, fourth, or fifth protospacer sequence is identical for 8 of the 13 LPPs.
158. The host cell according to any one of the previous paragraphs, wherein the host cell comprises 13 LPP sequences, and wherein the third, fourth, or fifth protospacer sequence is identical for 9 of the 13 LPPs.
159. The host cell according to any one of the previous paragraphs, wherein the host cell comprises 13 LPP sequences, and wherein the third, fourth, or fifth protospacer sequence is identical for 10 of the 13 LPPs.
160. The host cell according to any one of the previous paragraphs, wherein the host cell comprises 13 LPP sequences, and wherein the third, fourth, or fifth protospacer sequence is identical for 11 of the 13 LPPs.
161. The host cell according to any one of the previous paragraphs, wherein the host cell comprises 13 LPP sequences, and wherein the third, fourth, or fifth protospacer sequence is identical for 12 of the 13 LPPs.
162. The host cell according to any one of the previous paragraphs, wherein the host cell comprises 14 LPP sequences, and wherein the third, fourth, or fifth protospacer sequence is identical for two of the 14 LPPs.
163. The host cell according to any one of the previous paragraphs, wherein the host cell comprises 14 LPP sequences, and wherein the third, fourth, or fifth protospacer sequence is identical for three of the 14 LPPs.
164. The host cell according to any one of the previous paragraphs, wherein the host cell comprises 14 LPP sequences, and wherein the third, fourth, or fifth protospacer sequence is identical for four of the 14 LPPs.
165. The host cell according to any one of the previous paragraphs, wherein the host cell comprises 14 LPP sequences, and wherein the third, fourth, or fifth protospacer sequence is identical for five of the 14 LPPs.
166. The host cell according to any one of the previous paragraphs, wherein the host cell comprises 14 LPP sequences, and wherein the third, fourth, or fifth protospacer sequence is identical for six of the 14 LPPs.
167. The host cell according to any one of the previous paragraphs, wherein the host cell comprises 14 LPP sequences, and wherein the third, fourth, or fifth protospacer sequence is identical for 7 of the 14 LPPs.
168. The host cell according to any one of the previous paragraphs, wherein the host cell comprises 14 LPP sequences, and wherein the third, fourth, or fifth protospacer sequence is identical for 8 of the 14 LPPs.
169. The host cell according to any one of the previous paragraphs, wherein the host cell comprises 14 LPP sequences, and wherein the third, fourth, or fifth protospacer sequence is identical for 9 of the 14 LPPs.
170. The host cell according to any one of the previous paragraphs, wherein the host cell comprises 14 LPP sequences, and wherein the third, fourth, or fifth protospacer sequence is identical for 10 of the 14 LPPs.
171. The host cell according to any one of the previous paragraphs, wherein the host cell comprises 14 LPP sequences, and wherein the third, fourth, or fifth protospacer sequence is identical for 11 of the 14 LPPs.
172. The host cell according to any one of the previous paragraphs, wherein the host cell comprises 14 LPP sequences, and wherein the third, fourth, or fifth protospacer sequence is identical for 12 of the 14 LPPs.
173. The host cell according to any one of the previous paragraphs, wherein the host cell comprises 14 LPP sequences, and wherein the third, fourth, or fifth protospacer sequence is identical for 13 of the 14 LPPs.
174. The host cell according to any one of the previous paragraphs, wherein the host cell comprises 15 LPP sequences, and wherein the third, fourth, or fifth protospacer sequence is identical for two of the 15 LPPs.
175. The host cell according to any one of the previous paragraphs, wherein the host cell comprises 15 LPP sequences, and wherein the third, fourth, or fifth protospacer sequence is identical for three of the 15 LPPs.
176. The host cell according to any one of the previous paragraphs, wherein the host cell comprises 15 LPP sequences, and wherein the third, fourth, or fifth protospacer sequence is identical for four of the 15 LPPs.
177. The host cell according to any one of the previous paragraphs, wherein the host cell comprises 15 LPP sequences, and wherein the third, fourth, or fifth protospacer sequence is identical for five of the 15 LPPs.
178. The host cell according to any one of the previous paragraphs, wherein the host cell comprises 15 LPP sequences, and wherein the third, fourth, or fifth protospacer sequence is identical for six of the 15 LPPs.
179. The host cell according to any one of the previous paragraphs, wherein the host cell comprises 15 LPP sequences, and wherein the third, fourth, or fifth protospacer sequence is identical for 7 of the 15 LPPs.
180. The host cell according to any one of the previous paragraphs, wherein the host cell comprises 15 LPP sequences, and wherein the third, fourth, or fifth protospacer sequence is identical for 8 of the 15 LPPs.
181. The host cell according to any one of the previous paragraphs, wherein the host cell comprises 15 LPP sequences, and wherein the third, fourth, or fifth protospacer sequence is identical for 9 of the 15 LPPs.
182. The host cell according to any one of the previous paragraphs, wherein the host cell comprises 15 LPP sequences, and wherein the third, fourth, or fifth protospacer sequence is identical for 10 of the 15 LPPs.
183. The host cell according to any one of the previous paragraphs, wherein the host cell comprises 15 LPP sequences, and wherein the third, fourth, or fifth protospacer sequence is identical for 11 of the 15 LPPs.
184. The host cell according to any one of the previous paragraphs, wherein the host cell comprises 15 LPP sequences, and wherein the third, fourth, or fifth protospacer sequence is identical for 12 of the 15 LPPs.
185. The host cell according to any one of the previous paragraphs, wherein the host cell comprises 15 LPP sequences, and wherein the third, fourth, or fifth protospacer sequence is identical for 13 of the 15 LPPs.
186. The host cell according to any one of the previous paragraphs, wherein the host cell comprises 15 LPP sequences, and wherein the third, fourth, or fifth protospacer sequence is identical for 14 of the 15 LPPs.
187. The host cell according to any one of the previous paragraphs, wherein the host cell comprises 16 LPP sequences, and wherein the third, fourth, or fifth protospacer sequence is identical for two of the 16 LPPs.
188. The host cell according to any one of the previous paragraphs, wherein the host cell comprises 16 LPP sequences, and wherein the third, fourth, or fifth protospacer sequence is identical for three of the 16 LPPs.
189. The host cell according to any one of the previous paragraphs, wherein the host cell comprises 16 LPP sequences, and wherein the third, fourth, or fifth protospacer sequence is identical for four of the 16 LPPs.
190. The host cell according to any one of the previous paragraphs, wherein the host cell comprises 16 LPP sequences, and wherein the third, fourth, or fifth protospacer sequence is identical for five of the 16 LPPs.
191. The host cell according to any one of the previous paragraphs, wherein the host cell comprises 16 LPP sequences, and wherein the third, fourth, or fifth protospacer sequence is identical for six of the 16 LPPs.
192. The host cell according to any one of the previous paragraphs, wherein the host cell comprises 16 LPP sequences, and wherein the third, fourth, or fifth protospacer sequence is identical for 7 of the 16 LPPs.
193. The host cell according to any one of the previous paragraphs, wherein the host cell comprises 16 LPP sequences, and wherein the third, fourth, or fifth protospacer sequence is identical for 8 of the 16 LPPs.
194. The host cell according to any one of the previous paragraphs, wherein the host cell comprises 16 LPP sequences, and wherein the third, fourth, or fifth protospacer sequence is identical for 9 of the 16 LPPs.
195. The host cell according to any one of the previous paragraphs, wherein the host cell comprises 16 LPP sequences, and wherein the third, fourth, or fifth protospacer sequence is identical for 10 of the 16 LPPs.
196. The host cell according to any one of the previous paragraphs, wherein the host cell comprises 16 LPP sequences, and wherein the third, fourth, or fifth protospacer sequence is identical for 11 of the 16 LPPs.
197. The host cell according to any one of the previous paragraphs, wherein the host cell comprises 16 LPP sequences, and wherein the third, fourth, or fifth protospacer sequence is identical for 12 of the 16 LPPs.
198. The host cell according to any one of the previous paragraphs, wherein the host cell comprises 16 LPP sequences, and wherein the third, fourth, or fifth protospacer sequence is identical for 13 of the 16 LPPs.
199. The host cell according to any one of the previous paragraphs, wherein the host cell comprises 16 LPP sequences, and wherein the third, fourth, or fifth protospacer sequence is identical for 14 of the 16 LPPs.
200. The host cell according to any one of the previous paragraphs, wherein the host cell comprises 16 LPP sequences, and wherein the third, fourth, or fifth protospacer sequence is identical for 15 of the 16 LPPs.
201. The host cell according to any one of the previous paragraphs, wherein the host cell is a fungal cell.
202. The host cell according to any one of the previous paragraphs, wherein the host cell is a yeast recombinant host cell, e.g., a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.
203. The host cell according to any one of the previous paragraphs, wherein the host cell is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.
204. The host cell according to any one of the previous paragraphs, wherein the host cell is an Aspergillus cell, preferably an Aspergillus niger cell, or an Aspergillus oryzae cell.
204a. The host cell according to any one of the previous paragraphs, wherein the host cell is a Trichoderma cell, preferably a Trichderma reesei cell.
205. The host cell according to any one of the previous paragraphs, which is isolated.
206. The host cell according to any one of the previous paragraphs, which is purified.
207. The host cell according to any one of the previous paragraphs, wherein the host cell is a prokaryotic recombinant host cell, e.g., a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, or a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.
208. The host cell according to any one of the previous paragraphs, wherein the host cell is a Bacillus cell.
209. The host cell according to any one of the previous paragraphs, wherein the host cell is a Bacillus licheniformis cell.
210. The host cell according to any one of the previous paragraphs, wherein the host cell is a Bacillus subtilis cell.
211. The host cell according to any one of the previous paragraphs, wherein the host cell is a prokaryotic host cell and wherein at least one LPP is located between two selectable and/or counter-selectable marker genes.
212. The host cell according to any one of the previous paragraphs, wherein the host cell is a mammalian cell.
213. The host cell according to any one of the previous paragraphs, wherein the mammalian cell is selected from the list of a human cell, a mouse cell, a rat cell, a mouse hybridoma cell, a hamster cell (e.g. Chinese hamster ovary cell), and a rat hybridoma cell.
214. A method for generating a multi-copy host cell, comprising the steps of
a) providing a host cell according to any one of the previous paragraphs,
b) delivering to the host cell a gene editing system comprising:
   a RNA-guided DNA nuclease,
   at least one guide RNA directed to one or more protospacer of the one or more LPP sequences,
   a first donor polynucleotide comprising in 5′ to 3′ direction: a 5′ homology arm, a first polynucleotide encoding a first polypeptide of interest, and a 3′ homology arm, OR a set of first PCR fragments comprising a first 5′ homology arm, a first 3′ homology arm, and a first polynucleotide encoding a first polypeptide of interest,
wherein the 3′ homology arm is identical to the 3′ homology arm of one or more LPP, and wherein the 5′ homology arm is identical to the 5′ homology arm of one or more LPP, and
c) cultivating the host cell at a first temperature allowing one or more first guide RNA of the at least one guide RNA to form a complex with the nuclease at one or more protospacer of one or more LPP, and wherein the complex promotes the homology directed repair by creating a double-strand break at or near the one or more protospacer sequence of the one or more LPP, wherein the homology directed repair comprises insertion of the first donor polynucleotide or set of first PCR fragments at the double-strand break.
215. The method according to any one of the previous paragraphs, wherein the first polypeptide is heterologous to the host cell.
216. The method according to any one of the previous paragraphs, wherein the first polynucleotide is operably linked to one or more control sequences that direct the production of the first polypeptide.
217. The method according to any one of the previous paragraphs, wherein at least one of the one or more control sequences is heterologous to the first polynucleotide.
218. The method according to any one of the previous paragraphs, wherein the first guide RNA is complementary to one or more first protospacers, wherein during step c) the complex is formed at one or more first protospacer of the one or more LPP, creating a double-strand break at or near the one or more first protospacer.
219. The method according to any one of the previous paragraphs, wherein the first guide RNA is complementary to one or more second protospacers, wherein during step c) the complex is formed at one or more second protospacer of the one or more LPP, creating a double-strand break at or near the one or more second protospacer.
220. The method according to any one of the previous paragraphs, wherein the first guide RNA is complementary to one or more third protospacers, wherein during step c) the complex is formed at one or more third protospacer of the one or more LPP, creating a double-strand break at or near the one or more third protospacer.
221. The method according to any one of the previous paragraphs, wherein the first guide RNA is complementary to one or more fourth protospacers, wherein during step c) the complex is formed at one or more fourth protospacer of the one or more LPP, creating a double-strand break at or near the one or more fourth protospacer.
222. The method according to any one of the previous paragraphs, wherein the first guide RNA is complementary to one or more fifth protospacers, wherein during step c) the complex is formed at one or more fifth protospacer of the one or more LPP, creating a double-strand break at or near the one or more fifth protospacer.
223. The method according to any one of the previous paragraphs, wherein during step c) the first temperature allows the one or more first guide RNA to form a complex at one or more protospacers of one LPP, and wherein the complex promotes the homology directed repair by creating a double-strand break at or near the one or more protospacer sequence of the one LPP, wherein the homology directed repair comprises insertion of the first donor polynucleotide and/or set of first PCR fragments at the double-strand break to provide a host cell with one copy of the first donor polynucleotide in its genome.
224. The method according to any one of the previous paragraphs, wherein during step c) the first temperature allows the one or more first guide RNA to form a complex at one or more protospacers of two LPP, and wherein the complex promotes the homology directed repair by creating a double-strand break at or near the one or more protospacer sequence of the two LPP, wherein the homology directed repair comprises insertion of the first donor polynucleotide or set of first PCR fragments at the double-strand break to provide a host cell with two copies of the first donor polynucleotide in its genome.
225. The method according to any one of the previous paragraphs, wherein during step c) the first temperature allows the one or more first guide RNA to form a complex at one or more protospacers of three LPP, and wherein the complex promotes the homology directed repair by creating a double-strand break at or near the one or more protospacer sequence of the three LPP, wherein the homology directed repair comprises insertion of the first donor polynucleotide or set of first PCR fragments at the double-strand break to provide a host cell with three copies of the first donor polynucleotide in its genome.
226. The method according to any one of the previous paragraphs, wherein during step c) the first temperature allows the one or more first guide RNA to form a complex at one or more protospacers of four LPP, and wherein the complex promotes the homology directed repair by creating a double-strand break at or near the one or more protospacer sequence of the four LPP, wherein the homology directed repair comprises insertion of the first donor polynucleotide or set of first PCR fragments at the double-strand break to provide a host cell with four copies of the first donor polynucleotide in its genome.
227. The method according to any one of the previous paragraphs, wherein during step c) the first temperature allows the one or more first guide RNA to form a complex at one or more protospacers of five LPP, and wherein the complex promotes the homology directed repair by creating a double-strand break at or near the one or more protospacer sequence of the five LPP, wherein the homology directed repair comprises insertion of the first donor polynucleotide or set of first PCR fragments at the double-strand break to provide a host cell with five copies of the first donor polynucleotide in its genome.
228. The method according to any one of the previous paragraphs, wherein during step c) the first temperature allows the one or more first guide RNA to form a complex at one or more protospacers of six LPP, and wherein the complex promotes the homology directed repair by creating a double-strand break at or near the one or more protospacer sequence of the six LPP, wherein the homology directed repair comprises insertion of the first donor polynucleotide or set of first PCR fragments at the double-strand break to provide a host cell with six copies of the first donor polynucleotide in its genome.
229. The method according to any one of the previous paragraphs, wherein during step c) the first temperature allows the one or more first guide RNA to form a complex at one or more protospacers of 7 LPP, and wherein the complex promotes the homology directed repair by creating a double-strand break at or near the one or more protospacer sequence of the 7 LPP, wherein the homology directed repair comprises insertion of the first donor polynucleotide or set of first PCR fragments at the double-strand break to provide a host cell with 7 copies of the first donor polynucleotide in its genome.
230. The method according to any one of the previous paragraphs, wherein during step c) the first temperature allows the one or more first guide RNA to form a complex at one or more protospacers of 8 LPP, and wherein the complex promotes the homology directed repair by creating a double-strand break at or near the one or more protospacer sequence of the 8 LPP, wherein the homology directed repair comprises insertion of the first donor polynucleotide or set of first PCR fragments at the double-strand break to provide a host cell with 8 copies of the first donor polynucleotide in its genome.
231. The method according to any one of the previous paragraphs, wherein during step c) the first temperature allows the one or more first guide RNA to form a complex at one or more protospacers of 9 LPP, and wherein the complex promotes the homology directed repair by creating a double-strand break at or near the one or more protospacer sequence of the 9 LPP, wherein the homology directed repair comprises insertion of the first donor polynucleotide or set of first PCR fragments at the double-strand break to provide a host cell with 9 copies of the first donor polynucleotide in its genome.
232. The method according to any one of the previous paragraphs, wherein during step c) the first temperature allows the one or more first guide RNA to form a complex at one or more protospacers of 10 LPP, and wherein the complex promotes the homology directed repair by creating a double-strand break at or near the one or more protospacer sequence of the 10 LPP, wherein the homology directed repair comprises insertion of the first donor polynucleotide or set of first PCR fragments at the double-strand break to provide a host cell with 10 copies of the first donor polynucleotide in its genome.
233. The method according to any one of the previous paragraphs, wherein during step c) the first temperature allows the one or more first guide RNA to form a complex at one or more protospacers of 11 LPP, and wherein the complex promotes the homology directed repair by creating a double-strand break at or near the one or more protospacer sequence of the 11 LPP, wherein the homology directed repair comprises insertion of the first donor polynucleotide or set of first PCR fragments at the double-strand break to provide a host cell with 11 copies of the first donor polynucleotide in its genome.
234. The method according to any one of the previous paragraphs, wherein during step c) the first temperature allows the one or more first guide RNA to form a complex at one or more protospacers of 12 LPP, and wherein the complex promotes the homology directed repair by creating a double-strand break at or near the one or more protospacer sequence of the 12 LPP, wherein the homology directed repair comprises insertion of the first donor polynucleotide or set of first PCR fragments at the double-strand break to provide a host cell with 12 copies of the first donor polynucleotide in its genome.
235. The method according to any one of the previous paragraphs, wherein during step c) the first temperature allows the one or more first guide RNA to form a complex at one or more protospacers of 13 LPP, and wherein the complex promotes the homology directed repair by creating a double-strand break at or near the one or more protospacer sequence of the 13 LPP, wherein the homology directed repair comprises insertion of the first donor polynucleotide or set of first PCR fragments at the double-strand break to provide a host cell with 13 copies of the first donor polynucleotide in its genome.
236. The method according to any one of the previous paragraphs, wherein during step c) the first temperature allows the one or more first guide RNA to form a complex at one or more protospacers of 14 LPP, and wherein the complex promotes the homology directed repair by creating a double-strand break at or near the one or more protospacer sequence of the 14 LPP, wherein the homology directed repair comprises insertion of the first donor polynucleotide or set of first PCR fragments at the double-strand break to provide a host cell with 14 copies of the first donor polynucleotide in its genome.
237. The method according to any one of the previous paragraphs, wherein during step c) the first temperature allows the one or more first guide RNA to form a complex at one or more protospacers of 15 LPP, and wherein the complex promotes the homology directed repair by creating a double-strand break at or near the one or more protospacer sequence of the 15 LPP, wherein the homology directed repair comprises insertion of the first donor polynucleotide or set of first PCR fragments at the double-strand break to provide a host cell with 15 copies of the first donor polynucleotide in its genome.
238. The method according to any one of the previous paragraphs, wherein during step c) the first temperature allows the one or more first guide RNA to form a complex at one or more protospacers of 16 LPP, and wherein the complex promotes the homology directed repair by creating a double-strand break at or near the one or more protospacer sequence of the 16 LPP, wherein the homology directed repair comprises insertion of the first donor polynucleotide or set of first PCR fragments at the double-strand break to provide a host cell with 16 copies of the first donor polynucleotide in its genome.
239. The method according to any one of the previous paragraphs, wherein during step b) there are delivered at least two first guide RNAs, each first guide RNA forming a complex with the nuclease at a first, second, third, fourth, or fifth protospacer.
240. The method according to any one of the previous paragraphs, wherein the at least two first guide RNAs comprise at least two of: a 1^{st} first guide RNA, a 2^{nd} first guide RNA, a 3^{rd} first guide RNA, a 4^{th} first guide RNA, and a 5^{th} first guide RNA.
241. The method according to any one of the previous paragraphs, wherein the 1^{st} first guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 2^{nd} first guide RNA, to the polynucleotide sequence of 3^{rd} first guide RNA, to the polynucleotide sequence of the 4^{th} first guide RNA, and to the polynucleotide sequence of the 5^{th} first guide RNA.
242. The method according to any one of the previous paragraphs, wherein the 2^{nd} first guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} first guide RNA, to the polynucleotide sequence of 3^{rd} first guide RNA, to the polynucleotide sequence of the 4^{th} first guide RNA, and to the polynucleotide sequence of the 5^{th} first guide RNA.
243. The method according to any one of the previous paragraphs, wherein the 3^{rd} first guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} first guide RNA, to the polynucleotide sequence of 2^{nd} first guide RNA, to the polynucleotide sequence of the 4^{th} first guide RNA, and to the polynucleotide sequence of the 5^{th} first guide RNA.
244. The method according to any one of the previous paragraphs, wherein the 4^{th} first guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} first guide RNA, to the polynucleotide sequence of 2^{nd} first guide RNA, to the polynucleotide sequence of the 3^{rd} first guide RNA, and to the polynucleotide sequence of the 5^{th} first guide RNA.
245. The method according to any one of the previous paragraphs, wherein the 5^{th} first guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} first guide RNA, to the polynucleotide sequence of 2^{nd} first guide RNA, to the polynucleotide sequence of the 3^{rd} first guide RNA, and to the polynucleotide sequence of the 4^{th} first guide RNA.
246. The method according to any one of the previous paragraphs, wherein the one or more first guide RNA comprises a 1^{st} first guide RNA, having a sequence complementary to a first protospacer sequence, a second protospacer sequence, a third protospacer sequence, a fourth protospacer sequence, or a fifth protospacer sequence, preferably the 1^{st} first guide RNA is complementary to a first protospacer sequence.
247. The method according to any one of the previous paragraphs, wherein the one or more first guide RNA comprises a 2^{nd} first guide RNA, having a sequence complementary to a first protospacer sequence, a second protospacer sequence, a third protospacer sequence, a fourth protospacer sequence, or a fifth protospacer sequence, preferably the 2^{nd} first guide RNA is complementary to a second protospacer sequence.
248. The method according to any one of the previous paragraphs, wherein the one or more first guide RNA comprises a 3^{rd} first guide RNA, having a sequence complementary to a first protospacer sequence, a second protospacer sequence, a third protospacer sequence, a fourth protospacer sequence, or a fifth protospacer sequence, preferably the 3^{rd} first guide RNA is complementary to a third protospacer sequence.
249. The method according to any one of the previous paragraphs, wherein the one or more first guide RNA comprises a 4^{th} first guide RNA, having a sequence complementary to a first protospacer sequence, a second protospacer sequence, a third protospacer sequence, a fourth protospacer sequence, or a fifth protospacer sequence, preferably the 4^{th} first guide RNA is complementary to a fourth protospacer sequence.
250. The method according to any one of the previous paragraphs, wherein the one or more first guide RNA comprises a 5^{th} first guide RNA, having a sequence complementary to a first protospacer sequence, a second protospacer sequence, a third protospacer sequence, a fourth protospacer sequence, or a fifth protospacer sequence, preferably the 5^{th} first guide RNA is complementary to a fifth protospacer sequence.
251. The method according to any one of the previous paragraphs, wherein the at least one guide RNA comprises a first guide RNA and a second guide RNA, and wherein the method additionally comprises step d):
   d) cultivating the host cell at a second temperature allowing one or more second guide RNA of the at least one guide RNA to form a complex with the nuclease at one or more protospacer of the one or more LPP, and wherein the complex promotes the homology directed repair by creating a double-strand break at or near the one or more protospacer sequence of the one or more LPP, wherein the homology directed repair comprises insertion of the first donor polynucleotide or set of first PCR fragments at the double-strand break.
251a. The method according to any one of the previous paragraphs, wherein during step b) there is additionally delivered one or more second polynucleotide comprising in 5′ to 3′ direction: a 5′ homology arm, a second polynucleotide encoding a second polypeptide of interest, and a 3′ homology arm, OR a set of one or more second PCR fragments comprising a second 3′ homology arm, a second 5′ homology arm, and a second polynucleotide encoding a second polypeptide of interest, wherein the 3′ homology arm is identical to the 3′ homology arm of one or more LPP and wherein the 5′ homology arm is identical to the 5′ homology arm of one or more LPP, and wherein during step c) the homology directed repair comprises insertion of the second donor polynucleotide or set of second PCR fragments at the double-strand break.
251b. The method according to any one of the previous paragraphs, wherein during step b) there is additionally delivered one or more first polynucleotide comprising in 5′ to 3′ direction: a second 5′ homology arm, a first polynucleotide encoding the first polypeptide of interest, and a second 3′ homology arm, OR a set of one or more first PCR fragments comprising a second 3′ homology arm, a second 5′ homology arm, and the first polynucleotide encoding the first polypeptide of interest, wherein the second 3′ homology arms and the second 5′ homology arms have less than 100% sequence identity to the 3′ and 5′ homology arms of the at least two LPP, respectively, e.g., less than 99%, less than 98%, less than 97%, less than 96%, less than 95%, less than 90%, less thank 85%, less than 80%, less than 75%, less than 70%, or less than 65%, and wherein during step c) the homology directed repair comprises insertion of the first donor polynucleotide or set of PCR fragments at the double-strand break between a second 3′ homology arm and a second 5′ homology arm.
252. The method according to any one of the previous paragraphs, wherein the second guide RNA is complementary to one or more first protospacers, wherein during step d) the complex is formed at one or more first protospacer of the one or more LPP, creating a double-strand break at or near the one or more first protospacer.
253. The method according to any one of the previous paragraphs, wherein the second guide RNA is complementary to one or more second protospacers, wherein during step d) the complex is formed at one or more second protospacer of the one or more LPP, creating a double-strand break at or near the one or more second protospacer.
254. The method according to any one of the previous paragraphs, wherein the second guide RNA is complementary to one or more third protospacers, wherein during step d) the complex is formed at one or more third protospacer of the one or more LPP, creating a double-strand break at or near the one or more third protospacer.
255. The method according to any one of the previous paragraphs, wherein the second guide RNA is complementary to one or more fourth protospacers, wherein during step d) the complex is formed at one or more fourth protospacer of the one or more LPP, creating a double-strand break at or near the one or more fourth protospacer.
256. The method according to any one of the previous paragraphs, wherein the second guide RNA is complementary to one or more fifth protospacers, wherein during step d) the complex is formed at one or more fifth protospacer of the one or more LPP, creating a double-strand break at or near the one or more fifth protospacer.
257. The method according to any one of the previous paragraphs, wherein during step d) the second temperature allows the one or more second guide RNA to form a complex at one or more protospacers of one LPP, and wherein the complex promotes the homology directed repair by creating a double-strand break at or near the one or more protospacer sequence of the one LPP, wherein the homology directed repair comprises insertion of the first donor polynucleotide and/or set of first PCR fragments at the double-strand break to provide a host cell with at least one copy of the first donor polynucleotide in its genome.
258. The method according to any one of the previous paragraphs, wherein during step d) the second temperature allows the one or more second guide RNA to form a complex at one or more protospacers of two LPP, and wherein the complex promotes the homology directed repair by creating a double-strand break at or near the one or more protospacer sequence of the two LPP, wherein the homology directed repair comprises insertion of the first donor polynucleotide or set of first PCR fragments at the double-strand break to provide a host cell with at least two copies of the first donor polynucleotide in its genome.
259. The method according to any one of the previous paragraphs, wherein during step d) the second temperature allows the one or more second guide RNA to form a complex at one or more protospacers of three LPP, and wherein the complex promotes the homology directed repair by creating a double-strand break at or near the one or more protospacer sequence of the three LPP, wherein the homology directed repair comprises insertion of the first donor polynucleotide or set of first PCR fragments at the double-strand break to provide a host cell with at least three copies of the first donor polynucleotide in its genome.
260. The method according to any one of the previous paragraphs, wherein during step d) the second temperature allows the one or more second guide RNA to form a complex at one or more protospacers of four LPP, and wherein the complex promotes the homology directed repair by creating a double-strand break at or near the one or more protospacer sequence of the four LPP, wherein the homology directed repair comprises insertion of the first donor polynucleotide or set of first PCR fragments at the double-strand break to provide a host cell with at least four copies of the first donor polynucleotide in its genome.
261. The method according to any one of the previous paragraphs, wherein during step d) the second temperature allows the one or more second guide RNA to form a complex at one or more protospacers of five LPP, and wherein the complex promotes the homology directed repair by creating a double-strand break at or near the one or more protospacer sequence of the five LPP, wherein the homology directed repair comprises insertion of the first donor polynucleotide or set of first PCR fragments at the double-strand break to provide a host cell with at least five copies of the first donor polynucleotide in its genome.
262. The method according to any one of the previous paragraphs, wherein during step d) the second temperature allows the one or more second guide RNA to form a complex at one or more protospacers of six LPP, and wherein the complex promotes the homology directed repair by creating a double-strand break at or near the one or more protospacer sequence of the six LPP, wherein the homology directed repair comprises insertion of the first donor polynucleotide or set of first PCR fragments at the double-strand break to provide a host cell with at least six copies of the first donor polynucleotide in its genome.
263. The method according to any one of the previous paragraphs, wherein during step d) the second temperature allows the one or more second guide RNA to form a complex at one or more protospacers of 8 LPP, and wherein the complex promotes the homology directed repair by creating a double-strand break at or near the one or more protospacer sequence of the 8 LPP, wherein the homology directed repair comprises insertion of the first donor polynucleotide or set of first PCR fragments at the double-strand break to provide a host cell with at least 8 copies of the first donor polynucleotide in its genome.
264. The method according to any one of the previous paragraphs, wherein during step d) the second temperature allows the one or more second guide RNA to form a complex at one or more protospacers of 16 LPP, and wherein the complex promotes the homology directed repair by creating a double-strand break at or near the one or more protospacer sequence of the 16 LPP, wherein the homology directed repair comprises insertion of the first donor polynucleotide or set of first PCR fragments at the double-strand break to provide a host cell with at least 16 copies of the first donor polynucleotide in its genome.
265. The method according to any one of the previous paragraphs, wherein during step b) there are delivered at least two second guide RNAs, each second guide RNA forming a complex with the nuclease at a first, second, third, fourth, or fifth protospacer.
266. The method according to any one of the previous paragraphs, wherein the at least two second guide RNAs comprise at least two of: a 1^{st} second guide RNA, a 2^{nd} second guide RNA, a 3^{rd} second guide RNA, a 4^{th} second guide RNA, and a 5^{th} second guide RNA.
267. The method according to any one of the previous paragraphs, wherein the 1^{st} second guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 2^{nd} second guide RNA, to the polynucleotide sequence of 3^{rd} second guide RNA, to the polynucleotide sequence of the 4^{th} second guide RNA, and to the polynucleotide sequence of the 5^{th} second guide RNA.
268. The method according to any one of the previous paragraphs, wherein the 2^{nd} second guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} second guide RNA, to the polynucleotide sequence of 3^{rd} second guide RNA, to the polynucleotide sequence of the 4^{th} second guide RNA, and to the polynucleotide sequence of the 5^{th} second guide RNA.
269. The method according to any one of the previous paragraphs, wherein the 3^{rd} second guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} second guide RNA, to the polynucleotide sequence of 2^{nd} second guide RNA, to the polynucleotide sequence of the 4^{th} second guide RNA, and to the polynucleotide sequence of the 5^{th} second guide RNA.
270. The method according to any one of the previous paragraphs, wherein the 4^{th} second guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} second guide RNA, to the polynucleotide sequence of 2^{nd} second guide RNA, to the polynucleotide sequence of the 3^{rd} second guide RNA, and to the polynucleotide sequence of the 5^{th} second guide RNA.
271. The method according to any one of the previous paragraphs, wherein the 5^{th} second guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} second guide RNA, to the polynucleotide sequence of 2^{nd} second guide RNA, to the polynucleotide sequence of the 3^{rd} second guide RNA, and to the polynucleotide sequence of the 4^{th} second guide RNA.
272. The method according to any one of the previous paragraphs, wherein the one or more second guide RNA comprises a 1^{st} second guide RNA, having a sequence complementary to a first protospacer sequence, a second protospacer sequence, a third protospacer sequence, a fourth protospacer sequence, or a fifth protospacer sequence, preferably the 1^{st} second guide RNA is complementary to a first protospacer sequence.
273. The method according to any one of the previous paragraphs, wherein the one or more second guide RNA comprises a 2^{nd} second guide RNA, having a sequence complementary to a first protospacer sequence, a second protospacer sequence, a third protospacer sequence, a fourth protospacer sequence, or a fifth protospacer sequence, preferably the 2^{nd} second guide RNA is complementary to a second protospacer sequence.
274. The method according to any one of the previous paragraphs, wherein the one or more second guide RNA comprises a 3^{rd} second guide RNA, having a sequence complementary to a first protospacer sequence, a second protospacer sequence, a third protospacer sequence, a fourth protospacer sequence, or a fifth protospacer sequence, preferably the 3^{rd} second guide RNA is complementary to a third protospacer sequence.
275. The method according to any one of the previous paragraphs, wherein the one or more second guide RNA comprises a 4^{th} second guide RNA, having a sequence complementary to a first protospacer sequence, a second protospacer sequence, a third protospacer sequence, a fourth protospacer sequence, or a fifth protospacer sequence, preferably the 4^{th} second guide RNA is complementary to a fourth protospacer sequence.
276. The method according to any one of the previous paragraphs, wherein the one or more second guide RNA comprises a 5^{th} second guide RNA, having a sequence complementary to a first protospacer sequence, a second protospacer sequence, a third protospacer sequence, a fourth protospacer sequence, or a fifth protospacer sequence, preferably the 5^{th} second guide RNA is complementary to a fifth protospacer sequence.
277. The method according to any one of the previous paragraphs, wherein the at least one guide RNA comprises a first guide RNA, a second guide RNA, and a third guide RNA, and wherein the method additionally comprises the step
e) cultivating the host cell at a third temperature allowing one or more third guide RNA of the at least one guide RNA to form a complex with the nuclease at one or more protospacer of the one or more LPP, and wherein the complex promotes the homology directed repair by creating a double-strand break at or near the one or more protospacer sequence of the one or more LPP, wherein the homology directed repair comprises insertion of the first donor polynucleotide and/or set of first PCR fragments at the double-strand break.
278. The method according to any one of the previous paragraphs, wherein the third guide RNA is complementary to one or more first protospacers, wherein during step e) the complex is formed at one or more first protospacer of the one or more LPP, creating a double-strand break at or near the one or more first protospacer.
279. The method according to any one of the previous paragraphs, wherein the third guide RNA is complementary to one or more second protospacers, wherein during step e) the complex is formed at one or more second protospacer of the one or more LPP, creating a double-strand break at or near the one or more second protospacer.
280. The method according to any one of the previous paragraphs, wherein the third guide RNA is complementary to one or more third protospacers, wherein during step e) the complex is formed at one or more third protospacer of the one or more LPP, creating a double-strand break at or near the one or more third protospacer.
281. The method according to any one of the previous paragraphs, wherein the third guide RNA is complementary to one or more fourth protospacers, wherein during step e) the complex is formed at one or more fourth protospacer of the one or more LPP, creating a double-strand break at or near the one or more fourth protospacer.
282. The method according to any one of the previous paragraphs, wherein the third guide RNA is complementary to one or more fifth protospacers, wherein during step e) the complex is formed at one or more fifth protospacer of the one or more LPP, creating a double-strand break at or near the one or more fifth protospacer.
283. The method according to any one of the previous paragraphs, wherein during step e) the third temperature allows the one or more third guide RNA to form a complex at one or more protospacers of one LPP, and wherein the complex promotes the homology directed repair by creating a double-strand break at or near the one or more protospacer sequence of the one LPP, wherein the homology directed repair comprises insertion of the first donor polynucleotide or set of first PCR fragments at the double-strand break to provide a host cell with at least one copy of the first donor polynucleotide in its genome.
284. The method according to any one of the previous paragraphs, wherein during step e) the third temperature allows the one or more third guide RNA to form a complex at one or more protospacers of two LPP, and wherein the complex promotes the homology directed repair by creating a double-strand break at or near the one or more protospacer sequence of the two LPP, wherein the homology directed repair comprises insertion of the first donor polynucleotide or set of first PCR fragments at the double-strand break to provide a host cell with at least two copies of the first donor polynucleotide in its genome.
285. The method according to any one of the previous paragraphs, wherein during step e) the third temperature allows the one or more third guide RNA to form a complex at one or more protospacers of three LPP, and wherein the complex promotes the homology directed repair by creating a double-strand break at or near the one or more protospacer sequence of the three LPP, wherein the homology directed repair comprises insertion of the first donor polynucleotide or set of first PCR fragments at the double-strand break to provide a host cell with at least three copies of the first donor polynucleotide in its genome.
286. The method according to any one of the previous paragraphs, wherein during step e) the third temperature allows the one or more third guide RNA to form a complex at one or more protospacers of four LPP, and wherein the complex promotes the homology directed repair by creating a double-strand break at or near the one or more protospacer sequence of the four LPP, wherein the homology directed repair comprises insertion of the first donor polynucleotide or set of first PCR fragments at the double-strand break to provide a host cell with at least four copies of the first donor polynucleotide in its genome.
287. The method according to any one of the previous paragraphs, wherein during step e) the third temperature allows the one or more third guide RNA to form a complex at one or more protospacers of five LPP, and wherein the complex promotes the homology directed repair by creating a double-strand break at or near the one or more protospacer sequence of the five LPP, wherein the homology directed repair comprises insertion of the first donor polynucleotide or set of first PCR fragments at the double-strand break to provide a host cell with at least five copies of the first donor polynucleotide in its genome.
288. The method according to any one of the previous paragraphs, wherein during step e) the third temperature allows the one or more third guide RNA to form a complex at one or more protospacers of six LPP, and wherein the complex promotes the homology directed repair by creating a double-strand break at or near the one or more protospacer sequence of the six LPP, wherein the homology directed repair comprises insertion of the first donor polynucleotide or set of first PCR fragments at the double-strand break to provide a host cell with at least six copies of the first donor polynucleotide in its genome.
289. The method according to any one of the previous paragraphs, wherein during step e) the third temperature allows the one or more third guide RNA to form a complex at one or more protospacers of 8 LPP, and wherein the complex promotes the homology directed repair by creating a double-strand break at or near the one or more protospacer sequence of the 8 LPP, wherein the homology directed repair comprises insertion of the first donor polynucleotide or set of first PCR fragments at the double-strand break to provide a host cell with at least 8 copies of the first donor polynucleotide in its genome.
290. The method according to any one of the previous paragraphs, wherein during step e) the third temperature allows the one or more third guide RNA to form a complex at one or more protospacers of 16 LPP, and wherein the complex promotes the homology directed repair by creating a double-strand break at or near the one or more protospacer sequence of the 16 LPP, wherein the homology directed repair comprises insertion of the first donor polynucleotide or set of first PCR fragments at the double-strand break to provide a host cell with at least 16 copies of the first donor polynucleotide in its genome.
291. The method according to any one of the previous paragraphs, wherein during step b) there are delivered at least two third guide RNAs, each third guide RNA forming a complex with the nuclease at a first, second, third, fourth, or fifth protospacer.
292. The method according to any one of the previous paragraphs, wherein the at least two third guide RNAs comprise at least two of: a 1^{st} third guide RNA, a 2^{nd} third guide RNA, a 3^{rd} third guide RNA, a 4th third guide RNA, and a 5^{th} third guide RNA.
293. The method according to any one of the previous paragraphs, wherein the 1^{st} third guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 2^{nd} third guide RNA, to the polynucleotide sequence of 3^{rd} third guide RNA, to the polynucleotide sequence of the 4^{th} third guide RNA, and to the polynucleotide sequence of the 5^{th} third guide RNA.
294. The method according to any one of the previous paragraphs, wherein the 2^{nd} third guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} third guide RNA, to the polynucleotide sequence of 3^{rd} third guide RNA, to the polynucleotide sequence of the 4^{th} third guide RNA, and to the polynucleotide sequence of the 5^{th} third guide RNA.
295. The method according to any one of the previous paragraphs, wherein the 3^{rd} third guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} third guide RNA, to the polynucleotide sequence of 2^{nd} third guide RNA, to the polynucleotide sequence of the 4^{th} third guide RNA, and to the polynucleotide sequence of the 5^{th} third guide RNA.
296. The method according to any one of the previous paragraphs, wherein the 4^{th} third guide RNA has a polynucleotide sequence with a sequence identity of below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} third guide RNA, to the polynucleotide sequence of 2^{nd} third guide RNA, to the polynucleotide sequence of the 3^{rd} third guide RNA, and to the polynucleotide sequence of the 5^{th} third guide RNA.
297. The method according to any one of the previous paragraphs, wherein the 5^{th} third guide RNA has a polynucleotide sequence with a sequence identity below 100%, such as below 99%, below 98%, below 97%, below 96%, below 95%, below 90%, below 85%, below 80%, below 75%, below 70%, below 65%, below 60%, below 55%, below 50%, below 45%, below 40%, below 35%, below 30%, below 25%, below 20%, below 15%, or below 10% to the polynucleotide sequence of the 1^{st} third guide RNA, to the polynucleotide sequence of 2^{nd} third guide RNA, to the polynucleotide sequence of the 3^{rd} third guide RNA, and to the polynucleotide sequence of the 4^{th} third guide RNA.
298. The method according to any one of the previous paragraphs, wherein the one or more third guide RNA comprises a 1^{st} third guide RNA, having a sequence complementary to a first protospacer sequence, a second protospacer sequence, a third protospacer sequence, a fourth protospacer sequence, or a fifth protospacer sequence, preferably the 1^{st} third guide RNA is complementary to a first protospacer sequence.
299. The method according to any one of the previous paragraphs, wherein the one or more third guide RNA comprises a 2^{nd} third guide RNA, having a sequence complementary to a first protospacer sequence, a second protospacer sequence, a third protospacer sequence, a fourth protospacer sequence, or a fifth protospacer sequence, preferably the 2^{nd} third guide RNA is complementary to a second protospacer sequence.
300. The method according to any one of the previous paragraphs, wherein the one or more third guide RNA comprises a 3^{rd} third guide RNA, having a sequence complementary to a first protospacer sequence, a second protospacer sequence, a third protospacer sequence, a fourth protospacer sequence, or a fifth protospacer sequence, preferably the 3^{rd} third guide RNA is complementary to a third protospacer sequence.
301. The method according to any one of the previous paragraphs, wherein the one or more third guide RNA comprises a 4^{th} third guide RNA, having a sequence complementary to a first protospacer sequence, a second protospacer sequence, a third protospacer sequence, a fourth protospacer sequence, or a fifth protospacer sequence, preferably the 4^{th} third guide RNA is complementary to a fourth protospacer sequence.
302. The method according to any one of the previous paragraphs, wherein the one or more third guide RNA comprises a 5^{th} third guide RNA, having a sequence complementary to a first protospacer sequence, a second protospacer sequence, a third protospacer sequence, a fourth protospacer sequence, or a fifth protospacer sequence, preferably the 5^{th} third guide RNA is complementary to a fifth protospacer sequence.
303. The method according to any one of the previous paragraphs, wherein the at least one guide RNA comprises a first guide RNA, a second guide RNA, a third guide RNA, and a fourth guide RNA, and wherein the method additionally comprises the step
f) cultivating the host cell at a fourth temperature allowing one or more fourth guide RNA of the at least one guide RNA to form a complex with the nuclease at one or more protospacer of the one or more LPP, and wherein the complex promotes the homology directed repair by creating a double-strand break at or near the one or more protospacer sequence of the one or more LPP, wherein the homology directed repair comprises insertion of the first donor polynucleotide or set of first PCR fragments at the double-strand break.
304. The method according to any one of the previous paragraphs, wherein the at least one guide RNA comprises a first guide RNA, a second guide RNA, a third guide RNA, a fourth guide RNA, and a fifth guide RNA, and wherein the method additionally comprises the step
g) cultivating the host cell at a fifth temperature allowing one or more fifth guide RNA of the at least one guide RNA to form a complex with the nuclease at one or more protospacer of the one or more LPP, and wherein the complex promotes the homology directed repair by creating a double-strand break at or near the one or more protospacer sequence of the one or more LPP, wherein the homology directed repair comprises insertion of the first donor polynucleotide or set of first PCR fragments at the double-strand break.
305. The method according to any one of the previous paragraphs, wherein the first temperature is in the range of 15-50 C°, such as in the range of 22-42 C°, such as in the range of 23-39 C°, such as in the range of 25-37 C°.
306. The method according to any one of the previous paragraphs, wherein the second temperature is in the range of 15-50 C°, such as in the range of 22-42 C°, such as in the range of 23-39 C°, such as in the range of 25-37 C°.
307. The method according to any one of the previous paragraphs, wherein the third temperature is in the range of 15-50 C°, such as in the range of 22-42 C°, such as in the range of 23-39 C°, such as in the range of 25-37 C°.
308. The method according to any one of the previous paragraphs, wherein the fourth temperature is in the range of 15-50 C°, such as in the range of 22-42 C°, such as in the range of 23-39 C°, such as in the range of 25-37 C°.
309. The method according to any one of the previous paragraphs, wherein the fifth temperature is in the range of 15-50 C°, such as in the range of 22-42 C°, such as in the range of 23-39 C°, such as in the range of 25-37 C°.
310. The method according to any one of the previous paragraphs, wherein the first temperature and the second temperature are in the range of 15-50 C°, such as in the range of 22-42 C°, such as in the range of 23-39 C°, such as in the range of 25-37 C°.
311. The method according to any one of the previous paragraphs, wherein the first temperature, the second temperature, and the third temperature are in the range of 15-50 C°, such as in the range of 22-42 C°, such as in the range of 23-39 C°, such as in the range of 25-37 C°.
312. The method according to any one of the previous paragraphs, wherein the first temperature, the second temperature, the third temperature, and the fourth temperature are in the range of 15-50 C°, such as in the range of 22-42 C°, such as in the range of 23-39 C°, such as in the range of 25-37 C°.
313. The method according to any one of the previous paragraphs, wherein the first temperature, the second temperature, the third temperature, the fourth temperature, and the fifth temperature are in the range of 15-50 C°, such as in the range of 22-42 C°, such as in the range of 23-39 C°, such as in the range of 25-37 C°.
314. The method according to any one of the previous paragraphs, wherein the first temperature differs from the second temperature by at least 0.5 C°, at least 1 C°, at least 1.5 C°, at least 2 C°, at least 2.5 C°, at least 3 C°, at least 3.5 C°, at least 4 C°, at least 4.5 C°, at least 5 C°, at least 5.5 C°, or at least 6 C°.
315. The method according to any one of the previous paragraphs, wherein the second temperature differs from the third temperature by at least 0.5 C°, at least 1 C°, at least 1.5 C°, at least 2 C°, at least 2.5 C°, at least 3 C°, at least 3.5 C°, at least 4 C°, at least 4.5 C°, at least 5 C°, at least 5.5 C°, or at least 6 C°
316. The method according to any one of the previous paragraphs, wherein the third temperature differs from the fourth temperature by at least 0.5 C°, at least 1 C°, at least 1.5 C°, at least 2 C°, at least 2.5 C°, at least 3 C°, at least 3.5 C°, at least 4 C°, at least 4.5 C°, at least 5 C°, at least 5.5 C°, or at least 6 C°
317. The method according to any one of the previous paragraphs, wherein the fourth temperature differs from the fifth temperature by at least 0.5 C°, at least 1 C°, at least 1.5 C°, at least 2 C°, at least 2.5 C°, at least 3 C°, at least 3.5 C°, at least 4 C°, at least 4.5 C°, at least 5 C°, at least 5.5 C°, or at least 6 C°
318. The method according to any one of the previous paragraphs, wherein the first temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
319. The method according to any one of the previous paragraphs, wherein the second temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
320. The method according to any one of the previous paragraphs, wherein the third temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
321. The method according to any one of the previous paragraphs, wherein the fourth temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
322. The method according to any one of the previous paragraphs, wherein the fifth temperature is selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
323. The method according to any one of the previous paragraphs, wherein the first temperature and the second temperature are selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
324. The method according to any one of the previous paragraphs, wherein the first temperature, the second temperature, and the third temperature are selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
325. The method according to any one of the previous paragraphs, wherein the first temperature, the second temperature, the third temperature, and the fourth temperature are selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
326. The method according to any one of the previous paragraphs, wherein the first temperature, the second temperature, the third temperature, the fourth temperature, and the fifth temperature are selected from the list of 22 C°, 23 C°, 24 C°, 25 C°, 26 C°, 27 C°, 28 C°, 29 C°, 30 C°, 31 C°, 32 C°, 33 C°, 34 C°, 35 C°, 36 C°, 37 C°, 38 C°, 39 C°, 40 C°, 41 C°, 42 C°, 43 C°, 44 C°, 45 C°, 46 C°, 47 C°, 48 C°, 49 C°, and 50 C°.
327. The method according to any one of the previous paragraphs, wherein the first temperature is in the range of 22-40 C°, and wherein the second temperature is in the range of 35-50 C°.
328. The method according to any one of the previous paragraphs, wherein the first donor polynucleotide does not comprise a selectable marker or counter-selectable marker.
329. The method according to any one of the previous paragraphs, wherein the first donor polynucleotide comprises a selectable or counter-selectable marker.
330. The method according to any one of the previous paragraphs, wherein during step b) there is furthermore delivered a second donor polynucleotide comprising in 5′ to 3′ direction: a 5′ homology arm, a second polynucleotide encoding a second polypeptide of interest, and a 3′ homology arm, wherein the 3′ homology arm is identical to the 3′ homology arm of the host cell, and wherein the 5′ homology arm is identical to the 5′ homology arm of the host cell.
331. The method according to any one of the previous paragraphs, wherein the first polypeptide of interest comprises a brazzein, a casein, a patatin, an ovalbumin, an osteopontin, an ovotransferrin, an ovomucin, an ovomucoid, an ovostatin, a glycomacropeptide, a lactoferrin, an alpha-lactalbumin, e.g., bovine alpha-lactalbumin, a beta-lactalbumin and/or a collagen.
332. The method according to any one of the previous paragraphs, wherein the first polypeptide of interest comprises a therapeutic polypeptide selected from the group consisting of an antibody, an antibody fragment, an antibody-based drug, a Fc fusion protein, an anticoagulant, a blood factor, a bone morphogenetic protein, an engineered protein scaffold, an enzyme, a growth factor, a blood clotting factor, a hormone, an interferon (such as an interferon alpha-2b), an interleukin, a lactoferrin, an alpha-lactalbumin, a beta-lactalbumin, an ovomucoid, an ovostatin, a cytokine, an obestatin, a human galactosidase (such as an human alpha-galactosidase A), a vaccine, a protein vaccine, and a thrombolytic.
333. The method according to any one of the previous paragraphs, wherein the first polypeptide of interest comprises an enzyme.
334. The method according to any one of the previous paragraphs, wherein the enzyme is selected from the list of a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.
335. The method according to any one of the previous paragraphs, wherein the second polypeptide of interest comprises a polypeptide selected from the list of a transcription factor (e.g. amyR), a chaperone (e.g. BipA, PdiA, or PdiB), a heme pathway enzyme (e.g. encoded by hemA, or hemB), or a glycosylation pathway enzyme (e.g. a Mannosidase I, a GlcNAc transferase, a galactosyltransferase, a sialyltransferase, an oligosaccharyltransferase, or an glucosidase).
336. The method according to any one of the previous paragraphs, wherein the method additionally comprises step h), subsequent of any of steps c), d), e), f) or g):
   providing a host cell comprising a first polynucleotide in one or more LPP, said host cell being generated with the method of any of the previous paragraphs, and said host cell comprising at least one LPP not comprising the first polynucleotide (=empty LPP),
   delivering to the host cell a RNA-guided DNA nuclease, and at least one guide RNA directed to one or more protospacer of one or more empty LPP, and
   cultivating the host cell at a temperature allowing the at least one guide RNA to form a complex with the nuclease at the one or more empty LPP, wherein the complex promotes the homology directed repair by creating a double-strand break at or near the one or more protospacer sequence of the one or more empty LPP, wherein the homology directed repair comprises insertion of the first polynucleotide at the double-strand break of the empty LPP.
337. A method of producing a polypeptide of interest, comprising cultivating a multi-copy host cell, said cell being generated by the method of any preceding paragraphs, under conditions conducive for production of the polypeptide.
338. The method of paragraph 337, further comprising recovering the polypeptide of interest.

1. A host cell comprising in its genome at least two landing pad polynucleotide (LPP) sequences,
each LPP sequence comprising in 5′ to 3′ direction:
a 5′ homology arm,
two or more protospacer sequences, and
a 3′ homology arm,
wherein the polynucleotide sequence of the 5′ homology arm is identical for at least two LPP sequences, and
wherein the polynucleotide sequence of the 3′ homology arm is identical for at least two LPP sequences.

2. The host cell according to claim 1, wherein at least one protospacer of the at least two protospacers is present in at least two LPP.

3. The host cell according to claim 1, wherein the polynucleotide sequence of the 5′ homology arm is identical for all of the at least two LPP sequences.

4. The host cell according to claim 1, wherein the polynucleotide sequence of the 3′ homology arm is identical for all of the at least two LPP sequences.

5. The host cell according to claim 1, wherein the host cell comprises at least three LPP sequences.

6. The host cell according to claim 1, wherein at least one LPP is located on the coding strand (3′ to 5′ direction), and wherein at least one LPP is located on the template strand (5′ to 3′ direction).

7. The host cell according to claim 1, wherein the host cell is deficient of a non-homologous end joining (NHEJ) mechanism.

8. The host cell according to claim 1, wherein each 3′ homology arm and each 5′ homology arm has a length of at least 10 nt.

9. The host cell according to claim 1, wherein each of the at least two LPP comprises a first protospacer sequence with a polynucleotide sequence identical for each first protospacer of each at least two LPP.

10. The host cell according to claim 1, wherein each of the at least two LPP comprises a second protospacer sequence with a polynucleotide sequence which is unique for each second protospacer of each at least two LPP.

11. The host cell according to claim 1, wherein the host cell is a fungal cell.

12. The host cell according to claim 1, wherein the host cell is a yeast recombinant host cell.

13. The host cell according to any claim 1, wherein the host cell is a filamentous fungal recombinant host cell.

14. The host cell according to claim 1, wherein the host cell is a prokaryotic recombinant host cell.

15. The host cell according to claim 1, wherein the host cell is a mammalian cell.

16. A method for generating a multi-copy host cell, comprising the steps of
a) providing a host cell according to claim 1,
b) delivering to the host cell a gene editing system comprising:
a RNA-guided DNA nuclease,
at least one guide RNA directed to one or more protospacer of the one or more LPP sequences,
a first donor polynucleotide comprising in 5′ to 3′ direction: a 5′ homology arm, a first polynucleotide encoding a first polypeptide of interest, and a 3′ homology arm, OR
a set of first PCR fragments comprising a first 5′ homology arm, a first 3′ homology arm, and a first polynucleotide encoding a first polypeptide of interest,
wherein the 3′ homology arm and/or first 3′ homology arm is identical to the 3′ homology arm of one or more LPP and wherein the 5′ homology arm and/or first 5′ homology arm is identical to the 5′ homology arm of one or more LPP, and
c) cultivating the host cell at a first temperature allowing one or more first guide RNA of the at least one guide RNA to form a complex with the nuclease at one or more protospacer of one or more LPP, and wherein the complex promotes the homology directed repair by creating a double-strand break at or near the one or more protospacer sequence of the one or more LPP, wherein the homology directed repair comprises insertion of the first donor polynucleotide and/or set of first PCR fragments at the double-strand break.

17. The method according to claim 16, wherein the at least one guide RNA comprises a first guide RNA and a second guide RNA, and wherein the method additionally comprises step d):
d) cultivating the host cell at a second temperature allowing one or more second guide RNA of the at least one guide RNA to form a complex with the nuclease at one or more protospacer of the one or more LPP, and wherein the complex promotes the homology directed repair by creating a double-strand break at or near the one or more protospacer sequence of the one or more LPP, wherein the homology directed repair comprises insertion of the first donor polynucleotide at the double-strand break.

18. The method according to claim 16, wherein the first temperature differs from the second temperature by at least 0.5 C°.

19. The method according to claim 16, wherein the method, subsequent of any of steps c) or d), additionally comprises step h):
providing a host cell comprising a first polynucleotide in one or more LPP, said host cell being generated with the method of any of claims 16 to 18, and said host cell comprising at least one LPP not comprising the first polynucleotide (=empty LPP),
delivering to the host cell a RNA-guided DNA nuclease, and at least one guide RNA directed to one or more protospacer of one or more empty LPP, and
cultivating the host cell at a temperature allowing the at least one guide RNA to form a complex with the nuclease at the one or more empty LPP, wherein the complex promotes the homology directed repair by creating a double-strand break at or near the one or more protospacer sequence of the one or more empty LPP, wherein the homology directed repair comprises insertion of the first polynucleotide at the double-strand break of the empty LPP.

20. A method of producing a polypeptide of interest, comprising cultivating a multi-copy host cell under conditions conducive for production of the polypeptide, which multi-copy host cell is generated by the method according to claim 16.
